(19) United States
(12) Patent Application Publication
(10) Pub. No.: US 2016/0252516 A1
(43) Pub. Date: Sep. 1, 2016

(54) SYSTEM AND METHOD FOR HIGH THROUGHPUT MASS SPECTROMETRIC ANALYSIS OF PROTEOME SAMPLES

(71) Applicant: NORTHWESTERN UNIVERSITY, Evanston, IL (US)

(72) Inventors: Ki Hun KIM, Evanston, IL (US); John C. TRAN, Halifax (CA); Philip D. Compton, Evanston, IL (US); Neil L. Kelleher, Evanston, IL (US); Phillip Y. CHU, Newark, DE (US); Owen SKINNER, Chicago, IL (US)

(21) Appl. No.: 15/026,655

(22) PCT Filed: Oct. 3, 2014
(86) PCT No.: PCT/US14/59199

§ 371 Date: Apr. 1, 2016

Related U.S. Application Data
(60) Provisional application No. 61/886,428, filed on Oct. 3, 2013.

(51) Int. Cl. G01N 33/68; H01J 49/02; H01J 49/00; B01D 57/02

(52) U.S. Cl. CPC G01N 33/6842; G01N 33/6848; B01D 57/02; H01J 49/02; H01J 49/0031; G01N 2570/00; G01N 2560/00

(57) ABSTRACT

Disclosed herein is a system and method for analyzing a specimen containing the proteome by mass spectrometry. The system includes a protein separation module; a matrix processing module; and a mass spectrometer module. The protein separation module, the matrix processing module and the mass spectrometer are in fluid communication with one another.

### CROSS-REFERENCE TO RELATED APPLICATIONS

This application claims benefit of priority under 35 U.S.C. 119 to U.S. provisional patent application Ser. No. 61/886,428, filed Oct. 3, 2013, and entitled “SYSTEM AND METHOD FOR HIGH THROUGHPUT MASS SPECTROMETRIC ANALYSIS OF PROTEOME SAMPLES,” the content of which is herein incorporated by reference in its entirety.

### BACKGROUND

1. Technical Field

The present disclosure relates to on-line systems and methods for high throughput mass spectrometric analysis of proteome samples. In particular, on-line systems and sub-systems are disclosed that use a combination of capillary gel electrophoresis, modified AF4-mediated sample processing and mass spectrometric analysis of protein-containing clinical samples.

2. Description of Related Art

The proteome represents the totality of expressed proteins from the genome. To a significant extent, the structural characterization of proteins relies on determining the primary structure (amino acid sequence and covalent modifications) of proteins as they are expressed under native cellular conditions. Once a protein is translated from mRNA, the primary structure of the protein is often covalently modified through the action of enzymes. These modifications include the addition of a new moiety to the side chain of an amino acid residue, such as the addition of phosphate to a serine or proteolytic cleavage, such as removal of an initiator methionine or a signal sequence. Thus, the structural characterization of a protein includes both the linear organization of the amino acid sequence (as affected by alternative splicing and polymorphisms) and the presence of any post-translational modification that may arise within the sequence.

Mass spectrometry (MS) is an analytical technique that is used to identify unknown compounds, to quantify known compounds, and to ascertain the structure of molecules. A mass spectrometer is an instrument that measures the masses of ions that have been generated from individual molecules. This instrument measures the molecular mass indirectly, in terms of a particular mass-to-charge ratio of the ions. The charge on an ion is denoted by the fundamental unit of charge of an electron z, and the mass-to-charge ratio m/z is mass of the ion divided by its charge. For singly-charged ions, the m/z ratio is the mass of a particular ion in Da.

Tandem mass spectrometry (MS/MS) is a specific type of MS in which mass measurements of an intact ion and its constituent fragments are made in sequential steps. Generally in MS/MS, the intact mass of a protein ion is measured and the ion is isolated. Next, the instrument bombards ions of a sample with high intensity photons, electrons or neutral gas, breaking bonds, resulting in the formation of fragment ions from the molecular ions of the intact molecule. Although both positive and negative ions are generated with MS, only one polarity of an ion is detected with a particular instrumental set-up. Formation of gas phase sample ions allows the sorting of individual ions according to mass and their detection.

Living organisms are constantly synthesizing and degrading proteins. The degradation products of proteins are often found in various fluids of the organism, such as blood, urine, spinal fluid, cerebral spinal fluid, joints, saliva and serum. Many disease states include the production of an increased amount of a protein, the production of a protein form not normally produced, or a decrease in production of a protein. It is therefore possible to correlate the presence of the degradation products of proteins, also referred to as protein fragments or biomarkers, with disease states.

Precisely identifying biomarkers by MS, and deducing from which proteins they originated, presents significant challenges. Biomarkers are usually present in relatively low concentrations, which results in a low signal to noise ratio for the peaks in MS spectrum. Furthermore, this low signal to noise ratio usually results in fewer clearly identifiable fragment ions.

Generally, two approaches exist for analyzing biomarker proteins by MS. The conventional approach, termed the “bottom up” approach, relies upon digesting a given protein sample and analyzing the resultant protein digestion products by MS. The bottom up approach is labor-intensive, because one must discern the protein under analysis by piecing together disparate MS data obtained from individual sub-protein-sized peptide fragments that represent less than the entirety of the protein.

The second approach, termed the “top down” approach, relies upon direct MS analysis of intact protein as the protein exists in the proteome. The advantage to the top down approach is that MS analysis of intact proteins is robust, because one knows that the sample under MS analysis is an intact protein having a defined sequence from the genome. For at least this reason, the top down approach is gaining traction in the industry as the preferred way to conduct MS analysis of the proteome in the future. The perception in the field, however, is that a large amount of sample is required for performing a top down MS analysis of a sample. For this reason, whether justified or not, a widely-held perception in the field is that the top down approach is not presently amenable for conducting MS analysis of proteins from minute samples, such as clinical samples.

Thus, there is a need to provide new systems and methods for analyzing proteins using top down MS analysis that is scalable, high-throughput and robust to enable analysis of the proteome from clinical samples, preferably in the clinical setting. Toward this end, however, one must overcome the significant obstacles associated with sample processing for the top down approach to MS analysis of the proteome. The most significant obstacle to the top down approach of MS analysis of clinical samples is to obtain soluble intact proteins fractionated according to specific size(s).

A typical method of separating and analyzing the proteins is sodium dodecyl sulfate-polyacrylamide gel electrophoresis (SDS-PAGE), in which the proteins are separated due to a difference in selectivity by applying an electric field to a polyacrylamide gel. This method is a one-dimensional separation method that is widely used in the field of separating and identifying proteins in order of molecular weight and in the process of separating and identifying simple proteins, and has a problem in that the proteins have a tertiary structure denatured in an SDS solution, or are confined in the gel. The most serious limitation of SDS-PAGE relates to the recovery of protein from the gel. As an interfering compound, the presence of SDS may provide an impending limitation toward mass spectrometric (MS) analysis.

The aforementioned one-dimensional separation technology alone is insufficient to identify protein mixtures derived from the proteome. The generation, relative abundance, etc. of the protein are determined depending on an intracellular position or the physiological state of a cell or an organ, which must be separated initially and then properties of the protein mixtures are identified subsequently.

Thus, in order to separate the protein mixtures, a two-dimensional (2D) separation method called 2D-polyacrylamide gel electrophoresis (2D-PAGE) is used, in which the proteins, which are primarily separated according to the protein property, are secondarily separated according to the molecular weight of the protein [Zhou, F. et al., Anal. Chem. (2004) 76:2734-2740; Klose, J. et al., Electrophoresis (1995) 16:1034-1059; Righetti, P. G. et al., Anal. Chem. (2001) 73:320-326]. In this 2D-PAGE, the proteins are subjected to isoelectric focusing (IEF) in a narrow gel strip, i.e. an ampholyte carrier, in which a pH gradient is fixedly formed, according to isoelectric point (pI) of the ampholyte carrier. This process can require about 12 hours or more.

Subsequently, the gel strip is fixed on the upper end of a polyacrylamide gel plate in a transverse direction, and then the proteins are subjected to electrophoresis in a longitudinal direction. Thereby, the proteins are separated in the longitudinal direction according to the order of size, i.e. molecular weight. At this time, the proteins having low molecular weight move mainly to a lower end of the polyacrylamide gel plate. A total time required for the 2D-PAGE separation amounts to about 36 hours. After the 2D separation is terminated, protein spots shown on the polyacrylamide gel plate are dyed, thereby checking the number of proteins. The protein of each spot also can be recovered and identified using mass spectroscopy.

The 2D-PAGE is a labor-intensive method, is difficult to automate, and is limited in detection sensitivity and dynamic range. Further, separating the proteins without denaturation is difficult in the 2D-PAGE because the SDS solution is used to separate the proteins, which results in separation of the proteins in the denatured state, and recovering the sample is not easy because the separated proteins are also confined in a gel matrix. Accordingly, one must decompose the proteins in the 2D-PAGE gel using the enzyme, and recover the proteins in sub-protein-sized peptide form. Finally, the prolonged time requirements for processing proteome samples by the 2D-PAGE system precludes its use in high-throughput applications regardless of the type of MS analysis performed.

A capillary isoelectric focusing (CIEF) method is a method involving filling ampholyte carriers in silica capillaries along with proteins, applying an electric field to separate the proteins according to pI of the protein (Conti, M. et al., Electrophoresis (1996) 17:1485-1491). The technical aspects of IEF in CIEF method differ from that used in 2D-PAGE since CIEF uses silica capillaries rather than the gel strip. The CIEF method can be used to process protein samples in minute quantities due to the intracapillary separation and can be employed in high sensitivity applications to separate the proteins having a slight difference of 0.003 between their pI values (Quigley, W. C. et al., Anal. Chem. (2000) 76:4645-4658].

Notwithstanding these performance attributes, the CIEF method has limited fractionating capabilities, being applicable to samples of low to moderate protein complexity. The method is not amenable for fractionating samples having high protein complexity, such as that found in clinical samples encompassing complicated protein mixtures such as proteome. In order to increase separation efficiency, an attempt has recently been made to use the OFF in on-line connection with a secondary separation method such as chromatography rather than as a single analysis technique.

A typical example of the technology that carries out the 2D separation in on-line connection with the CIEF is CIEF-reversed phase liquid chromatography (RPLC) that connects the CIEF with the RPLC on line. The CIEF-RPLC secondarily separates proteins or peptide bands, which are separated by pI regions in the CIEF, in a chromatography column according to hydrophobicity difference between peptides (Chen, J. et al., Electrophoresis (2002) 23:3143-3148). With the use of this method, a result of conducting a test with peptide mixtures obtained by hydrolyzing the proteome of a fruit fly, Drosophila melanogaster, was that a peak capacity of more than 1800 could be obtained through separation of about 8 hours.

The CIEF-RPLC may be connected on line with a mass spectrometer using electrospray ionization (ESI) (Tnag, Q. et al., Anal. Chem. (1996) 68:2482-2487; Yang, L. et al., Anal. Chem. (1998) 70:3235-3241; Martinovi, S. et al., Anal. Chem. (2000) 72:5356-5360). However, because the ampholyte used for the CIEF separation is not removed, the CIEF-RPLC must be subjected to separate purification in order to remove the ampholyte after the OFF separation. As such, sample analysis is difficult in the CIEF-RPLC due to inhibition of ions in the solution without previous removal of the ampholyte. In order to solve this problem, the ampholyte must be considerably removed using membranes such as microdialyzable cathode cells (Zhou, F. et al., Anal. Chem. (2004) 76:2734-2740). Although the CIEF-RPLC is used to separate the proteins, denaturation of the proteins cannot be avoided due to use of an organic solvent during the RPLC separation, and the CIEF-RPLC cannot be applied to the separation of the proteins having large molecular weight.

In another system, CIEF-capillary gel electrophoresis (CGE), involves connecting the CIEF with the CGE on line to carry out separation in capillaries filled with a polyacrylamide gel instead of the aforementioned polyacrylamide gel plate according to molecular weight, and attempt to separate simple proteins such as hemoglobin (Yang, C. et al., Anal. Chem. (2003) 75:215-218). The CIEF-CGE is useful in separating peptide mixtures, which are obtained by hydrolyzing proteins with protein enzymes, rather than the proteins. However, due to protein chain breakdown occurring when the proteins pass through the chromatography column, protein loss within the chromatography column, and so on, it is difficult to apply the CIEF-CGE to the proteins.

As a different strategy to preparative gel electrophoresis, proteins can be eluted from the end of a gel column by continuous application of the electric field, wherein proteins are trapped by a molecular weight cut off (MWCO) membrane and subsequently collected. This technique is generally referred to as continuous elution tube gel electrophoresis. Although the ability to purify a target protein with extremely high resolution has been well-established, broad fractionation of an entire proteome with such methodology has been problematic. In general, systems based on this approach are biased toward lower MW proteins. Other significant limitations include long separation times and an unacceptably large dilution of sample during separation, particularly at high masses. These difficulties need to be overcome before continuous elution electrophoretic techniques can be generally adopted for comprehensive, broad mass range proteome separation

One attempt to solve this problem is the development of the separation technique GELFrEE (Gel-Eluted Liquid Fraction Entrapment Electrophoresis). The GELFrEE device provides for optimized conditions that enable a broad mass range proteome separation in a fast, effective, reproducible, and high-yield format. Referring to FIG. 1A,B, a prior art device employs a gel column 2 for electrophoretic separation along an applied electric field disposed between anode 1 and cathode 4. The proteins are ultimately eluted from the column and collected in the solution phase in a collection chamber 3 that is disposed between gel column 2 and cathode 4. See, for example, Tran, J. C. et al., Anal. Chem. (2008) 80:1568-1573.

Proteins are focused in a stacking gel section 5, fractionated according to size in resolving gel section 6, and eluted from the gel and subsequently confined in the collection chamber 3 for a defined time interval. Over the course of a run, as the migration rate decreases for larger proteins, the time interval for collection of subsequent fractions is simply increased to match the bandwidth of the larger MW proteins. Protein fractions are collected in an approximate “linear” MW profile, wherein the proteins remain focused during collection, ideally being recovered in single fractions over the entire mass range, and at consistently high yield. With the use of this device, an approximate 2-fold increase in concentration (relative to initial sample loading) can be maintained during sample collection.

A critical feature of the GELFrEE collection chamber 2 is the trapping efficiency of the 3.5 kDa MWCO membrane (not shown) located therein. A typical MWCO membrane, cellulose acetate, has an isoelectric point of 4.2, which would be negatively charged at an operating pH 8 to repel SDS-bound proteins. Yet one problem with use of membrane as trapping agents in collection chambers is that while SDS-bound proteins would be repelled from the membrane, SDS-free proteins substantially free of SDS could bind to the membrane, rendering the membrane-bound proteins unavailable in solution or having reduced efficiencies of recovery for subsequent processing.

Another example of the method of separating the proteins according to the order of molecular weight is flow field-flow fractionation (FIFFF), which is a type of field flow fractionation (FFF). The FIFFF is a separation analysis technique that is used for size- and shape-based separation of proteins, cells, water-soluble polymers, and nanoparticles, as well as property analysis of a diffusion coefficient, particle size, molecular weight, and so on etc. (Giddings, J. C. et al., Science (1976) 193:1244; Moon, M. H. et al., Anal. Chem. (1999) 71:2657). In the FFF-based separation, an available channel is a channel that has a cuboidal cross section and a hollow space. Further, because there is no stationary phase, samples are separated depending on the strength of an external field applied in a direction perpendicular to the flow of a fluid moving the samples along a channel axis. Thus, the FIFFF uses a cross flow of the fluid, and controls retention of macro-molecules such as proteins by regulating a flow rate of the cross flow.

The retention of the samples in the FIFFF channel is caused by a balance between the flow rate of the cross flow flowing out through a channel bottom and Brownian diffusion of the samples. In the case of the proteins, an average height of the samples moving in the channel is determined by a degree of the Brownian diffusion that varies depending on molecular weight or a Stoke's diameter. The smaller the molecular weight, the greater the diffusion. Thus, the diffusion and the flow rate of the cross flow are balanced at a position where the proteins are distant from the channel bottom. At this time, a separation flow flowing along a channel axis has a parabolic velocity profile, and the proteins and the macro-molecule samples are separated according to size. Accordingly, the samples having low molecular weight are discharged from the channel, so that the samples are separated according to the size of molecular weight.

FIG. 2 shows a typical prior art configuration of an asymmetrical flow field-flow fractionation (AFIFFF or AF4) system applied to separation of proteins. The AF4 device is composed of an upper block 7 and a lower block 8 that define the depletion wall and accumulation wall, respectively. Disposed between the upper block 7 and lower block 8 is a spacer 9 having a cut-out 10 and a membrane 11. The AF4 channel is defined by a cut-out 10 within spacer 9. As explained in greater detail below, the shape of the cut-out 10 defines critical performance parameters of the AF4 channel. This channel has an asymmetrical channel structure in which only a lower block 8 under the channel has a frit 12, unlike a conventional symmetrical channel structure in which upper and lower blocks of a channel have respective frits. A fluid is transferred from a high performance liquid chromatography (HPLC) pump 18, and protein samples separated and eluted in the channel at outlet 15 are detected using an ultraviolet/visible radiation (UV/VIS) detector (not shown). The proteins are separated in the AF4 channel as follows. The protein samples injected into the channel at inlet 13 through an injector 16 are subjected to a sample relaxation-focusing process before the separation is initiated. This sample relaxation-focusing process serves to put the samples in equilibrium between the strength of an external field applied outside the samples and diffusion of the samples, and is an essential process for the AF4 device when used as a high resolution separation device. The sample relaxation-focusing is carried out by injecting the fluid through an inlet 13 of the channel by action of pump 18 as well as an outlet of the channel or an inlet 14 of a focusing flow by action of pump 17 as in FIG. 2 to adjust a ratio of flow rates of the two flows such that the samples injected into the channel can be focused at a position corresponding to a triangular base of the channel inlet. Usually, this sample relaxation-focusing is experimentally applied by computing a ratio of a position where the samples are subjected to relaxation and focusing to an entire length of the channel to calculate the flow rate ratio. For example, the flow rate ratio may be finally determined by injecting a material such as organic dye, water-soluble ink, or the like to check a position where the material is focused. The sample relaxation-focusing requires a sufficient time for a buffer solution corresponding to a volume of the channel to flow out through the channel bottom. When the sample relaxation-focusing process is completed, inflow of the focusing flow is interrupted at inlet 14, and the fluid is transferred only to the channel inlet 13. At this time, a ratio of a cross flow flowing out through the channel bottom 8 to an outlet flow transferred to a detector is adjusted. Thereby, the proteins are separated. In the case where the focusing flow is adjusted in such a manner that a part thereof can flow into the detector during the sample relaxation-focusing and be transferred by the flow rate of the outlet flow used when the proteins are separated, a phenomenon in which the flow of the fluid comes to a standstill at the detector can be avoided when the relaxation-focusing process is transited to the separation process.

In the FIFFF using the asymmetrical channel, when the proteins are separated, the proteins can be separated in order from low molecular weight to high molecular weight. Because the separation solution uses the buffer solution, the proteins are separated without denaturation. Because no filler is filled in the channel, risks such as breakdown of the protein samples or blocking of the separation channel can be minimized. When thickness and width of a spacer 9 determining the volume of the channel are adjusted, the flow rate of the fluid, separation efficiency, etc. can be varied, and the proteins can be separated at a micro flow rate, which is suitable to separate a very small amount of proteins (Kang, D. et al., Anal. Chem. (2004) 76:3851-3855; Oh, S. et al., J. Separation Sci. (2007) 30:1982-1087).

In comparison with the gel electrophoresis, the separation method using the AF4 device can be used to separate the proteins in order from low molecular weight to high molecular weight, and minimize the breakdown of the protein samples or the blocking of the separation channel. However, the separation method using the AF4 device is not very high in separation capability, and has difficulty in carrying out the separation on the basis of various properties of the protein.

As a plan to overcome this problem, a 2D separation method, CIEF-Hollow fiber flow-field flow fractionation (HF FIFFF) has been developed, whereby isoelectric focusing and molecular weight based separation without using the gel are possible. The CIEF-HF FIFFF is configured to serially connect HF FIFFF with the CIEF method that carries out the isoelectric focusing in the capillaries, and more particularly, fills the ampholyte carriers in the silica capillaries along with the proteins, and applies the electric field to separate the proteins according to the pI of the protein (Conti, M. et al., Electrophoresis (1996) 17:1485-1491). The HF FIFFF belongs to another example of separating and analyzing the proteins, and is a separation method that uses a hollow fiber membrane as a separation channel (Lee, W. J. et al., Anal. Chem. (1999) 71:3446; Moon, M. H. et al., J. Microcolumn (1999) 11:676; Reschiglian, P. et al., Anal Chem. (2005) 77:47). In the HF FIFFF, the function of an external field is determined by the flow rate of a cross flow or a radial flow discharged to an outer wall of the hollow fiber membrane, and samples in the channel maintain an equilibrium with the external field. In this case, the samples proceed in the shape of a circular band. At this time, a ratio of the flow of the samples to a separation flow moving toward a longitudinal axis of the channel is adjusted, thereby adjusting a separation speed.

The CIEF-HF FIFFF (Kang, D. et al., Anal. Chem. (2006) 78:5789-5798) has an advantage in that the proteins can be separated in two dimensions without using a gel. One disadvantage to their technique is the amount of separable proteins is restricted due to the limited capacity of the capillary during isoelectric focusing. Further, while a fraction of the proteins separated according to pI after the primary separation is transferred to a hollow fiber module, and then separated according to molecular weight, the other proteins stand by in the capillaries, and then are separated in the hollow fiber module. For this reason, a total separation time is prolonged by a desired fraction of pI. In addition, while some of the protein samples primarily separated according to pI are secondarily separated in the hollow fiber module according to the molecular weight, the other pI fractions must stand by in the capillaries under the electric field. In this process, the proteins are slightly shifted due to the influence of an electroosmotic flow driven on inner walls of the capillaries. This results in a problem that allows the separated fractions to be mixed again, and causes contamination of the fractions. In the case of the CIEF-HF FIFFF, because a maximum amount of the proteins that can be injected at once is about 40 μg, there is the limitation of a capacity to some extents to process a large amount of proteome samples.

Moon et al. disclosed in U.S. Pat. No. 8,298,394 a modified AF4 device that includes at least one fluid channel having a predetermined length. Each fluid channel is divided into two sections: an isoelectric focusing section that primarily separates proteins from protein samples according to isoelectric point (pI), and a flow field-flow fractionation section that secondarily separates the primarily separated proteins according to molecular weight. The isoelectric focusing sections of the fluid channels are connected with each other. The modified AF4 device of Moon et al. is characterized in that the proteins primarily separated according to pI of the protein can be secondarily separated and analyzed through multiple channels capable of simultaneously driving the flow field-flow fractionation sections that separate the primarily separated proteins according to order of molecular weight. The modified AF4 device of Moon et al. is an isoelectric focusing-flow field-flow fractionation multi-channel apparatus, and may be called a multi-channel apparatus for non-gel based two-dimensional (pI and molecular weight) protein separation when characteristics of the protein separating apparatus are intended to be expressed intensively. Further, the modified AF4 device of Moon et al. includes a feature that can naturally remove an ampholyte through a semi-permeable membrane installed on a multi-channel bottom in the process of carrying out flow field-flow fractionation because isoelectric focusing of the proteins is carried out in the multiple channels where the flow field-flow fractionation are carried out. Thus, even the protein samples separated through a conventional capillary isoelectric focusing technique do not require a separate re-analysis process for removing analysis obstacle factors caused by the ampholyte obtained together in the mass analysis process of the proteins, so that a time to carrying out the protein separation process can be reduced.

Moreover, the modified AF4 device of Moon et al. requires neither a separate connection tube for transferring the samples after the isoelectric focusing because the isoelectric focusing and the molecular weight based separation are sequentially carried out in the multiple channels, nor use an organic solvent for the separation, thereby avoiding denaturation of the proteins when the protein samples are separated. The modified AF4 device of Moon et al. can separate the protein samples to be analyzed according to pI and molecular weight regions, particularly in a liquid phase rather than in a gel phase using a combination of pI-based and molecular weight based separations, collect protein fractions of desired pI-molecular weight regions, and separate a large quantity of protein samples and proteins by increasing a processing rate as needed. In addition, in the process of carrying out molecular weight based separation on the proteins separated according to pI, protein bands of several pI regions can be independently and simultaneously subjected to the molecular weight based separation without waiting of the proteins of other pI regions when the molecular weight based separation is performed on the proteins of any pI region.

The modified AF4 device of Moon et al. can be used as part of an on-line system to separate proteins according to molecular weight range, converts the collected protein fractions into peptides through enzyme treatment amenable for a bottom up MS analysis, separates the peptides using nanoflow liquid chromatography-electrospray ionization-tandem mass spectrometry (LC-ESI-MS-MS), and identifies the proteins through comparison of a mass spectrum with a protein database.

A critical aspect of AF4 device performance is the relationship between channel design and separation efficiency. Moon and coworkers studied the separation efficiencies of three different AF4 channel designs using polystyrene latex standards. See Ahn, J. Y. et al., J. Chromatogr. A (2010) 1217:3876-3880. Channel breadth was held constant for one channel (rectangular profile), and was reduced either linearly (trapezoidal profile) or exponentially (exponential profile) along the length for the other two. The effective void volumes of the three channel types were designed to be equivalent. Theoretically, under certain flow conditions, the mean channel flow velocity of the exponential channel could be arranged to remain constant along the channel length, thereby improving separation in AF4. Particle separation obtained with the exponential channel was compared with particle separation obtained with the trapezoidal and rectangular channels. Moon and coworkers demonstrated that at a certain flow rate condition (outflow/inflow rate=0.2), the exponential channel design indeed provided better performance with respect to the separation of polystyrene nanoparticles in terms of reducing band broadening. While the trapezoidal channel exhibited a little poorer performance than the exponential, the strongly decreasing mean flow velocity in the rectangular channel resulted in serious band broadening, a delay in retention time, and even failure of larger particles to elute.

Several groups have used AF4 techniques to study protein separations. See for example, Lee, J. Y. et al., J. Chromatog. A (2011) 1218:4144-4148; Kim, J. Y. et al., Anal. Chem. (2012) 84:5343-5350; Yohannes, G. et al., Anal. Biochem. (2006) 354:255-265; A. Litzén, A. et al., J. Chromatog. A (1989) 476:413-421; and Wahlund, K. G. et al., Anal. Chem. (1987) 59:1332-1339. These studies were carried out with conventional or large-scale AF4 techniques and UV absorption detectors. Only one reference (Yohannes et al.) shows data with a small AF4 channel, but that experiment was carried out a thick channel (500 μm) for high resolution. Therefore, relatively high flow rate was applied (outflow=2.4 mL/min) for reducing a retention time, rendering an AF4 device with this dimension unacceptable to obtain an electrospray ionization of proteins.

The removal of matrix from sample solution by an AF4 channel was applied in analysis of soil extracts (Sangsawong, S. et al., Spectrochimica Acta Part B: Atomic Spectroscopy (2011) 66:476-482). The technique is based on a general AF4 system, wherein a conventional AF4 channel was used to fractionate analytes and to remove metal ions from solution. An inductively coupled plasma optical emission spectrometry (ICP-OES) was used for analysis of metal ions such as Cu, Mn, Pb, and Zn. In this technique, metal ions make the complex with poly (ethylene imine) and only Mg²⁺ ions are removed through membrane pores by a cross flow action. A conventional AF4 channel with a typical condition was used (\acute{V}ᵢₙ/\hat{V}ₒᵤₜ=3.0/1.0 mL/min.) because ICP-OES can be operated in higher flow rate. Besides the unacceptably high flow rates used in this device are not amenable for electrospray ionization of proteins, the previous technique is unavailable to automation and most sample preparation step should be carried out prior to analysis.

### SUMMARY

In a first respect, a system for analyzing a specimen containing the proteome by mass spectrometry is disclosed. The system includes a protein separation module; a matrix processing module; and a mass spectrometer module. The protein separation module, the matrix processing module and the mass spectrometer are in fluid communication with one another.

In a second respect, a subsystem for preparing a specimen containing the proteome for analysis by mass spectrometry is disclosed. The subsystem includes a protein separation module, and a matrix processing module. The protein separation module and the matrix processing module are in fluid communication with one another.

In a third aspect, a method of analyzing a specimen containing the proteome by mass spectrometry is disclosed. The method includes several steps. The first step is injecting a protein extract obtained from the specimen into a protein separation module to separate the protein extract into protein fractions having discrete molecular masses. The separation is effected using a continuous SDS-PAGE gel column and the protein fractions having discrete molecular masses are eluted in solution form the continuous SDS-PAGE gel column and collected into a collection chamber having a negatively charged acrylamide trap. The second step is flowing the collected protein fractions having discrete molecular masses into a matrix processing module in fluid communication with the protein separation module to produce a matrix-free, protein-containing eluate. The matrix processing module comprises an AF4 device having a miniaturized channel with one of the following properties: (a) a length in the range from about 3.0 cm to about 10.0 cm, a width in the range from about 0.20 cm to about 1.00 cm and a height (thickness) in the range from about 10 microns to about 300 microns; or (b) a volume in the range from about 3×10⁻⁶ cm³ to about 1.5×10⁻³ cm³, and a surface area in the range from about 0.30 cm² to about 5.00 cm². The matrix processing module is configured to remove matrix components that interfere with mass spectrometry analysis of proteins. The third step is flowing the matrix-free protein-containing eluate into a mass spectrometer in fluid communication with the matrix processing module to analyze protein.

These and other features, objects and advantages of the present invention will become better understood from the description that follows. In the description, reference is made to the accompanying drawings, which form a part hereof and in which there is shown by way of illustration, not limitation, embodiments of the invention.

### BRIEF DESCRIPTION OF THE DRAWINGS

FIG. 1A depicts an embodiment of a prior art GELFrEE device having an anode (1), gel column (2), collection chamber (3) and cathode (4).

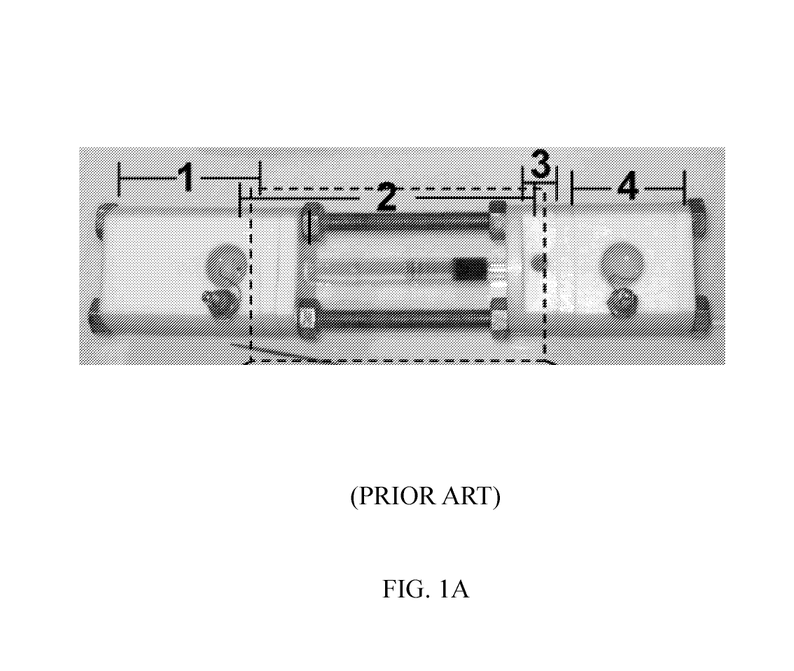

FIG. 1B shows a close-up of the gel in gel column (3) of FIG. 1A that shows the stacking gel section (5) and resolving gel section (6), wherein a prestained MW protein ladder is depicted in the resolving gel section (6).

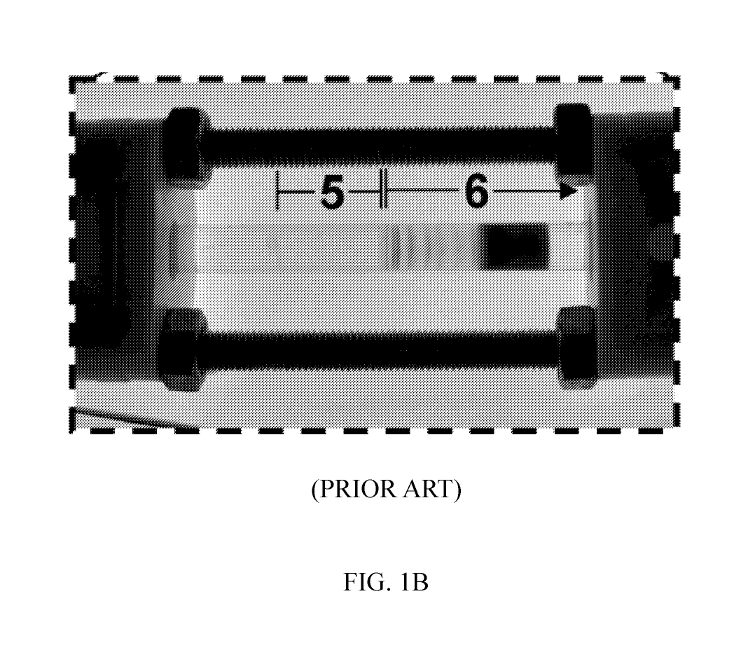

FIG. 2 depicts a prior art embodiment of an AF4 device.

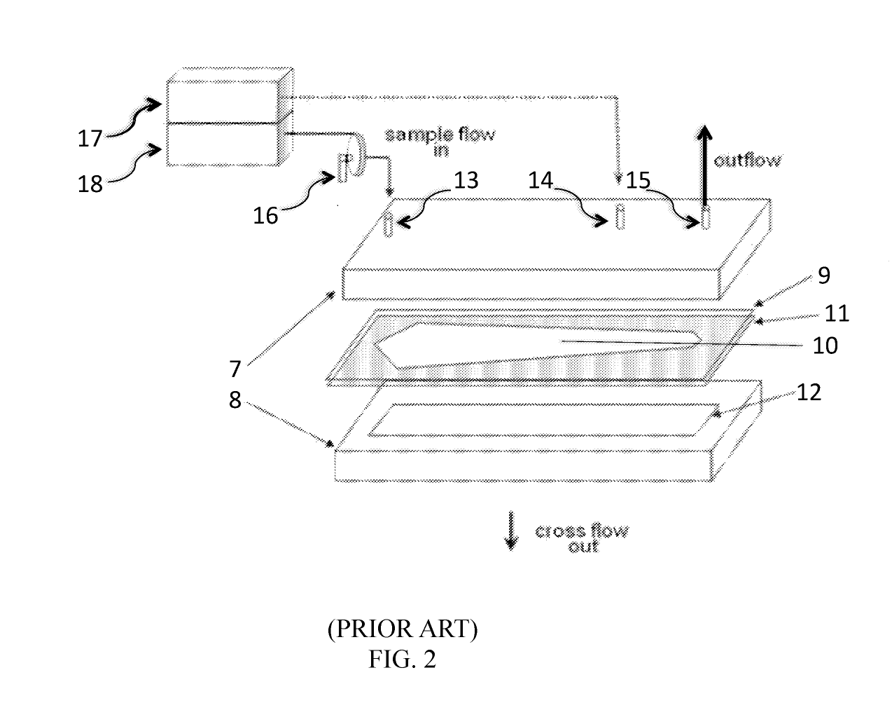

FIG. 3 depicts a schematic view of one embodiment of the device.

FIG. 4 depicts a schematic view of a miniaturized continuous tube gel electrophoresis device.

FIG. 5 depicts a gel image of fractions collected using the device described in FIG. 4.

FIG. 6 depicts a gel image showing the effective in solution digestion of fractions collected from gel electrophoresis prior to SDS removal.

FIG. 7 depicts a schematic of one embodiment enabling auto-sampling of fraction collection from gel electrophoresis.

FIG. 8 depicts a gel image of fractions collected from gel electrophoresis using an auto-sampler.

FIG. 9A depicts total ion chromatogram of running buffer (0.1% SDS, Tris-Glycine) treated with peptides with weak anion exchange resin.

FIG. 9B depicts total ion chromatogram of water with peptides treated with weak anion exchange resin.

FIG. 10A depicts an exemplary total ion chromatogram showing similarity in SDS removal efficacy using the classical precipitation method with acetone/urea. The number of protein identifications achieved from a 12 fraction run was 1308 and the false discovery rate was 1.0%.

FIG. 10B depicts an exemplary total ion chromatogram showing similarity in SDS removal efficacy using in-solution HiPPR™. The number of protein identifications achieved from a 12 fraction run was 1360 and the false discovery rate was 1.3%.

FIG. 10C depicts an exemplary total ion chromatogram showing similarity in SDS removal efficacy using in-solution anion exchange. The number of protein identifications achieved from a 12 fraction run was 1317 and the false discovery rate was 1.3%.

FIG. 11 depicts a schematic of one embodiment enabling SDS removal using proprietary Piece SDS removal resin.

FIG. 12 depicts a schematic diagram of an exemplary embodiment of the disclosed AF4 device.

FIG. 13 illustrates a photograph of an exemplary embodiment of the disclosed AF4 device.

FIG. 14A depicts an exemplary channel for a conventional AF device defined by a spacer having dimensions of 1.5˜2.5 cm (width), 25˜30 cm (length) and 250˜300 microns (thickness).

FIG. 14B depicts an exemplary miniaturized channel defined by the spacer having dimensions of about 0.5 cm (width), about 5 cm (length) and about 170˜200 microns (thickness) for use within the interior of a preferred embodiment of an AF4 device for coupling to mass spectrometry.

FIG. 15 illustrates the principle of reduced protein retention by using a thin channel spacer. Each peak number represent 1: carbonic anhydrase (29 kDa), 2: BSA, 3: BSA dimer. Flow rate conditions were an inflow rate of 0.5 mL/min and a outflow rate of 0.05 mL/min.

FIG. 16A illustrates the principle of reduced retention time by decreasing the effective channel length from a length of 6 cm (black chromatogram tracing) to a length of 3.5 cm (red chromatogram tracing) for carbonic anhydrase (29 kDa) (peak 1), BSA (peak 2) and BSA dimer (peak 3). Flow rate conditions were an inflow rate of 0.5 mL/min and a outflow rate of 0.05 mL/min.

FIG. 16B depicts the channel designs used in FIG. 16A, wherein the thickness of each channel was fixed to 178 microns (0.007″).

FIG. 17A depicts mass spectral analysis of myoglobin protein fractions dissolved in water only, wherein total ion count of 2×10⁶ species was detected.

FIG. 17B depicts an exemplary total ion chromatogram of myoglobin before SDS removal using an exemplary embodiment of the modified AF4 device on-line with the disclosed system.

FIG. 17C depicts an exemplary mass spectral analysis of myoglobin protein fraction after 0.1% SDS removal using an exemplary embodiment of the modified AF4 device on-line with the disclosed system.

FIG. 17D depicts an exemplary mass spectral analysis of myoglobin protein fraction after 0.6% SDS removal using an exemplary embodiment of the modified AF4 device on-line with the disclosed system.

FIG. 18 depicts the mass spectrum of the intact ferritin fraction. Large and small peak was from an intact monomer and dimer of ferritin complex, respectively.

FIG. 19A depicts an exemplary total ion chromatogram for SDS removal using an exemplary embodiment of the modified AF4 device on-line with the disclosed system, wherein different protein fractions were sampled at retention times ˜2 min, ˜4 min and ˜6 min and then subjected to mass spectrometry (see FIGS. 19B, C, and D, respectively).

FIG. 19B depicts an exemplary mass spectral analysis for a protein fraction sampled at ˜2 min as in FIG. 19A.

FIG. 19C depicts an exemplary mass spectral analysis for a protein fraction sampled at ˜4 min as in FIG. 19A.

FIG. 19D depicts an exemplary mass spectral analysis for a protein fraction sampled at ˜6 min as in FIG. 19A. The asterisks indicate exemplary mass ions bearing an SDS adduct.

FIG. 20A depicts an exemplary mass spectrum for a carbonic anhydrase diluted in SDS-containing buffer (1.0 μg of injection amount), subjected to a 5 minute focusing step and 0.05 mL/min flow rate.

FIG. 20B depicts an exemplary mass spectrum for a carbonic anhydrase diluted in SDS-containing buffer (1.0 ∞g of injection amount), subjected to a 5 minute focusing step and 0.10 mL/min flow rate.

FIG. 20C depicts an exemplary mass spectrum for a carbonic anhydrase diluted in SDS-containing buffer (1.0 μg of injection amount), subjected to a 5 minute focusing step and 0.20 mL/min flow rate.

FIG. 20D depicts an exemplary mass spectrum for a carbonic anhydrase diluted in SDS-containing buffer (1.0 μg of injection amount), subjected to a 5 minute focusing step and 0.30 mL/min flow rate.

FIG. 20E depicts a plot of relative ratio between intact protein (+27 ion (m/z 1075.7); diamonds) and +1 SDS adduct (squares) and +2 SDS adduct (triangles) as a function of flow rate, as extracted from FIGS. 20A-D.

FIG. 21A depicts an exemplary mass spectrum of dodecyl sulfate anion following negative mode focusing at a flow rate of 0.05 mL/min for 5 min.

FIG. 21B depicts an exemplary plot of absolute peak intensity of dodecyl sulfate ion as a function of flow rate following focusing for the experiments performed with negative mode focusing.

FIG. 22A depicts an exemplary mass spectrum of carbonic anhydrase in carrier medium containing 30% acetonitrile-0.1% formic acid without added ammonium bicarbonate. Sample injection amount was 10 μL and the focusing step was 0.3 mL/min for 3 minutes.

FIG. 22B depicts an exemplary mass spectrum of carbonic anhydrase in carrier medium containing 30% acetonitrile-0.1% formic acid-15 mM ammonium bicarbonate. The sample amount and focusing step were the same as in FIG. 22A.

FIG. 22C depicts an exemplary mass spectrum of carbonic anhydrase in carrier medium containing 30% acetonitrile-0.1% formic acid-15 mM ammonium bicarbonate. The sample amount and focusing step were the same as in FIG. 22A.

FIG. 22D depicts a plot of the relative MS intensities of protein peak ion (diamonds) and its corresponding SDS adduct (squares) as a function of ammonium bicarbonate concentration in the carrier medium.

FIG. 22E depicts a plot of the relative MS intensity of the SDS adduct to its protein peak ion as a function of ammonium bicarbonate concentration in the carrier medium based upon data presented in FIG. 22D.

FIG. 23A depicts an exemplary spacer design having a length of about 3 cm (tip to tip) and an effective reduction in volume by 1:3 of the spacer design presented in FIG. 14B.

FIG. 23B depicts an exemplary mass spectrum for a protein sample processed at an outflow rate of 5.0 μL/min using an AF4 device fitted with the spacer design of FIG. 23A.

FIG. 23C depicts an exemplary mass spectrum for a protein sample processed at an outflow rate of 2.5 μL/min using an AF4 device fitted with the spacer design of FIG. 23A, wherein the signal to noise ratio for mass spectral sensitivity was increased about 3-fold relative to that observed in FIG. 23B.

FIG. 24A depicts an exemplary mass spectrum for BSA (1.0 μg) standard protein in a carrier solution of water/acetonitrile/formic acid (90.0/9.9/0.1%) subjected to processing via an AF4 device fitted with a spacer composed of PET.

FIG. 24B depicts an exemplary mass spectrum for BSA (1.0 μg) standard protein in a carrier solution of water/acetonitrile/formic acid (90.0/9.9/0.1%) subjected to processing via an AF4 device fitted with a spacer composed of PEEK.

FIG. 25A depicts an exemplary embodiment of an AF4 device. The cross-sectional perspective denoted by the orange box is presented in FIG. 25B.

FIG. 25B depicts a cross-sectional perspective of the AF4 device illustrated in FIG. 25A, showing in greater detail the location of intended crossflow through the device (illustrated by the highlighted oval region) in relation to device sub-components.

FIG. 25C depicts another cross-sectional perspective similar to FIG. 25B, showing where gaps (denoted by A and B) can contribute to leakage in the device.

FIG. 26A depicts another embodiment of an AF4 device, where the inclusion of modified bottom plate 202 and a PEEK tape-based spacer 207 provides for an improved leakage-free design.

FIG. 26B depicts an exemplary prototype of a bottom plate 202 as characterized in FIG. 26A, wherein locations of the crossflow outlet port 202A, O-ring 203A and frit 205 are illustrated.

FIG. 26C depicts an exemplary prototype of a top plate 201 as characterized in FIG. 26A, wherein locations of the spacer 207 and one of the plurality of flow inlets 209 are illustrated.

FIG. 27A depicts an exemplary SDS-PAGE slab gel image of the GELFrEE fractionation of 3 standard proteins, carbonic anhydrase (#7), myoglobin (#4), and ubiquitin (#1). Representative mass spectra of carbonic anhydrase (#7), myoglobin (#4), and ubiquitin (#1) are illustrated in FIGS. 27B, 27C and 27D, respectively.

FIG. 27B depicts an exemplary mass spectrum of carbonic anhydrase (#7) following online clean up on the prototype AF4 device 200 illustrated in FIG. 26.

FIG. 27C depicts an exemplary mass spectrum of myoglobin (#4) following online clean up on the prototype AF4 device 200 illustrated in FIG. 26.

FIG. 27D depicts an exemplary mass spectrum of ubiquitin (#1) following online clean up on the prototype AF4 device 200 illustrated in FIG. 26.

FIG. 28A depicts the protocol used to compare recovery between methanol-chloroform precipitation and a method using the in-line removal platform.

FIG. 28B depicts a representative histogram of the relative peak areas of the indicated protein standards following precipitation/resuspension (red bars) or the in-line removal platform (blue bars). Carbonic anhydrase showed the lowest recovery following the precipitation method, yielding only 10.1% of the signal achieved with the in-line removal platform. The other proteins also showed low recovery (15˜55%) after the precipitation/resuspension step that appears to be protein dependent.

FIG. 29A depicts an exemplary SDS-PAGE slab gel visualization of the GELFrEE fractionation of an acid extract derived from HeLa S3 cells. Three strong bands were observed, corresponding to the members of the histone family, H4 (fraction #2), H2B (fraction #4) and H3.3 (fraction #6). Each fraction was treated with in-line detergent removal, generating the three ion trap mass spectra shown in FIGS. 29B, 29C and 29D.

FIG. 29B depicts an exemplary ion trap mass spectrum for fraction #2, which corresponds to histone H4 (11306.3Da).

FIG. 29C depicts an exemplary ion trap mass spectrum for fraction #4, which corresponds to histone H2B (13969.1Da).

FIG. 29D depicts an exemplary ion trap mass spectrum for fraction #6, which corresponds to histone H3.3 (15370.7Da).

FIG. 29E depicts an exemplary mass spectrum obtained from a histone fraction from GELFrEE (fraction #7 from FIG. 29A) following in-line detergent removal acquired with a Fourier-Transform mass spectrometer. The insets show high-resolution mass spectra of the isotopically resolved species corresponding to the highest peak of each of the four core histone fractions shown above the principal mass spectrum. The adjacent isotopic clusters in a same group are proteoforms corresponding to differential acetylation and methylation. In the case of H2A and H2B, the adjacent proteoforms are due to different members of these gene family members No evidence for SDS adduction could be observed in these data.

FIG. 30A depicts an exemplary schematic diagram of a preferred on-line separation and cleanup system. A prototype protein separation module is attached to matrix processing module with 6-port valve and sample loop enabling for bypass capabilities.

FIG. 30B depicts an exemplary prototype gel-based protein separation module and its separation with pre-stained molecular weight marker. The buffer reservoir and hand-casted gel column was homemade and all parts were connected with general HPLC fittings.

FIG. 31A depicts an exemplary extracted (+8 ion of ubiquitin) fractogram of ubiquitin.

FIG. 31B depicts an exemplary MS1 spectrum at the center of a peak of the fractogram of FIG. 31A. Determination of the molecular weight of the protein, ubiquitin, was accomplished by measurement of the isotopic distribution with high-resolution mass spectrometry.

FIG. 31C depicts an exemplary extracted (top 5 peaks of myoglobin) fractogram of myoglobin.

FIG. 31D depicts an exemplary MS1 spectrum at the center of a peak of the fractogram of FIG. 31C.

FIG. 31E depicts an exemplary extracted (top 5 peaks of carbonic anhydrase) fractogram of carbonic anhydrase.

FIG. 31F depicts an exemplary MS1 spectrum at the center of a peak of the fractogram of FIG. 31E. Some of myoglobin ions were also observed by overlapping in same fraction containing carbonic anhydrase.

### DETAILED DESCRIPTION

Apparatus systems and sub-systems are disclosed that provide for on-line processing of small proteome samples, beginning with native specimens, such as those obtained from a patient in a clinical setting, to yield diagnostic MS analysis of discrete proteins from the native specimens. The apparatus system and methods disclosed herein provide a number of advantages over prior art apparatuses and methods of mass spectrometric analysis of biomolecules following a separation step. First, the disclosed apparatus directly couples separation of biomolecules to removal of the separation medium. Second, the disclosed apparatus and method may optionally provide for concentration of biomolecules concurrently with isolation. Third, the disclosed apparatus and method provide for high recovery of biomolecules by using surfactant medium. Fourth, the disclosed apparatus and method provide an optional method for online protein digestion, a method for removal of surfactant, salt or other interfering agents that can affect mass spectrometric analysis. Fifth, the fractions can be directly introduced online into mass spectrometry through nanospray or MALDI. Sixth, the disclosed apparatus and method is fully automatable, thereby allowing user-friendly access of robust MS diagnostic technology to non-expert users, such a staff and technicians in a clinical setting.

### System and Assembly

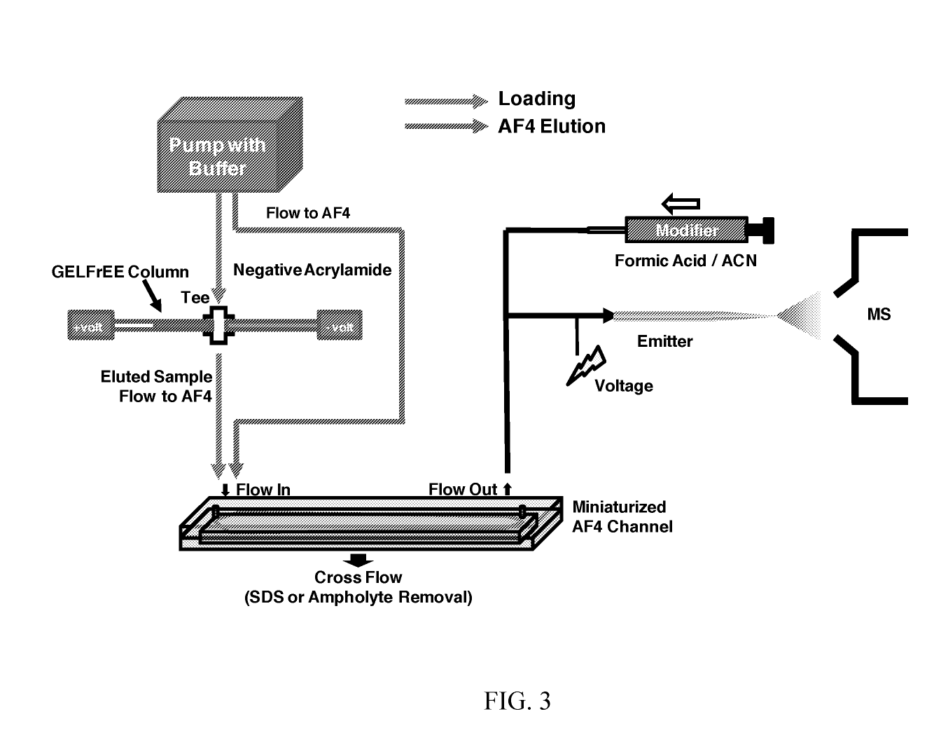

An overview of one embodiment of a fully assembled system is depicted in FIG. 3. The system comprises a protein separation module, a matrix processing module and a mass spectrometer module, wherein the aforementioned modules are in fluid communication. Focusing on the protein separation module, the usual anode, and cathode chambers are assembled at the ends with a separation column joined to a negatively charged medium by a tee. The tee interface is connected to a syringe pump that controls fluid mobility into an on-line matrix processing module where the matrix used during generation of the fractions in the protein separation module are removed from the obtained fractions. The on-line matrix processing module can be any device that provides removal of matrix components, such as SDS and other MS-interfering contaminants, from the fractions and include preferably an AF4 device or ion exchange resin. A preferred on-line matrix processing module is an AF4 device, the details of which are presented below. In other embodiments, the matrix processing module can be an ion exchange resin or one of several commercially available resins (see Examples 5 and 6). The fraction-containing eluent, after passing through an AF4 device or ion exchange resin, will be cleared of SDS and can thereafter be introduced online to a mass spectrometry module for performing nanospray mass spectrometry.

Alternatively, the post-processing module fractions can be introduced through MALDI using a heated droplet interface as described in Patent Application 20090302208. A separate syringe pump and valve system can be used to facilitate this process. In a different embodiment, the gel electrophoresis component of the invention can be comprised of radial gel electrophoresis.

### Protein Separation Module

A. SDS Gel Electrophoresis.

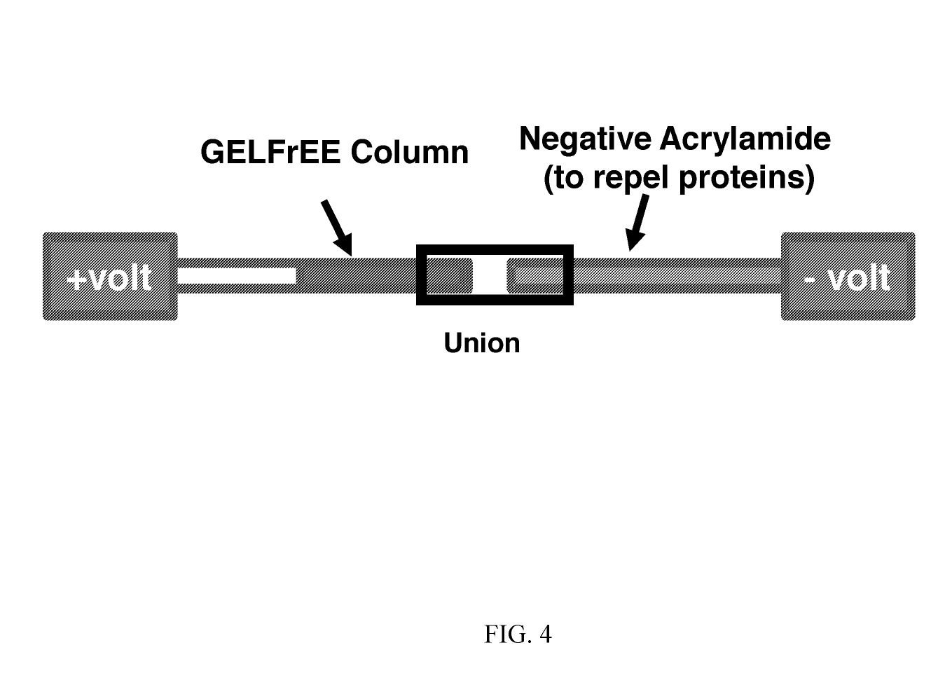

The protein separation module includes a 1-D SDS-containing gel electrophoresis device. The dimensions of the SDS-containing gel electrophoresis device can be varied. Column diameters of as large as 4 inches to as low as 1 mm have been tested. Therefore, the electrophoresis separation system described herein encompasses all continuous elution gel electrophoresis setups including but not limited to capillary gel electrophoresis, GELFrEE, RADGEL, preparative gel electrophoresis, etc. The disclosed SDS-containing gel electrophoresis device is presented on a miniaturized scale to ensure compatibility with the down-stream protein separation module that is in fluid communication with the SDS-containing gel electrophoresis device. Because the SDS-containing gel electrophoresis device is configured on a miniaturized scale, an entirely different design from that used in prior art GELFrEE devices is employed since the usual collection chamber fitted with a membrane trap of the prior art GELFrEE devices will result in significant loss at the low volumes of processed samples contemplated herein. The separation column is preferably a 1 mm i.d. SDS-PAGE column cast in PEEK tubing (FIG. 4). The membrane trap replaced by another equivalent SDS PAGE column that is preferably co-polymerized with an acidic compound having pKa 3.5. A preferred acidic compound has a chemical composition CH₂═CH—CO—NH—R, where R denotes any weak acidic group with pKa 3.6 such as a carboxyl. One preferred acidic compound is available under the trade name Immobiline™ from Sigma-Aldrich and is exclusively used in isoelectric focusing. The acidic compound covalently binds to the bis-acrylamide polymer that, at the normal GELFrEE pH conditions, will result in a negatively charged polymer.

Replacing the cutoff membrane of the prior art device with a negatively charged acrylamide has never been performed in any preparative gel electrophoresis setups and this innovation replaces the traditional molecular weight cutoff filter with a negatively charged acrylamide gel that prevents the proteins from migrating into the anode chamber.

The separation media can include any media useful for separating biomolecules. The media includes, without limitation, media that separates biomolecules on the basis of the size or mass of the molecules. These include, without limitation, gel filtration materials, such as sephadex and sepharose and materials for electrophoretic separations based on molecular size, including polyacrylamide and agarose gels. Other separation media include ion exchange materials, including cation and anion exchange resins, affinity materials, including general affinity materials such as phosphocellulose, hydroxyapatite and blue dextran. More specific affinity materials are also included, such as materials carrying a ligand to which certain biomolecules bind. The ligands include, without limitation, antibodies, proteins, peptides, nucleic acid sequences, carbohydrates, and other ligands. The separation media can also include hydrophobic and hydrophilic materials that separate biomolecules on the basis of hydrophobicity.

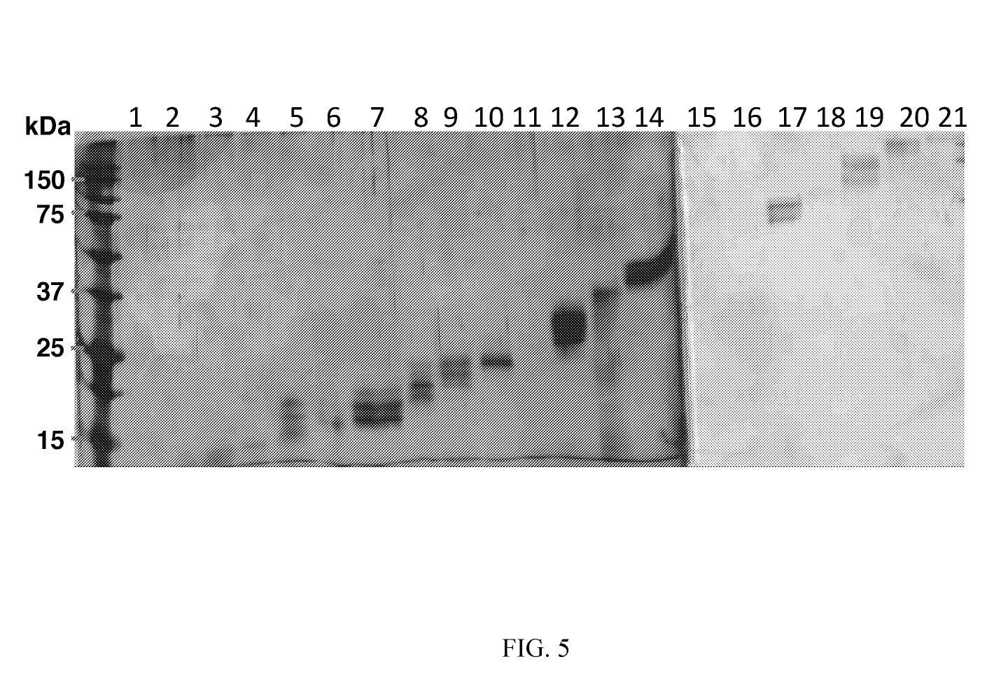

The collection chamber can be a simple Teflon sleeve into which the two gel columns are inserted. A gel image depicting the resolution achieved in this column system (i.d. of 1 mm) is shown in FIG. 5. The loading capacity for this preferred embodiment is about 10 μg.

While the separation resolution was encouraging, the fraction collection is tedious since it involves removing the sleeve, and significant loss can be expected in this process. With such small dimensions, it is more suitable to collect the fractions in an automated fashion by introducing a buffer flow into the collection chamber to capture and mobilize proteins. One preferred embodiment provides an interface by replacing the Teflon union with a Tee or cross where the pumped buffer will elute into and out of the inlet and outlet of the ports (see Example 4).

B. Online Digestion

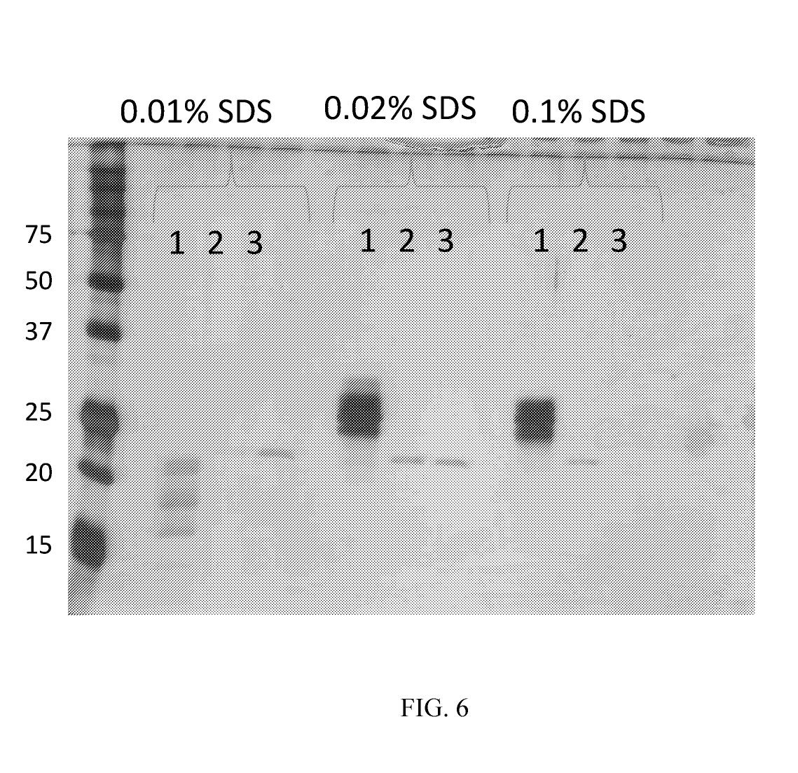

For the optional digestion of intact proteins, proteins can be digested in solution after fraction collection from gel electrophoresis even prior to SDS removal (see Example 5). This can be verified by results shown in FIG. 6 where fractions eluted from gel electrophoresis were digested offline prior to SDS removal. This is accomplished by having no SDS in the elution buffer.

C. Auto-Sampling

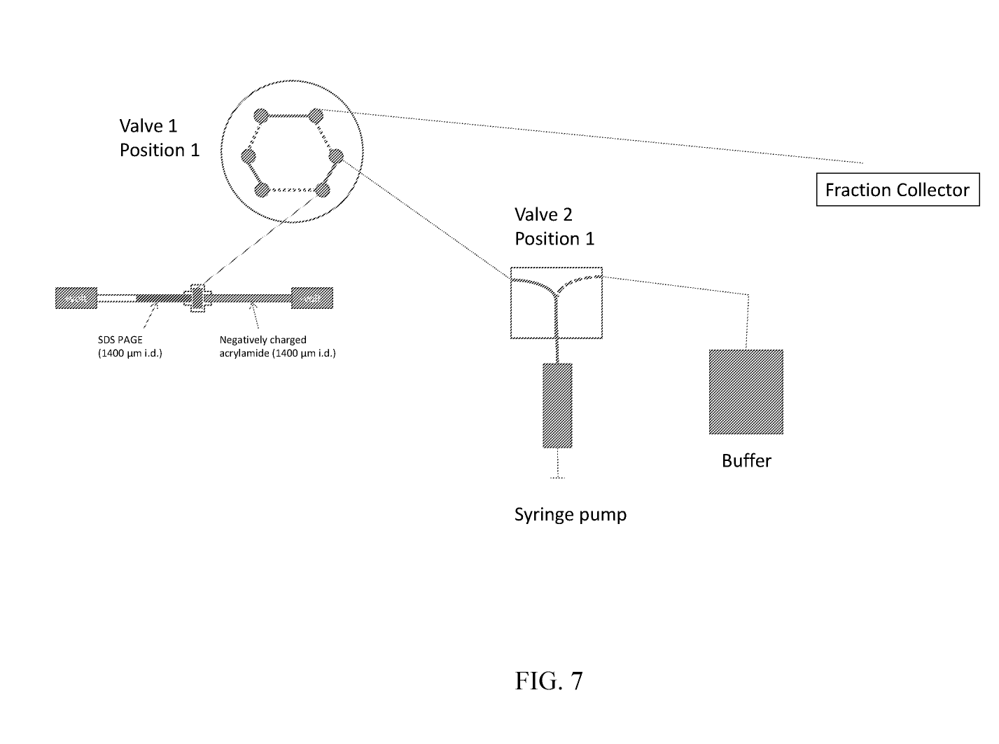

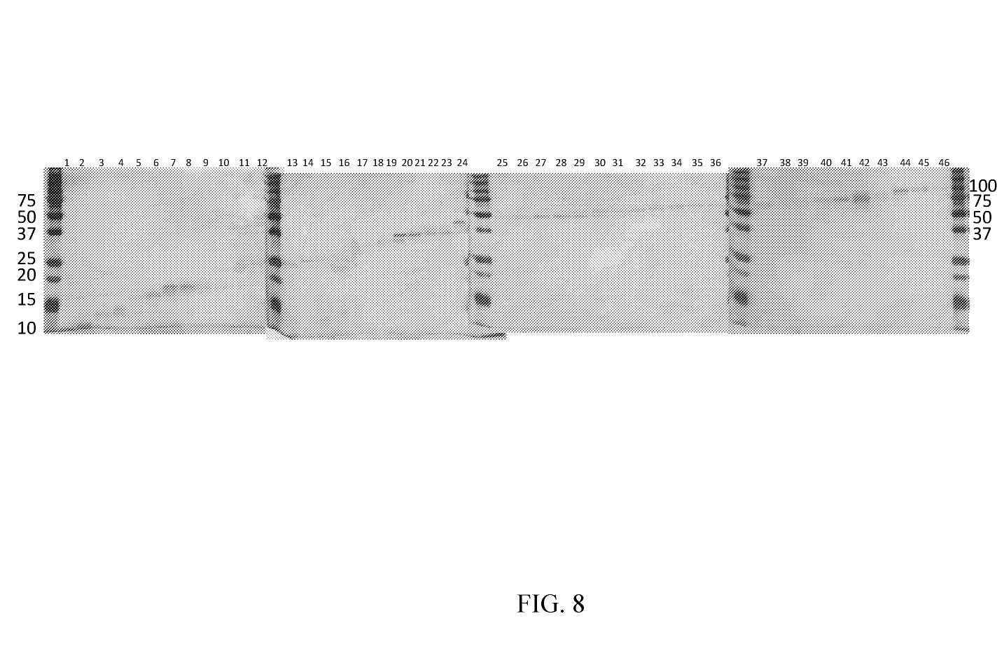

In one embodiment shown in FIG. 7, samples can be effectively transported with a simple auto-sampler with a built-in syringe pump. FIG. 8 shows a gel image of fractions collected from an auto-sampler using a GELFrEE separation over an eight hour electrophoresis run. Example 4 describes one method for auto-sampling.

### Matrix Processing Module

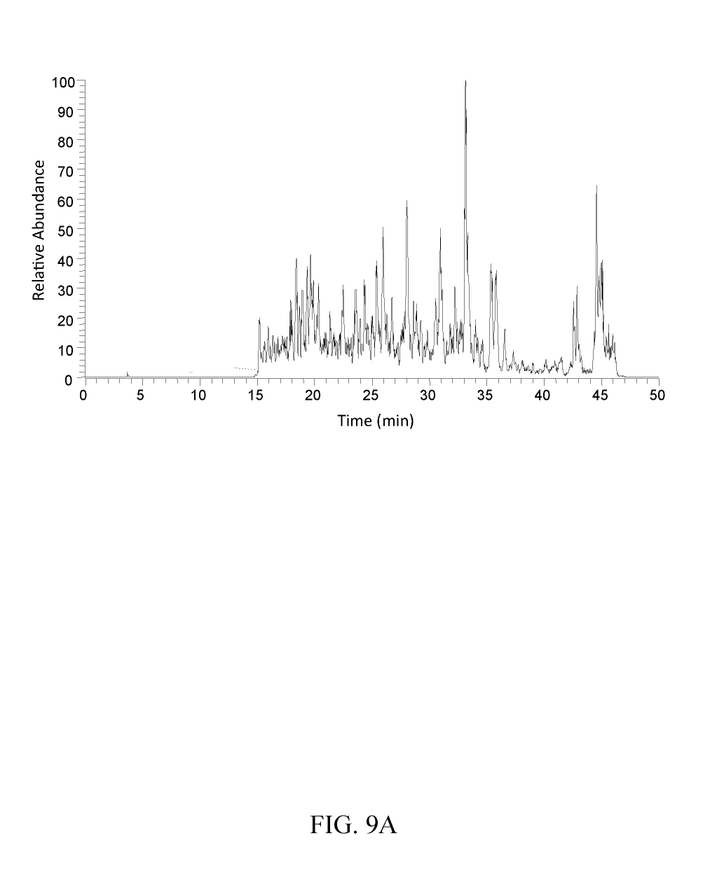

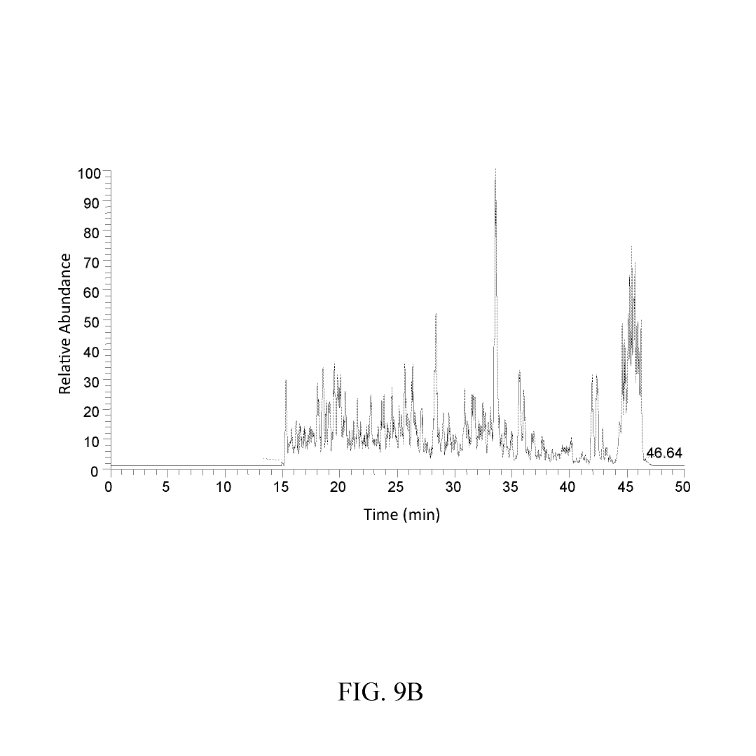

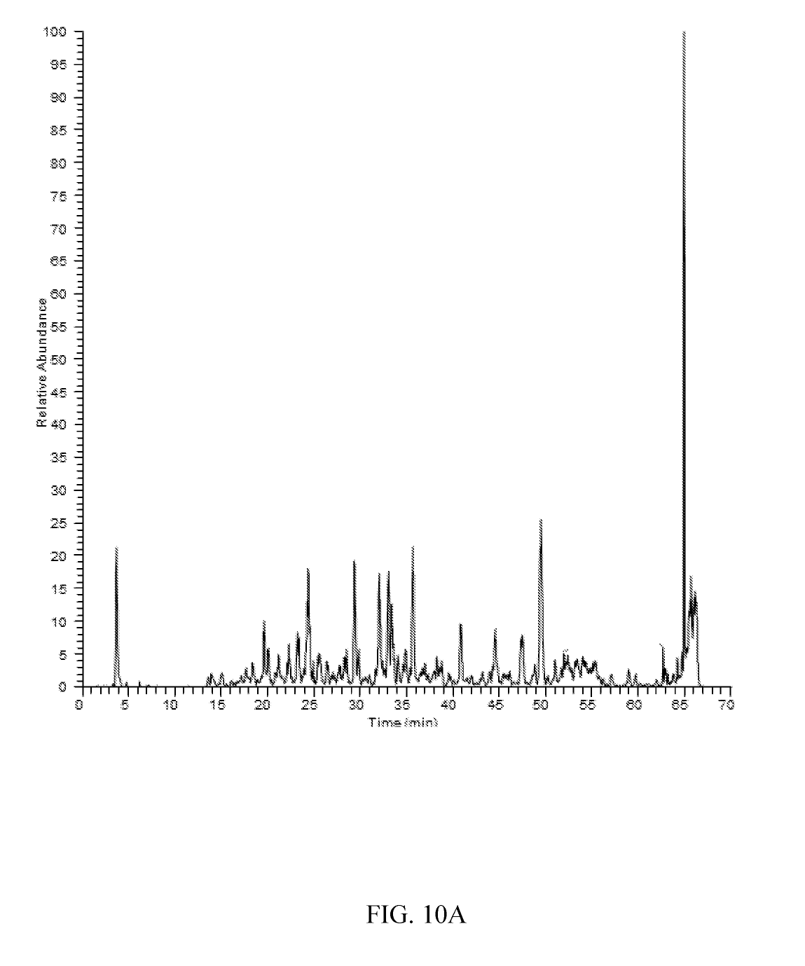

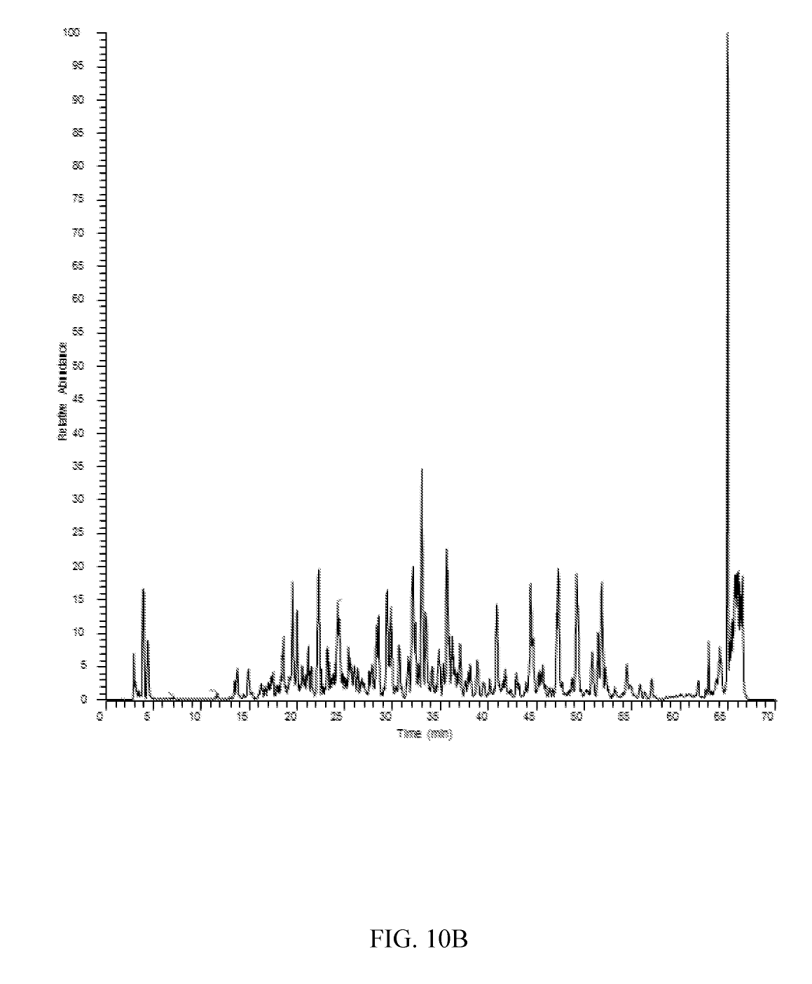

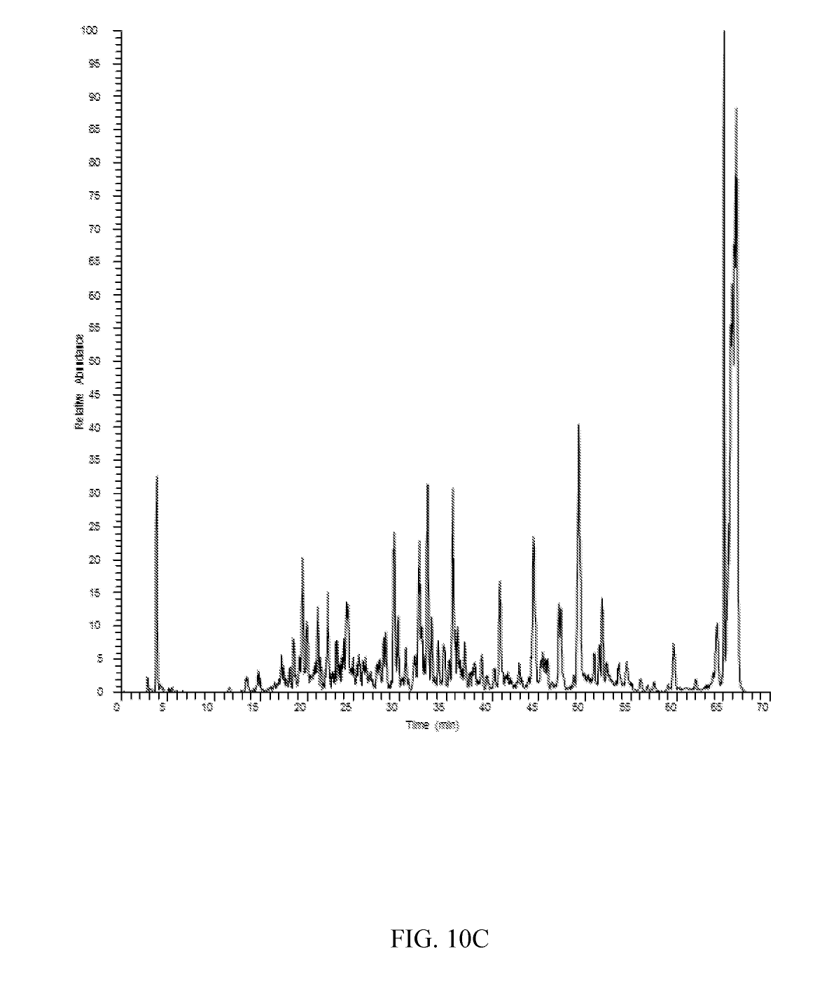

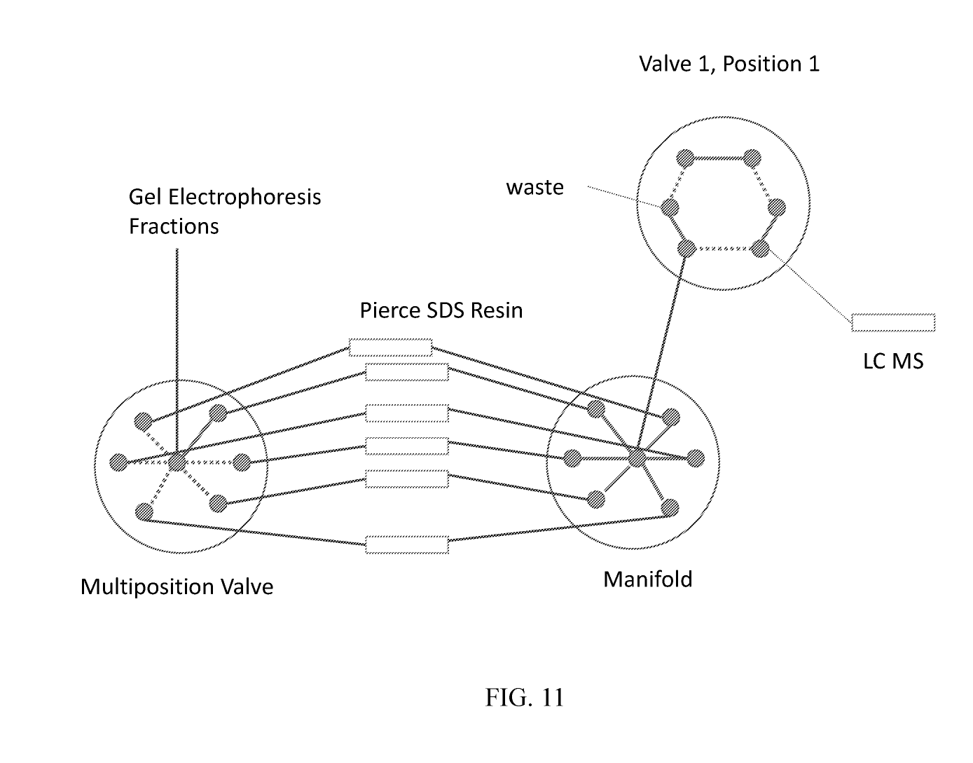

Removal of matrix components used during protein separation, such as detergents like SDS and other MS-interfering agents, can occur either through an AF4 device, a proprietary Pierce SDS removal resin or an ion exchange resin, among other methods (see Example 5). The strategy of removing a matrix material through an AF4 can be found in U.S. Pat. No. 8,298,394. For SDS removal using ion exchange in certain preferred embodiments, weak anion exchange is preferred over cation exchange. In this strategy, proteins are bound to the anion exchange resin, whereas SDS is removed. In the presence of significant amounts of SDS, RPLC resolution and mass spectrometry sensitivity will deteriorate. FIG. 9A,B shows that the RPLC resolution is not affected and the mass spectrometry sensitivity has not decreased when compared to the control suggesting that SDS removal is effective with cation exchange for peptides. FIG. 10 shows that for peptides, little differences in SDS removal efficiency exist between the use of proprietary Pierce SDS resin (FIG. 10B) and the cation exchange resin (FIG. 10C; cf. FIG. 10A for classical protein recovery via precipitation) FIG. 11 provides a schematic of an embodiment of a online matrix processing module comprising a plurality of columns containing Pierce SDS resin for SDS removal followed by RPLC coupled to mass spectrometry.

### Miniaturized AF4 Device for On-Line, High-Throughput Fraction Processing for Matrix Removal

A modified and miniaturized version of an AF4 device is provided that emphasizes speed and efficient removal of matrix components (for example, surfactants, salts, among others) over resolution of protein fractions. Therefore, the modified AF4 device works optimally when the deficiencies of protein separation can be compensated by an additional pre-fractionation step.

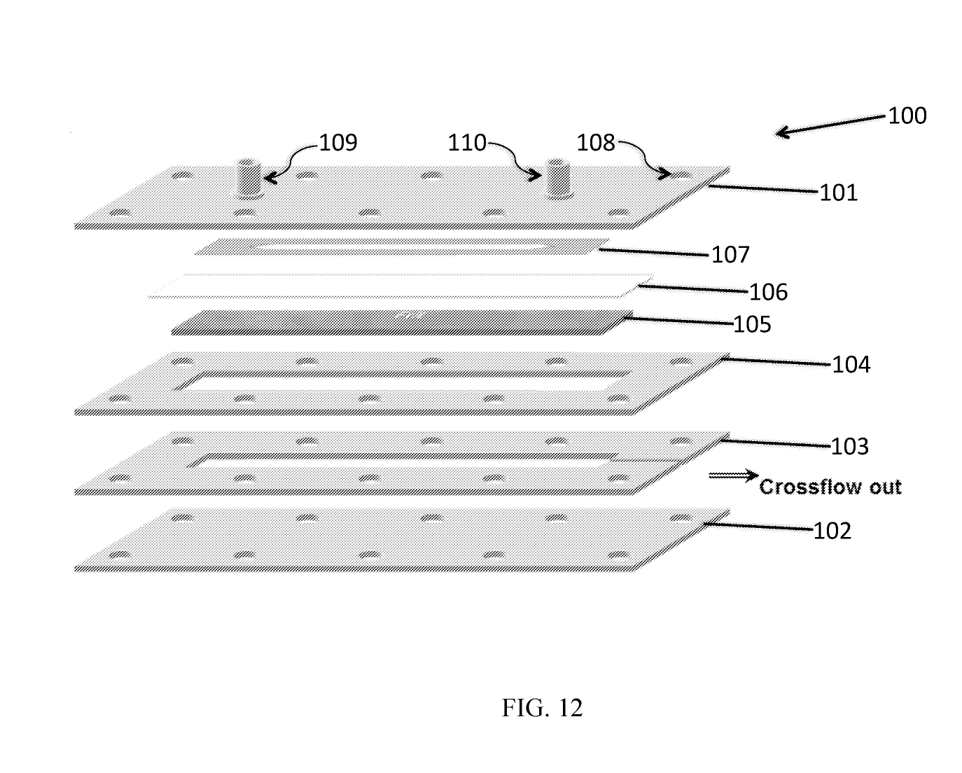

Referring to FIG. 12, the exterior design of a modified AF4 device 100 is similar to chip-type asymmetrical flow field-flow fractionation (AF4) channel. The main body is preferably composed of four stainless steel (SS) plates having each different function. In a preferred embodiments, all of the SS plates are preferably polished surfaces and having preferred exterior dimensions of about 13.0×4.5 cm², though other materials and/or dimensions can also be used. A first plate is a top clamping plate 101; the second plate is a bottom clamping plate 102; the third plate is a reservoir plate 103; and the fourth plate is a frit holder plate 104.

A frit 105 is position in the frit holder plate 104. Frit 105 is composed preferably of sintered stainless steel. Frit 105 has a pore size with dimensions in the range from about 2 μm to about 25 μm, wherein a 10 μm pore size is a preferred pore size.

Reservoir plate 103 has a function of housing the frit and reservoir.

A membrane 106 (preferably regenerated cellulose) is located on frit holder plate 104. The pore size of membrane 106 is dependent on sample protein size; generally, a membrane 106 with a MWCO having a preferred cut-off in the range from 10-20 kDa possesses good recovery and removal efficiency.

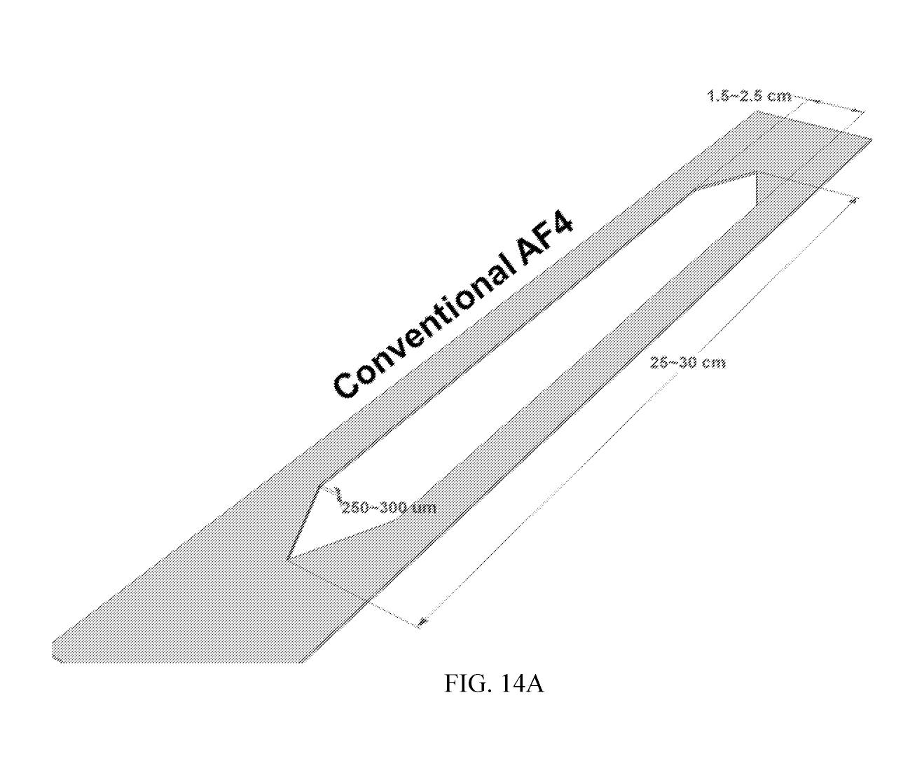

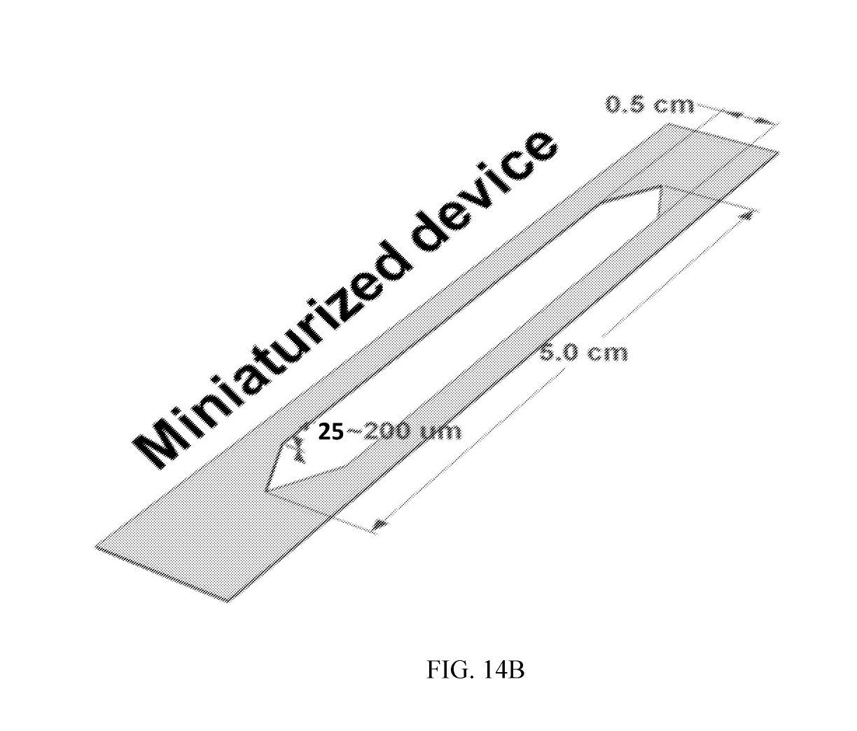

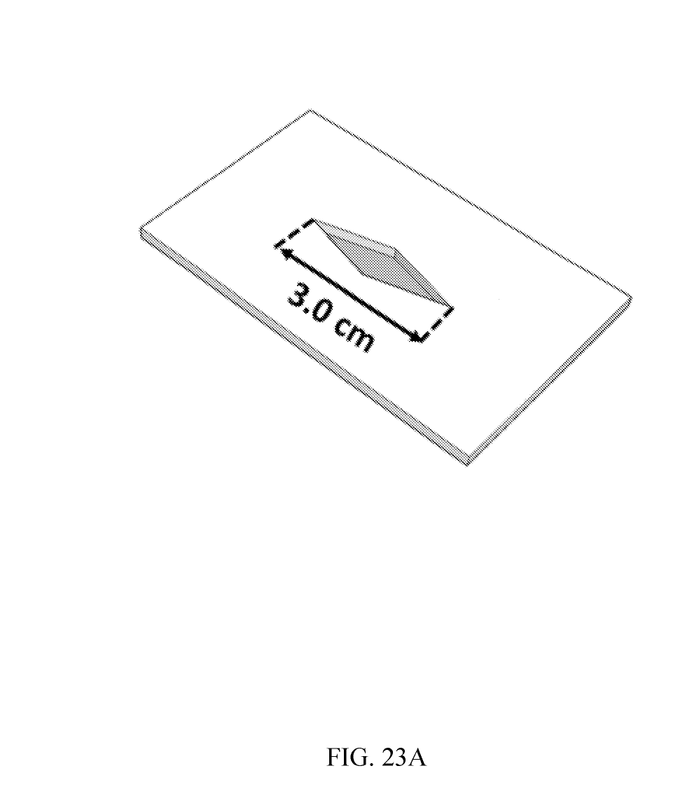

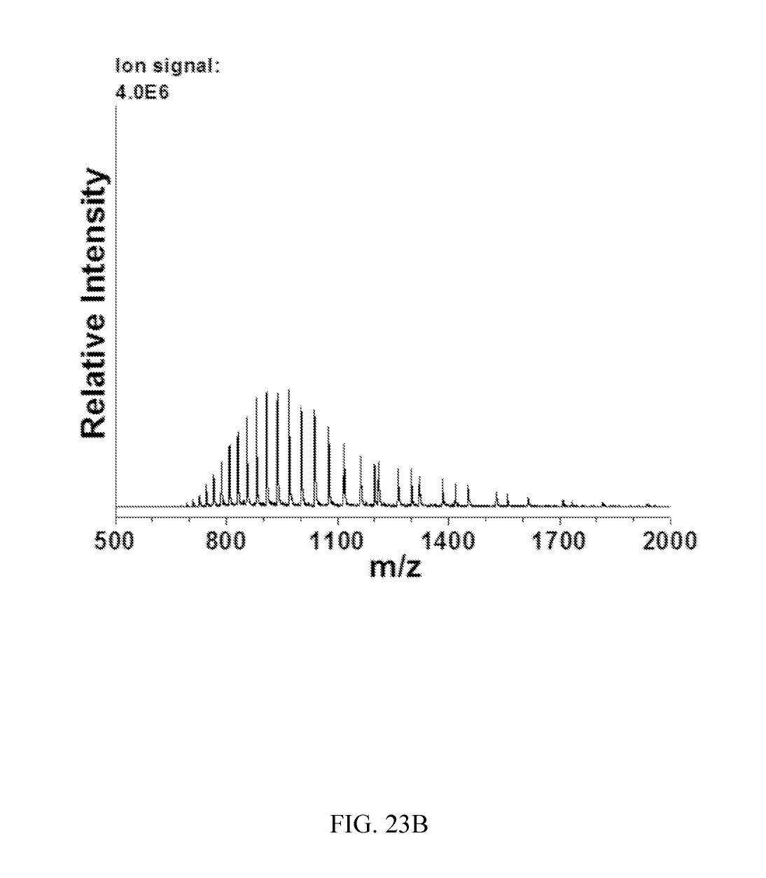

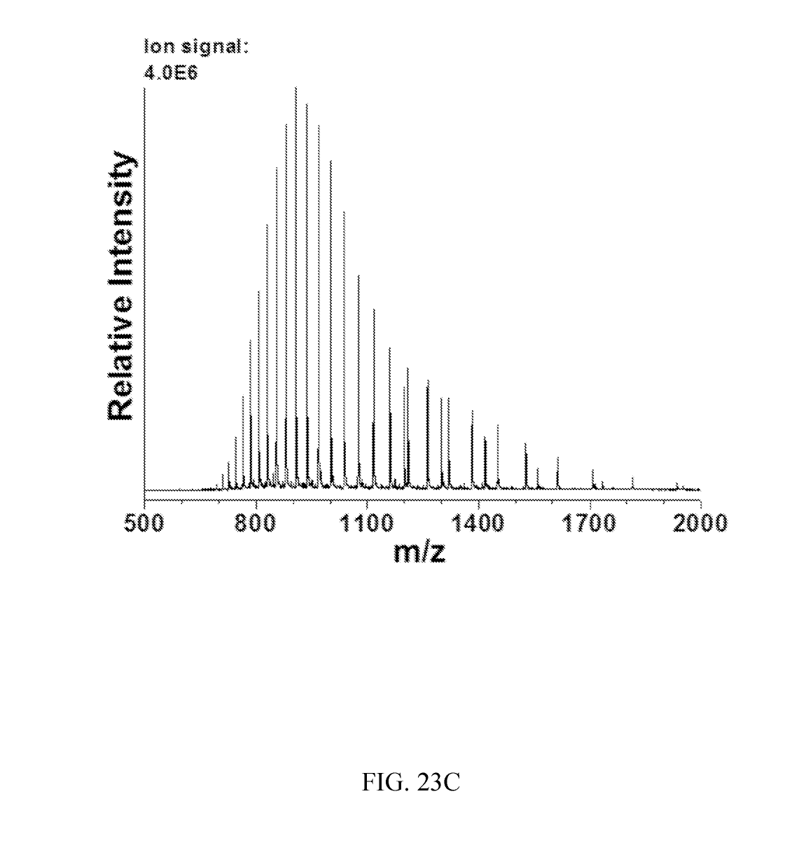

A spacer 107 is coupled to the bottom interior surface of top plate 101. Spacer 107 is preferably composed of a material having chemical compatibility and resistance to degradation, deterioration, or loss when contacted with fluids and solvents used in the system. Spacer 107 is preferably composed of a material having acid resistance to typical MS-based solvent systems. Exemplary materials for spacer 107 include PET and PEEK, among others. Spacer 107 also includes a cut-out. The cut-out of spacer 107 can be of any shape. In preferred embodiments, the cut-out of spacer 107 has a substantially rectangular shape or an elongated hexagonal shape, such as that shown in FIG. 14B (cf. a conventional spacer design in FIG. 14A). In other preferred embodiments the cut-out can be any shape except trapezoidal or exponential shapes (see, e.g., Example 13 and FIG. 23A).

The cut-out of spacer 107 provides a channel space that the carrier solution can flow into and wherein the matrix removal process is performed. The shape of the inner channel space, as defined by the cut-out in spacer 107, is preferably like a stretched hexagon with ribbon shape and is made by cutting a spacer 107 composed of PET film. The thickness of spacer 107 is critical to determine the retention time of proteins, easily predictable by well-established AF4 theory. In previous experiments, a preferred thickness in the range from about 150 μm to about 200 μm is optimal for fast removal and short retention time. The length of inner space (from tip-to-tip length between two inlet ports) can be varied, but preferred lengths in the range from about 5.0 cm to about 7.0 cm shows satisfactory performance attributes (for example, efficient removal and short retention time) for certain preferred embodiments. The schematic diagram of spacer is described in FIG. 14B. These design details, as well as the resultant novel performance attributes, are discussed further below.

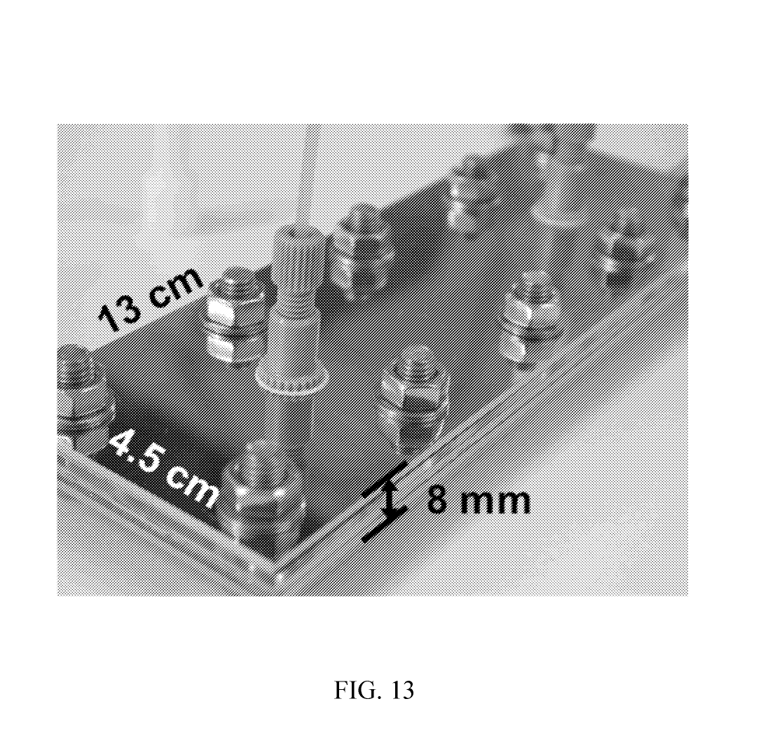

Referring again to FIG. 12, the assembly of plates 101, 102, 103 and 104 is as follows. Plates 102, 103 and 104 are coupled together preferably using silicone rubber. Top plate 101 and the coupled combination of remaining plates 102-103-104 of AF4 device are held together in a clamped arrangement by a plurality of fasteners (for example, bolts and nuts) that pass through a plurality of holes 108 disposed throughout, though preferably on the perimeter of, plates 101, 102, 103 and 104. For the connection of pump and mass spectrometry, two holes are drilled (for example, 1/16″ diameter) into top plate 101 for the flow inlet 109 from pump and outlet 110 to mass spectrometry, and the tubing connections are made through two inlet port assemblies (exemplary assemblies were from Upchurch Scientific) that are attached thermally on top plate 101. Teflon tubings can be connected by insertion through top plate 101 such that the tube end can be extended to the other surfaces of the SS plates. The schematic diagram of the modified AF4 device is shown in FIG. 12 and photograph is illustrated in FIG. 13.

### Critical Innovation Parameters to Modified AF4 Channel Design: Improvement by Miniaturization

The successful coupling to mass spectrometry can be achieved with reducing flow rate with miniaturization of the AF4 device 100. The inner space of miniaturized device as compared against a typical prior art AF4 device, can be defined by the cut-out of spacer 107, as shown in FIG. 14A and 14B.

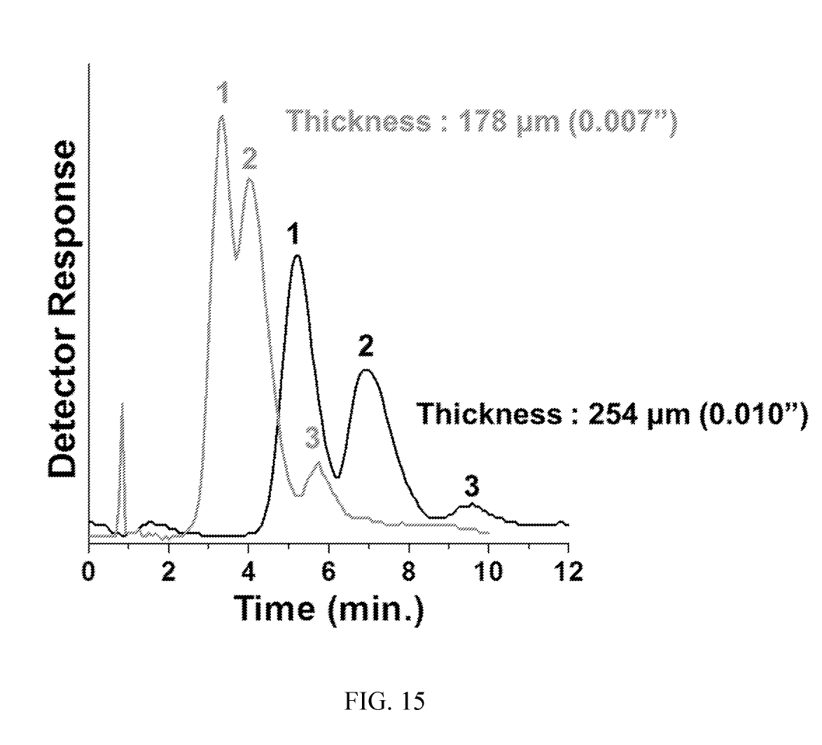

An approximate retention time of an AF4 device channel can be expressed Eq. 1.

$${t}_{r} ≅ \frac{πη {w}^{2} d}{2 kT} ln ( \frac{{\dot{V}}_{O}}{{\dot{V}}_{L}} ) \qquad ( Eq .1 . )$$

wherein w is a channel thickness of AF4 channel and \acute{V}_{O}, \hat{V}_{L} is an initial flow rate introducing the AF4 channel and a flow rate at the endpoint of AF4 channel, respectively. The other terms are related to diffusion coefficient of sample particles. Therefore, the optimization of channel thickness is required for efficient separation of various samples and 250˜300 μm of thickness is used to separate proteins in many applications (FIG. 14A). However, using very slow flow rates for electrospray ionization can cause long retention time and increases analysis time. Therefore, miniaturization of the AF4 channel by decreasing channel thickness is desired to minimize retention time. Referring to FIG. 14B, in this approach, a channel thickness can be adjusted to 150-200 μm and can reduce the retention time by 50% as can be determined through Eq. 1. In the previous data described in FIG. 15, reducing the channel thickness from 254 μm to 178 μm (from 0.010 inch to 0.007 inch) significantly reduced the retention time on protein standards. In the case of carbonic anhydrase and BSA, the retention times were reduced by 46% and 50% respectively. These values fall in accordance with the AF4 theory. However, it should be noted that thinner channels provide relatively lower separation efficiency or resolution. The resolution of separation was reduced by 23.5% as predicted (from 1.38 to 1.06, calculated from theory in chromatography and void time was excluded). This particular modification of the AF4 device is counter-intuitive and reflects an inappropriate design choice where the intended application of the AF4 device is for achieving high separation efficiency and fraction resolution. Yet since SDS removal using the modified AF4 device of this disclosure is preferably coupled to a prefractionation step, such as the GELFrEE separation (see above), having this resolution reduction will not lead to concerns in analytical performance.

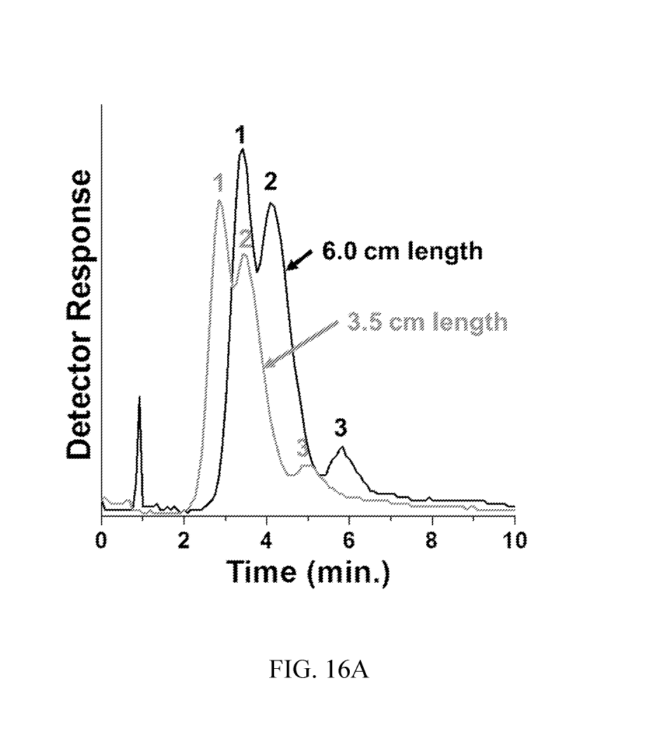

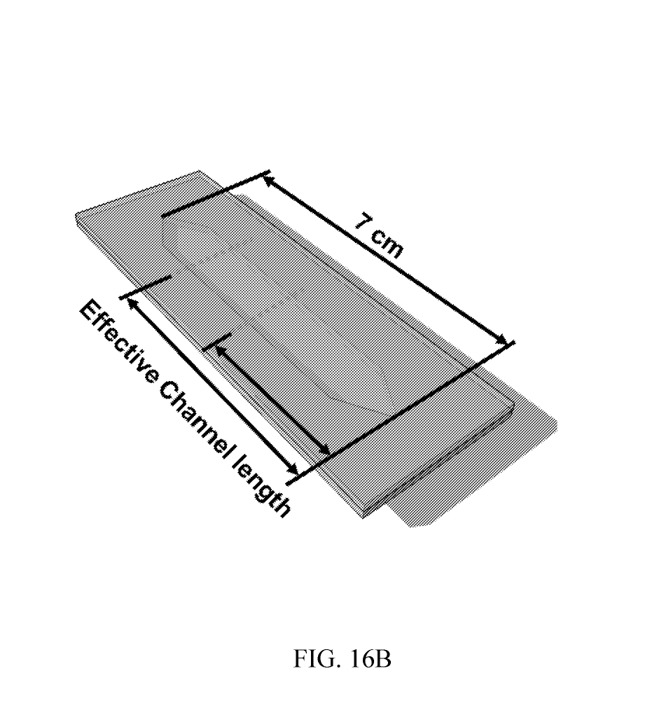

The length of the inner space of the AF4 device 100 is related to the resolution in separation analogous to that observed in chromatography. Typical prior art AF4 channels are optimized at 27 cm length for various types of analytes, but short channels below 10 cm can be sufficient for protein separation. Moreover, the extremely short channel is sufficient for matrix removal in a protein sample. The effect of channel length is illustrated in FIG. 16. The effective channel length (from the focusing/relaxation position to the port for outflow) was decreased from 6.0 cm to 3.5 cm by changing the length or inner diameter of the connection tubing (FIG. 16B). As a result, the retention time of carbonic anhydrase and BSA was reduced by 23.2 and 21.2%, respectively (FIG. 16A). The width of each peak is similar to the result from a 6 cm-channel so the plate number was decreased proportionally to channel length. However, as mentioned, high performance separation resolution is secondary to speed of surfactant removal because an additional separation device can be adopted prior to the AF4 separation.

The miniaturization of the modified AF4 channel is desirable to enable direct coupling with ESI mass spectrometry to allow slower flow rates from the modified AF4 for stable electrospray ionization. The dimension of a prior art AF4 channel is approximately 1.5 cm×27.0 cm×275 μm (width×length×thickness) and 1.0-10.0 mL/min of initial flow rates (\acute{V}_{O}) and 0.1-1.0 mL/min of outflow rates (\acute{V}_{L}) are used. This dimension is suitable for 1) observing a sample size distribution using a general HPLC detector or 2) fractionating a sample mixture for further analysis. However, with these dimensions, using slow outflow rates at nanoliter or microliter per minute scales is not recommend due to excessive increases in retention times. The increasing retention times can be an obstacle for mass spectrometric analysis because of excessive sample dilution. For example, when a 1 μL protein sample at 1 mg/mL is injected into a prior art AF4 channel at low flow rates, the concentration of eluted fraction can be decreased 200-1000 times (0.01-0.001 mg/mL). The low protein concentration must be enriched in order to be detected, which will require an additional concentration step.

In sharp contrast, use of the miniaturized device results in a dilution factor of only 10-20 for the applied protein sample, rendering the device suitable for processing proteins for direct analysis without a concentration procedure. Moreover, a miniaturized AF4 device can be situated closer to the ionization source of a mass spectrometer, thereby minimizing dead volume and enabling reduced delivery time and sample dilution. The miniaturization of the inner space of the modified AF4 device described in FIG. 14B can reduce the required flow rates (\acute{V}_{O},) for the separation of proteins. The channel area is about 1/10 the size of prior art designs, so migration of proteins can be achieved with relatively low initial flow rate (\acute{V}_{O}, 0.05˜0.1 mL/min) and very low outflow rate (\acute{V}_{L}, 0.3˜10.0 μL/min), which is suitable for analysis with mass spectrometry.

In certain embodiments, the length of inner space (that is, the tip-to-tip length between an inlet port and an outlet port) can be varied (see, e.g., Example 13), but 5.0-10.0 cm of length can provide satisfactory results in separation. Spacer cutouts having lengths less than 5.0 cm can adequately separate protein from small molecules like surfactants and still provide rapid delivery of protein for mass spectrometry analysis. The current commercial nanoport as inlet port fitting (IDEX CORP. (Lake Forest, Ill. (USA))) has a diameter of 8.4 mm in the exterior design; therefore, a spacer having a 3.0 cm length cutout was designed to provide sufficient space for installing three inlet ports in a row. The thickness of the spacer is dependent on the available material. For example, PEEK sheets (or PEEK tapes) having a thickness of 0.005″ (127 um) or 0.010″ (254 um) are available from commercial sources. A PEEK sheet or tape having a 0.005″ (127 um) thickness film is preferred because spacers having this thickness can permit fast elution of proteins. In other embodiments, thinner PEEK tape having an adhesive backing can provide a smaller thickness dimension, such as about 10-12 microns. The diamond shape of spacer, as viewed from the top perspective (that is, along the length-width dimension), was designed for efficient removal of small molecules. The tapered shapes at each end of the spacer are designed to expand solvent flow from the small inlet port to the maximal breadth of the spacer as well as refocus flow to a small area prior to extraction from the outlet port. The maximum width at the center of spacer is preferably narrower than the width of the frit (1.5 cm) so that the carrier solution permeates the frit to the crossflow outlet. FIG. 23A illustrates an exemplary design of a spacer having a cutout with a diamond shape of 1.0 cm width and a 3.0 cm length.

The preferred dimensions of the inner channel space for the modified AF4 device 100 (and 200, see below) have a length in the range from about 3.0 cm to about 10.0 cm, including about 0.05 cm incremental variations within that range (for example, about 3.05 cm, 3.55 cm, 4.75 cm 5.85 cm, 7.25 cm, 8.50 cm and 9.95 cm, among others); a width in the range from about 0.20 cm to about 1.00 cm, including about 0.05 cm incremental variations within that range (for example, about 0.25 cm, 0.50 cm and 0.75 cm, among others); and a height (thickness) in the range from about 10 microns to about 300 microns, including incremental variations within that range (for example, about 12 microns, 15 microns, 20 microns, 25 microns, 50 microns, 78 microns, 100 microns, 155 microns, 200 microns and 290 microns, among others). A highly preferred length, regardless of shape of the spacer cutout, falls in the range from about 3.0 cm to about 6.00 cm, including 0.05 cm incremental variations within that range, such as 3.05 cm, 3.75 cm, 4.55 cm and 5.60 cm, among others. A highly preferred thickness, regardless of shape of the spacer cutout, falls within the range from about 100 um to about 150 um, including integer incremental variations thereof, such as 125 um, 127 um and 130 um, among others. Using a rhombus as a reference spacer cutout shape (such as the diamond shape depicted in FIG. 23A), AF4 channels described herein can have spacer cutouts with an approximate surface area in the range from about 0.30 cm² to about 5.00 cm² and an approximate volume in the range from about 3×10⁻⁶ cm³ to about 1.5×10⁻³ cm³. One having ordinary skill in the art can appreciate that the surface area and volume of the spacer cutout will depend upon the overall shape of the spacer cutout, based upon consideration of geometric principles.

### Mass Spectrometer Module

The mass spectrometer module can comprise any mass spectrometer configuration amenable for protein mass spectrometry analysis by either the bottom up or top down approaches. Exemplary mass spectrometer configurations for this purpose may use a variety of ionization sources such as SLD, MALDI, ESI, APPI, APCI, among others and a variety of mass spectrometric detectors such as ICR, QLT, Orbitrap, TOF, Sector, QMF and others.

### On-Line Connections Between Modules of the System

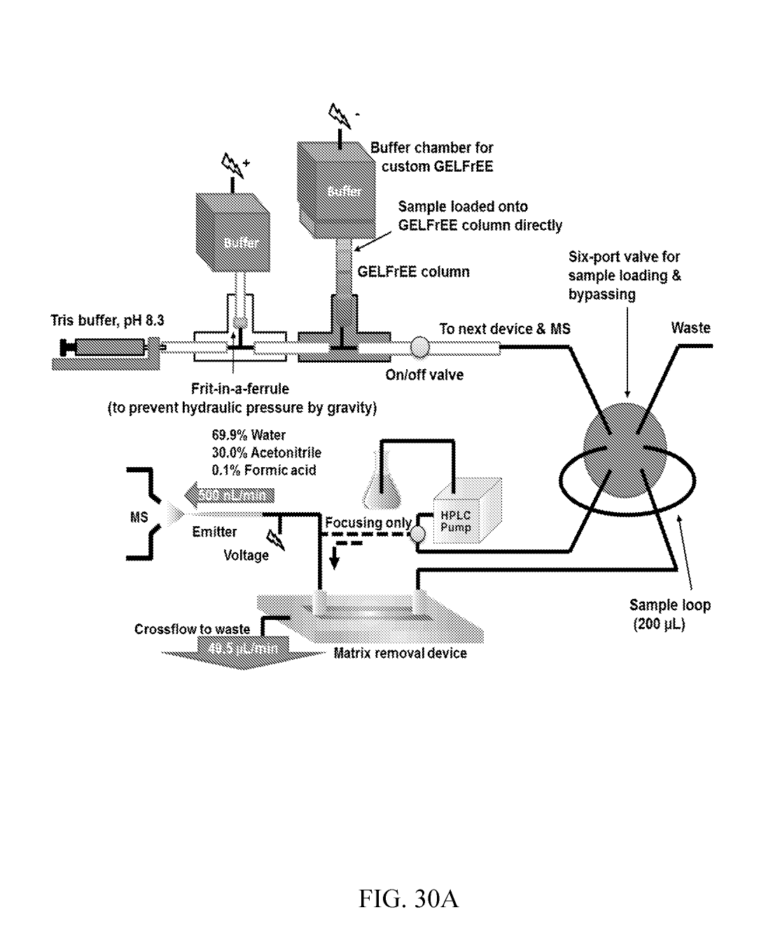

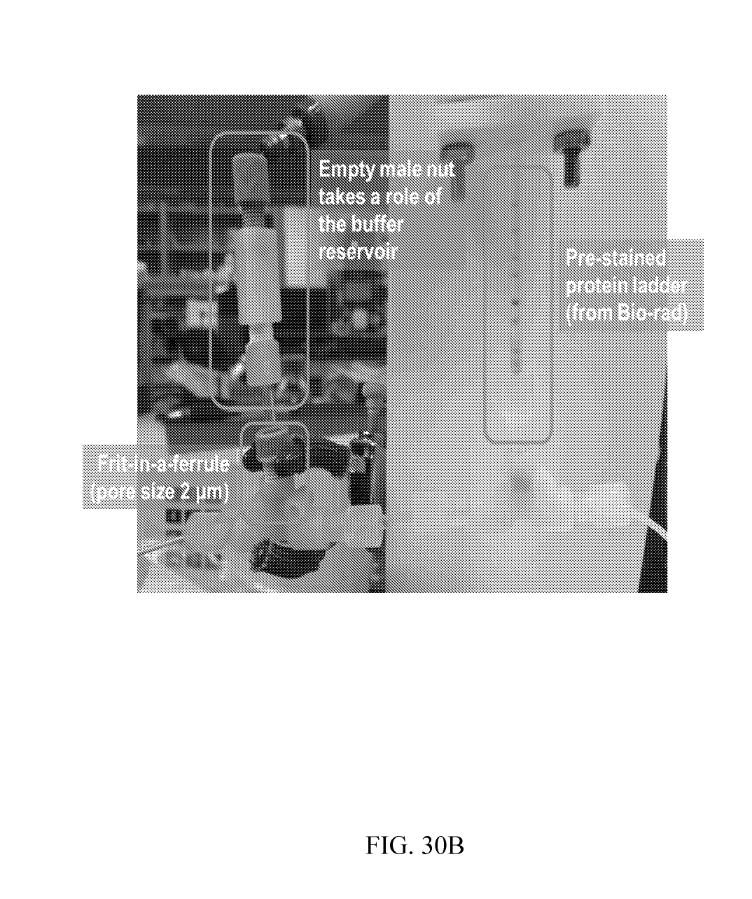

The connection amongst the removal device, separation equipment and mass spectrometry is achieved preferably with Teflon and fused silica capillary tubing. The Teflon tubing ( 1/16″ O.D., 0.010˜0.030″ I.D.) can be utilized in the connection between a pump (or alternative separation device) and a removal platform for transferring large volume of carrier solution. Narrower fused silica capillary (360 μm O.D., 30˜150 μm ID.) tubing can be used to couple to mass spectrometry to avoid delays. The schematic diagram of setup (removal device coupling to mass spectrometry) is described in FIGS. 3 and 30 (see, e.g., Example 17).

### Subsystem Designs and Capabilities

The present system can be configured in any combination of subsystems. A preferred subsystem is a protein separation module coupled to matrix processing module. A highly preferred subsystem is a protein separation module comprising an electrophoresis system that includes a capillary gel column for separation of protein fractions, a collection chamber in a tee configuration and a polyacrylamide gel retention, as illustrated, for example, in FIG. 4 of this disclosure. A highly preferred matrix processing module comprises the modified AF4 device 100 and 200 as disclosed, for example, in FIGS. 12-14 and 26 and in Example 15 of this disclosure. Such subsystem combinations enable any MS instrument to be retrofitted for use to process high-throughput proteome samples from clinical specimens (see, e.g., Example 16).

### Advantages

1) Fast, Simple Removal of Surfactant

This technique is able to rapidly remove surfactant such as sodium dodecyl sulfate (SDS) or coomassie brilliant blue. Present technology, based on centrifugal filter device, require over an hour to achieve concentration high enough to be analyzed with mass spectrometry. Alternatively, using dialysis requires over 24 hours for purifying sample. Moreover, the use of common precipitation procedures for surfactant removal is tedious, time consuming, results in low recoveries and disintegrates native protein structures.

In sharp contrast, surfactant removal with this platform requires less than 10 minutes for complete removal. Additionally, the ability to automate the sample collection and transfer to mass spectrometry further reduces labor and expands the technology to non-expert users. Additional advantages of the platforms provided herein are amplified by sensitive methods for achieving surfactant removal, such as modifying flow rates and ionic strength of the carrier medium. These advantages are described in greater detail in Examples 6-12.

2) Simultaneous Buffer Exchange and Concentration

The volatile buffer condition is essential in electrospray ionization, so buffer exchange is highly required in many protein samples. The new platform can completely exchange the buffer condition with a cross flow action. Additionally, the elution volume from the device is irrelevant to an initial sample volume so concentration efficiency can be increased as the initial sample has low concentration.

3) High Recovery of Protein

Popular precipitation methods to remove SDS using organic solvent provide low recovery of proteins. The commercialized technique using a spin-column results in undesirable binding of proteins to column resin. Due to its mild conditions, filtration is the most favorable method to removal matrix from protein sample.

This invention is a new flow-based method that operates similarly to filtration with the additional advantage of very rapid sample cleanup. By implementing embodiments having superior leakage-free operation, improved spacer design, and chemically resistant spacer material compositions, further improvements in protein recovery, in protein purity devoid of contaminants (i.e., from dissolved spacer materials reacting with carrier medium), in MS signal to noise ratio (i.e., increased sensitivity). These features are further detailed in Examples 13-15.

4) Economical and Easily Maintained

In general, a centrifugal filter device or a spin column is disposable and uneconomical (currently ˜$7 for each sample of 100 μL). In sharp contrast, in this invention, most parts are reusable where only the replacement of membrane is required (˜$0.1 for each sample of 100 μL). The main parts of the device are only composed of a few stainless steel plate, sintered filter and membrane (less than currently $100 for making a prototype). Additional equipment and accessories such as valve, tubing and pump are typical items used in conventional chromatography.

5) Automated System

All process of removal can be controlled by automated flow from pump and valve action that reduces human error. In addition, when a prefractionation device is attached prior to the new platform, the early stage (sample injection, separation, fractionation, preparation and analysis) can readily be adapted to be performed as one system.

### EXAMPLES

The disclosure will be more fully understood upon consideration of the following non-limiting examples, which are offered for purposes of illustration, not limitation.

The methods below describe the use of the disclosed apparatus system and subsystems for either analysis of intact proteins or peptides using mass spectrometry. Here, the separation can occur through a variety of size based separations including SDS capillary gel electrophoresis, GELFrEE, RADGEL and preparative gel electrophoresis. Fraction collection is performed automatically with an autosampler. For Bottom Up applications, the invention describes a strategy for online immobilized tryptic digestion, SDS removal with anion exchange and finally LC-MS. The method has a preference for digestion prior to SDS removal to ensure high recovery. Alternatively, for intact protein analysis, the SDS removal can occur through a variety of strategy such as size exclusion, precipitation and ion exchange. The disclosure describes a methodology for SDS removal using the miniaturized AF4 device technology.

### Example 1

Materials

Milli-Q grade water was purified to 18.2 mΩ/cm. All reagents for gel electrophoresis were obtained from Bio-Rad. 3.5 kDa molecular weight cut-off dialysis membranes were purchased from ThermoFisher Scientific.

### Example 2

Sample Preparation

Lyophilized cells of H1299 cells were resuspended with 5×Laemmli gel loading buffer (0.25 M Tris-HCl pH 6.8, 10% w/v SDS, 50% glycerol, 0.5% w/v bromophenol blue). Samples were heated to 95° C. for 5 minutes prior to loading onto the gel electrophoresis setup.

### Example 3

Gel Electrophoresis Separation

Separation can occur using a variety of continuous elution gel electrophoresis separation such as GELFrEE, SDS capillary gel electrophoresis or the RADGEL. The following will describe the separation process when using RADGEL. During sample loading, the casting chamber was replaced with a loading chamber. Operation of the device is described in three distinct stages: (1) sample loading, (2) separation and (3) auto-sampling collection. For sample loading, the electrolyte chambers of the device and collection chamber, were completely filled with running buffer (0.192 M glycine, 0.025 M Tris, 0.1% SDS (Laemmli, 1970)) with the auto-sampler as described in Example 4. Separation occurred with constant application of 240 V across the system. After the sample had entirely migrated into the gel, the loading chamber was replaced with the running chamber. The electrolyte was filled in the chamber and the voltage was reapplied. Collection began when the dye front had visibly entered the collection chamber. During collection, the power supply was paused, and, using a pipette, the entire volume of the collection chamber was transferred to a clean vial. A fresh 1 mL portion of running buffer was loaded into the collection chamber, and the power source was switched on to resume separation. This process was repeated over the course of separation, collecting fractions during each stop-and-go cycle.

### Example 4

Auto-Sampling

Sample collection will occur automatically with a programmed auto-sampler setup shown in FIG. 11. During fraction collection, both valves 1 and 2 are switched to position 1. The syringe pump will then draw the fractions into the syringe. Valve 1 is switched to position 2, and the fractions are collected in a fraction collector. To replenish the collection chamber, valve 2 is switched to position 2 to pick up running buffer. After picking up the sufficient volume of buffer, both valves 1 and 2 are switched back to position 1 and the buffer is replenished for the next run.

### Example 5

Digesting in Solution and SDS Removal for Bottom Up MS Applications

Adjust switching valve to remove the ion exchange column out of line from pump. Wash and activate the immobilized trypsin resin with 10 column volumes of 95 percent digestion buffer (50 mM Tris, pH 8+5 percent acetonitrile). Switch valve to put trypsin resin offline and place the ion exchange column inline and equilibrate with formic acid (pH 2.5). Place trypsin resin and the weak anion exchange column inline. Inject the fractions collected in experiment described in Example 3. Starting with a flow rate of 3 μl/min, wash the protein through the trypsin resin with at least 3 column volumes of the digestion/acetonitrile buffer. Take the trypsin resin out of line. Elute the peptides from the ion exchange column onto the RPLC-MS with appropriate gradient conditions at 300 nL/min. Take the ion exchange column out of line. Place the trypsin resin inline and wash with 10 column volumes of 1:1 digestion buffer:acetonitrile at 3 to 5 μl/min. Repeat the steps for additional fractions.

### Example 6

SDS Removal of Intact Proteins for Top Down MS Applications

This example provides a strategy for using Pierce SDS resin for SDS removal followed by RPLC coupled to mass spectrometry as shown in FIG. 11. The SDS removal columns (packed with Pierce proprietary resin) are first equilibrated with buffer (50 mM Tris pH 7) through the pump. This can occur sequentially or in parallel (setup not shown). The multiple fractions collected as described in Example 4 are introduced into the multiple columns by a pump sequentially. After incubation (2 minutes or less), the eluent containing proteins are pumped to the RPLC trap column by switching valve 1 to position 2. The SDS remains trapped in the resin. The trap column is then connected online to the analytical column after sufficient loading and washing time through a conventional vented tee setup (not shown). The eluent from the RPLC can then be directly introduced into nanospray mass spectrometry. Alternatively, fractions can bypass RPLC and be directly introduced into nanospray mass spectrometry after SDS removal if supplemental electrospray buffer is introduced immediately before nanospray as shown in FIG. 3.

### Example 7

SDS Removal from Samples Using the Modified AF4 Device.

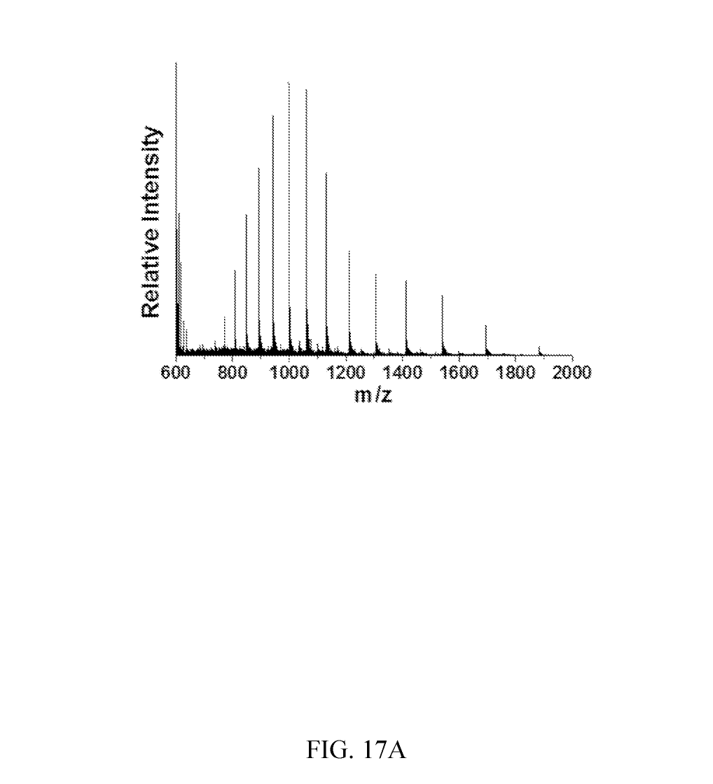

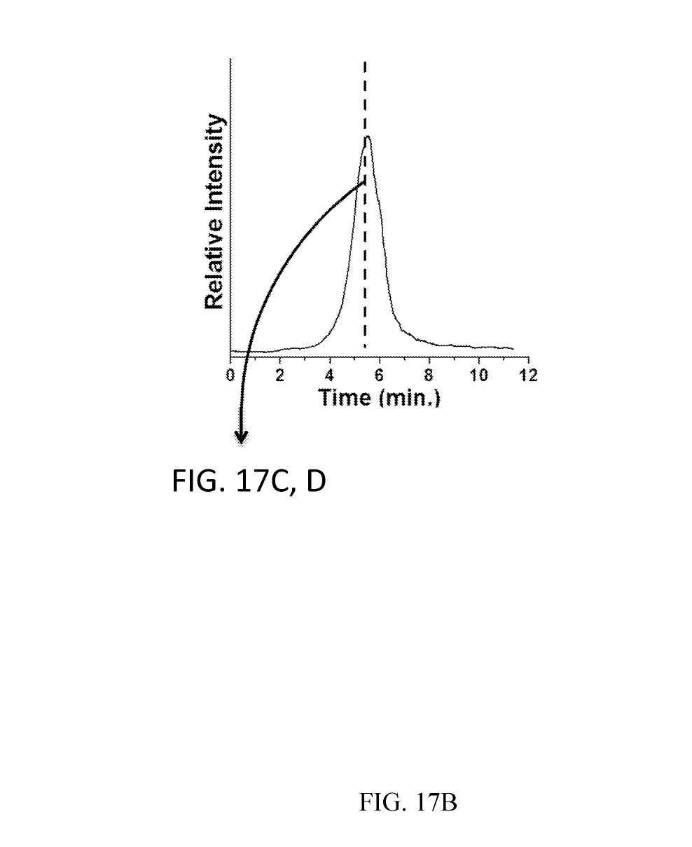

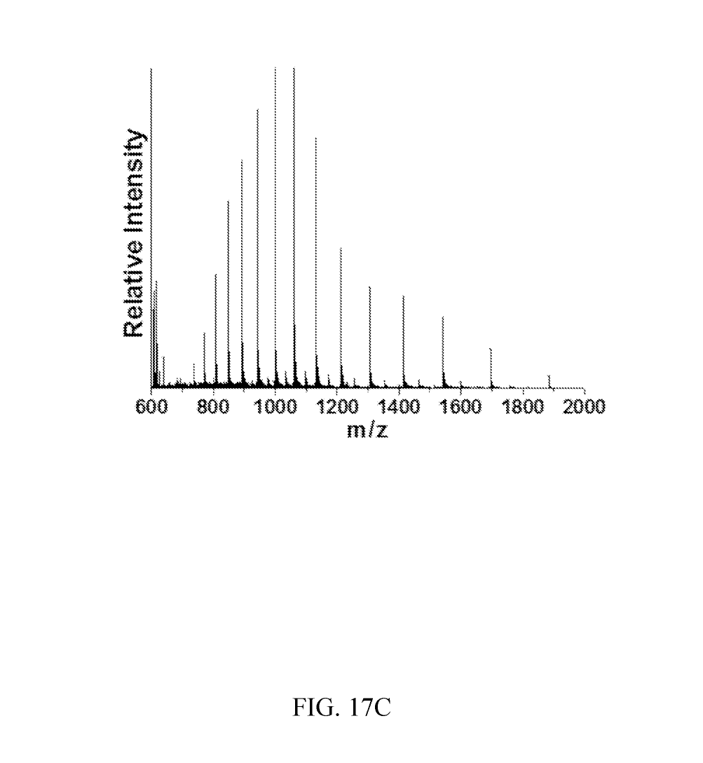

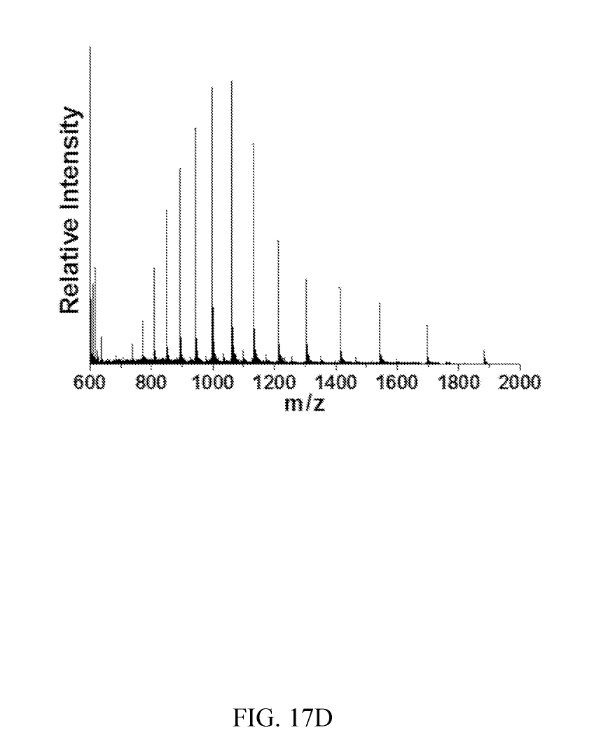

The performance of surfactant removal with the miniaturized AF4 device was demonstrated by mass spectrometry. Myoglobin at various SDS concentrations (0%, 0.1%, and 0.6%) was introduced into the device followed by online ESI-MS (LTQ Velos ion trap from Thermo company). The carrier solution (10 mM ammonium bicarbonate) was used for electrospray ionization. FIG. 17C,D shows the mass spectra of 2.0 μg of myoglobin obtained after SDS (at indicated initial concentrations) was removed using the device. As shown in FIG. 17B, the total ion chromatogram (TIC) showed a peak of myoglobin with a retention time less than 10 minutes. By comparing to the mass spectra obtained with myoglobin in the absence of SDS (FIG. 17A), the signal quality has not deteriorated after SDS removal with removal device was performed (compare FIGS. 17A against 17C and 17D)).

### Example 8

Triton X-100 Removal from Samples Using The Modified AF4 Device.

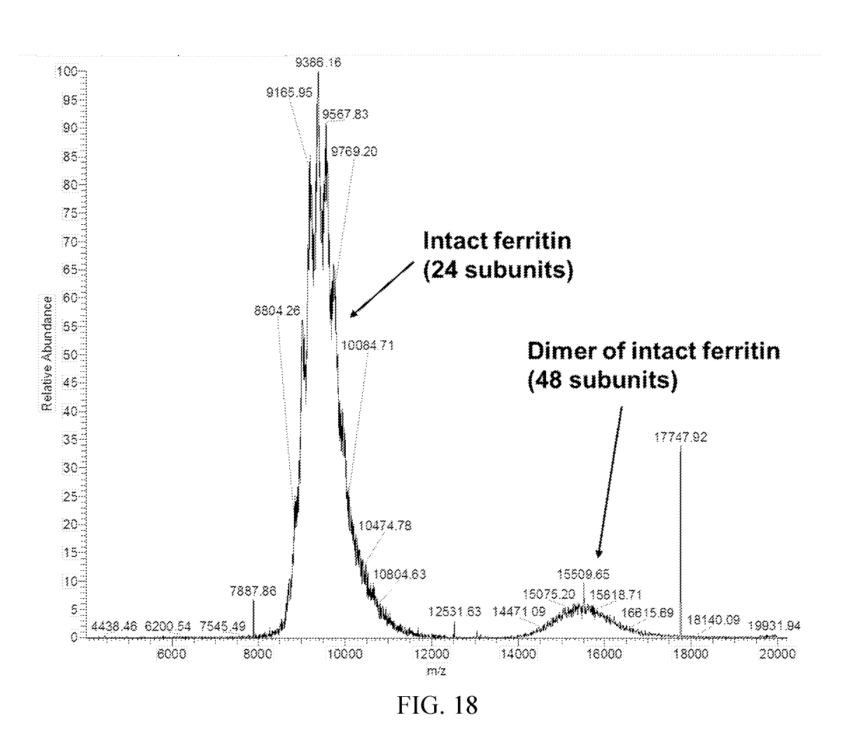

The cleanup of Triton X-100 surfactant from a protein fraction was demonstrated for the recovery of a protein complex. The protein fraction was a holo-ferritin (a complex of 24 subunits) in bis-Tris buffer with 1.0% triton X-100 and it was obtained by electroelution from native PAGE gel bands. For stable electrospray ionization, 0.1 M ammonium acetate (pH 7.0) was used as the carrier solution of the removal device. The removal step required 15 minutes to ensure that minimal interference was observed in mass spectrometry and the fractions were collected at 2 minutes as the retention time. The mass spectrum shows several peaks of the intact state of ferritin complex ions and no peaks were observed from the degraded subunits (FIG. 18). Moreover, the dimer of ferritin (˜960 kDa) could be observed and this spectrum could not be obtained from a typical ferritin solution. These results show that the miniaturized AF4 device can adequately remove triton X-100 detergent from PAGE fractions without degrading protein structure. Retaining the native structure of proteins is significant since these structures are often correlated with biological relevance. However, with other harsh techniques, protein complexes generally disintegrate prior to detection leading to a disconnection in molecular information.

### Example 9

Effect of Retention Time on SDS Removal from Samples Using the Modified AF4 Device.

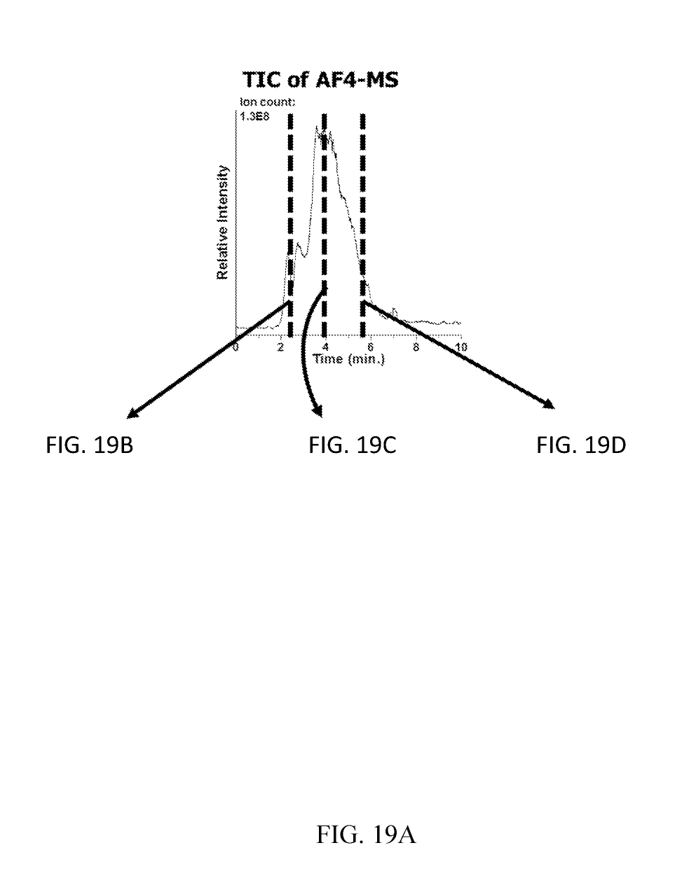

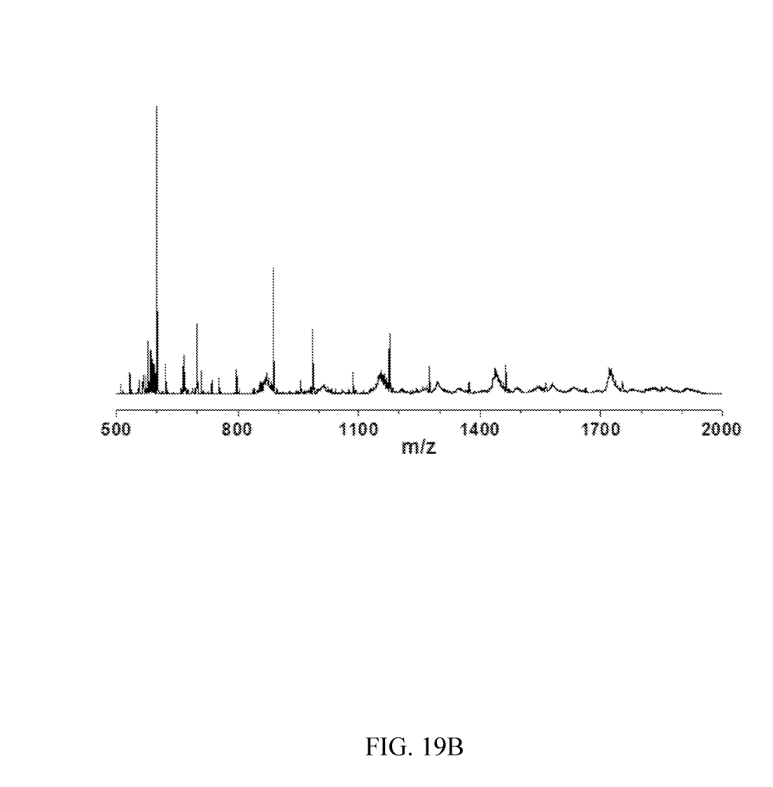

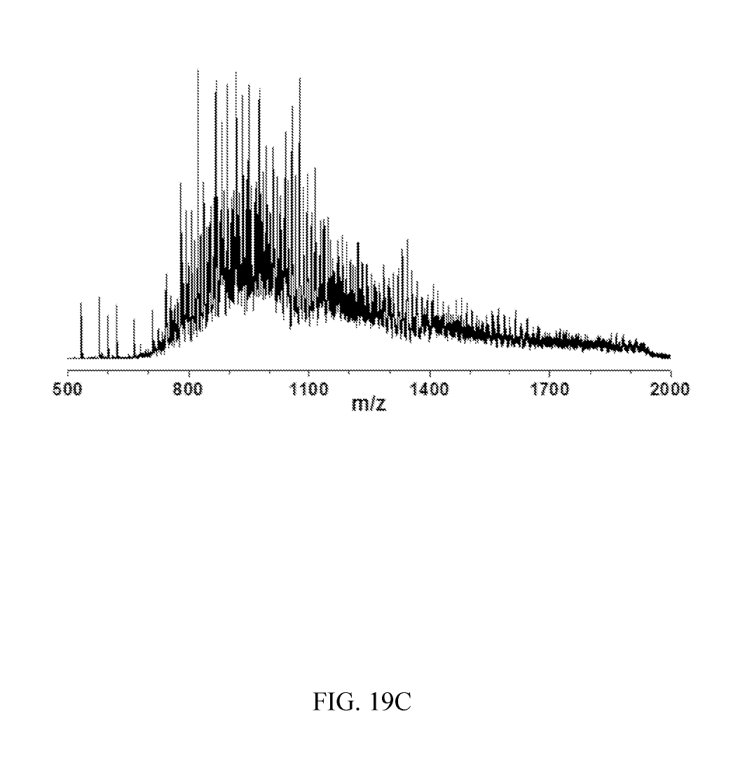

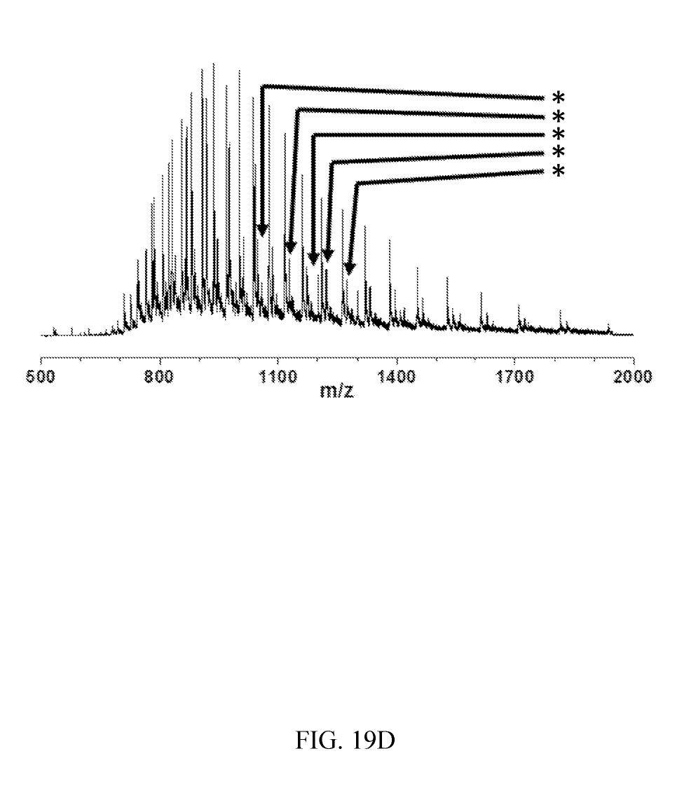

The current channel to remove SDS showed a performance of complete removal within five minutes of focusing/relaxation step and the removal time was evaluated with respect to the intensity of signals from SDS adducts. The effect of SDS removal at different times was evaluated for a total ion chromatogram (FIG. 19A). When removal step was applied for two minutes, SDS removal was not completed and ionization was suppressed at the beginning of the chromatogram (FIG. 19B). However, ionization efficiency was increased and adducts peaks were decreased as retention time goes on (FIG. 19C). At the end of chromatographic peak, clean spectra with small adducts peaks were obtained (FIG. 19D). Thus, sufficient time is required to remove SDS completely and SDS also can removed during the elution steps after focusing/relaxation.

### Example 10

Effect of Flow Rate Following Focusing Time on SDS Removal from Samples Using the Modified AF4 Device.

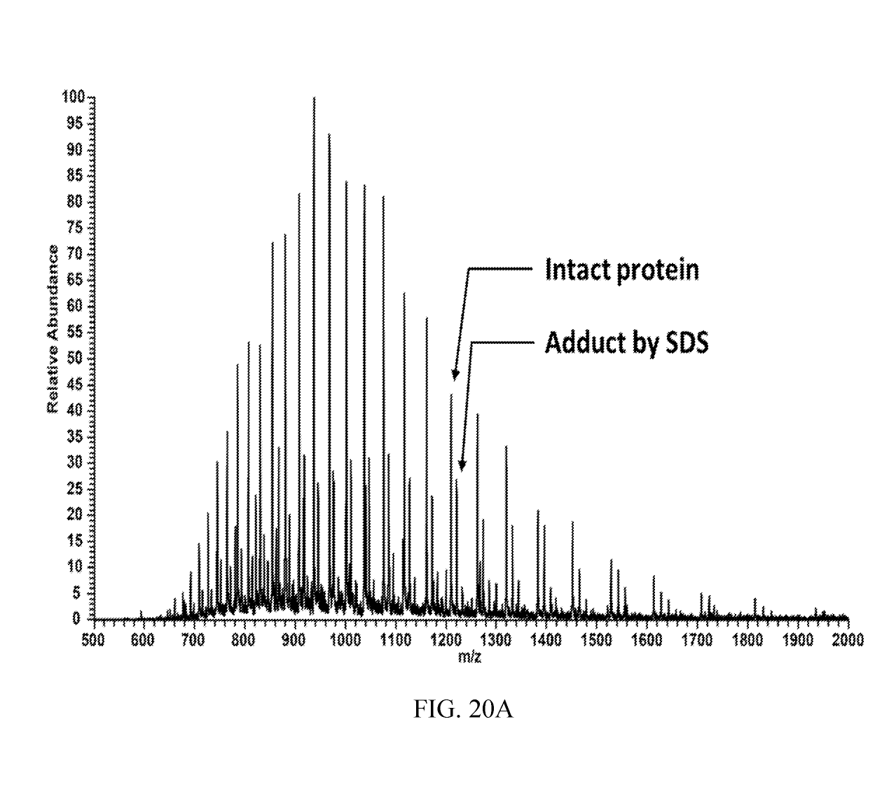

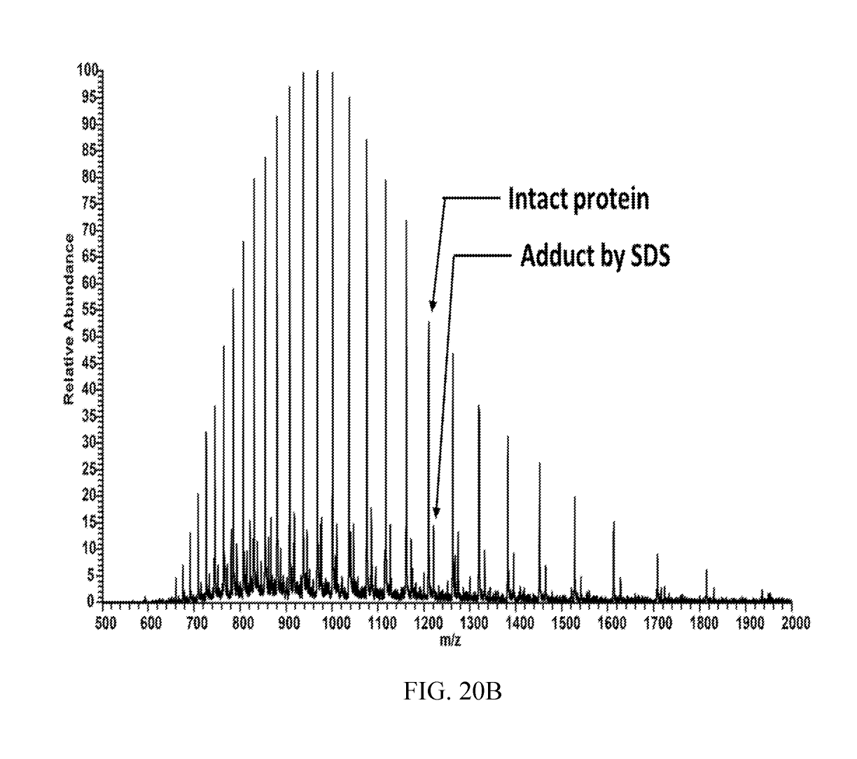

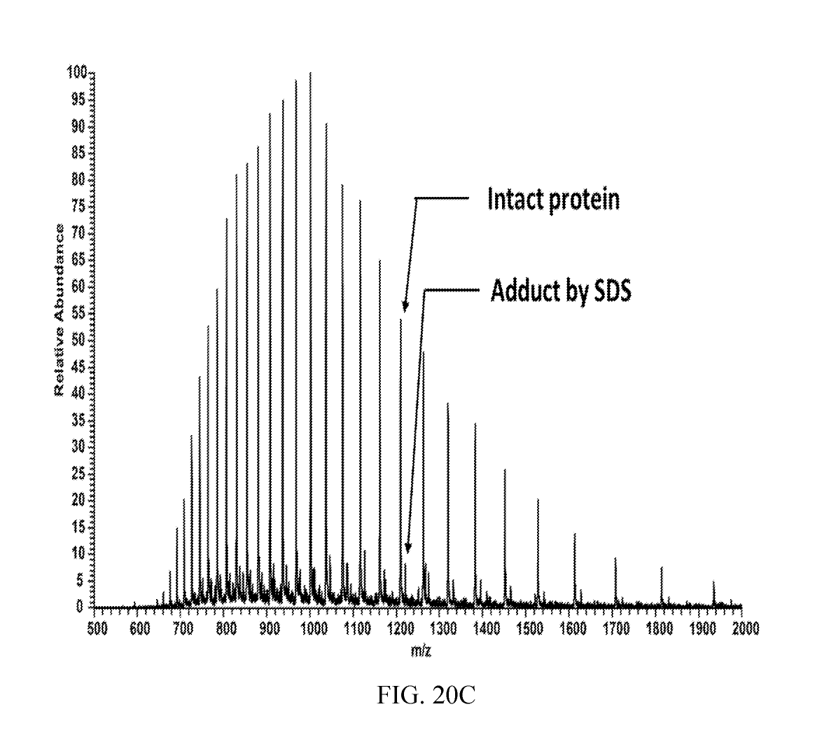

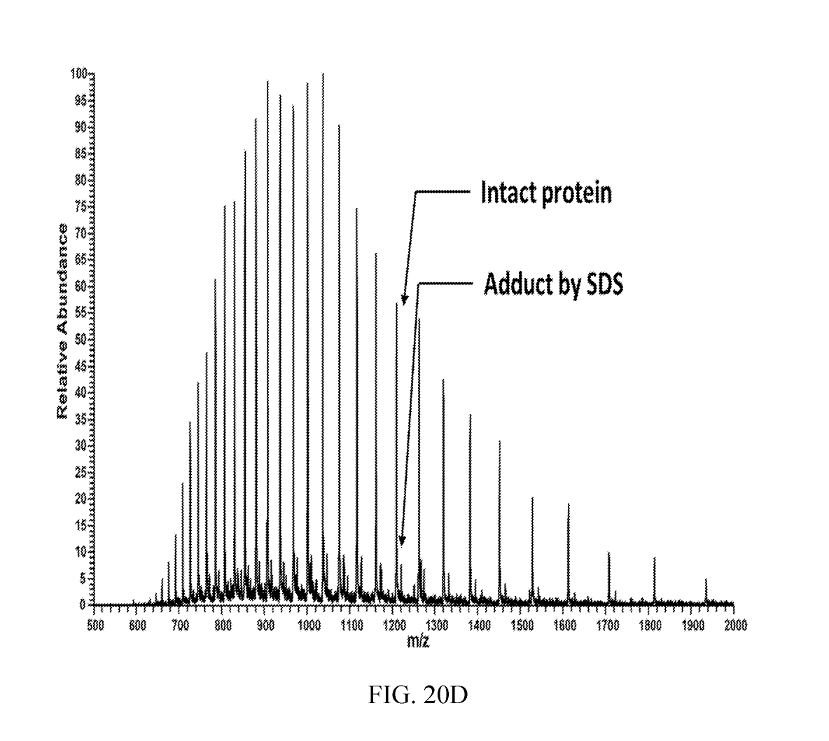

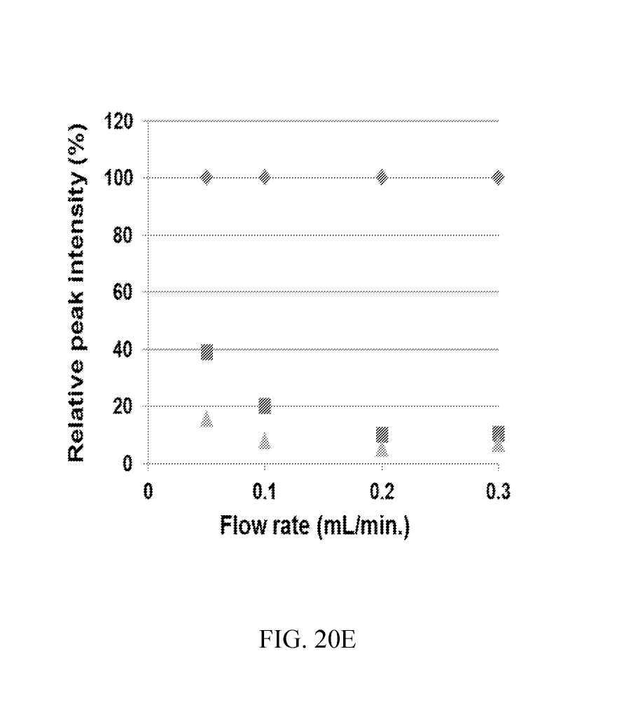

Most of SDS bound protein molecule is removed during focusing step. It is a trapping of proteins in the channel by flow from both ports, large particles such as protein make a narrow band but small molecules such as detergents or salts pass through membrane pores and vented. Therefore, the efficiency or rate of SDS removal is highly dependent to flow rate from pump so the experiments were carried out to compare efficiency in various flow rate. Carbonic anhydrase (1.0 μg) diluted in Tris buffer with 0.1% SDS was used for sample and 30% acetonitrile, 0.1% formic acid and 10 mM ammonium bicarbonate was used for carrier solution. Focusing step was applied for 5 minutes and it is an optimized value from previous result. Each spectrum (averaged ˜15 scans) from the center of Gaussian peak in the fractogram is presented in FIGS. 20A-D.

The SDS adduct peaks were decreased as the flow rate increased for focusing but the intensity of intact protein peaks was not changed significantly. Therefore, the focusing flow rate affected only SDS removal and the recovery of protein was independent to flow rate at the range of 0.05˜0.3 mL/min. The ratio between intact protein and SDS adduct of target ion (+27 ion, m/z 1075.7) is described in FIG. 20E and Table 1, which shows dramatically increasing of SDS removal effect by increasing focusing flow rate.

TABLE 1
Ratio between intact protein and adduct by SDS.

| | Relative peak intensity (%)¹ | | |
|---|---|---|---|
| Flow rate | Target ion: 1075.7 (+27 ion) | | |
| (mL/min) | no SDS | +SDS | +2 SDS |
| 0.3 | 100 | 10.4 | 6.8 |
| 0.2 | 100 | 10.0 | 5.0 |
| 0.1 | 100 | 20.2 | 7.8 |
| 0.05 | 100 | 39.0 | 15.6 |

¹The intensity of intact protein (without SDS) was set to 100% and the relative ratio of SDS adduct was shown. The target ion was set to +27 ion (m/z 1075.7) was showing reproducible intensity at any condition in top 5 peaks.

### Example 11

Detection of SDS by MS Following Negative Ion Mode Focusing

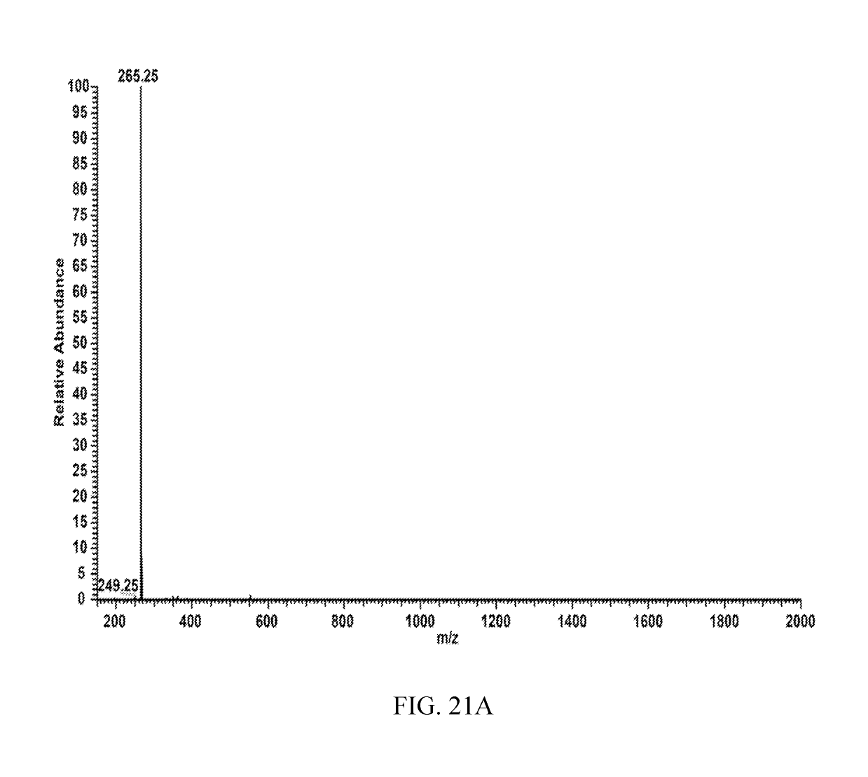

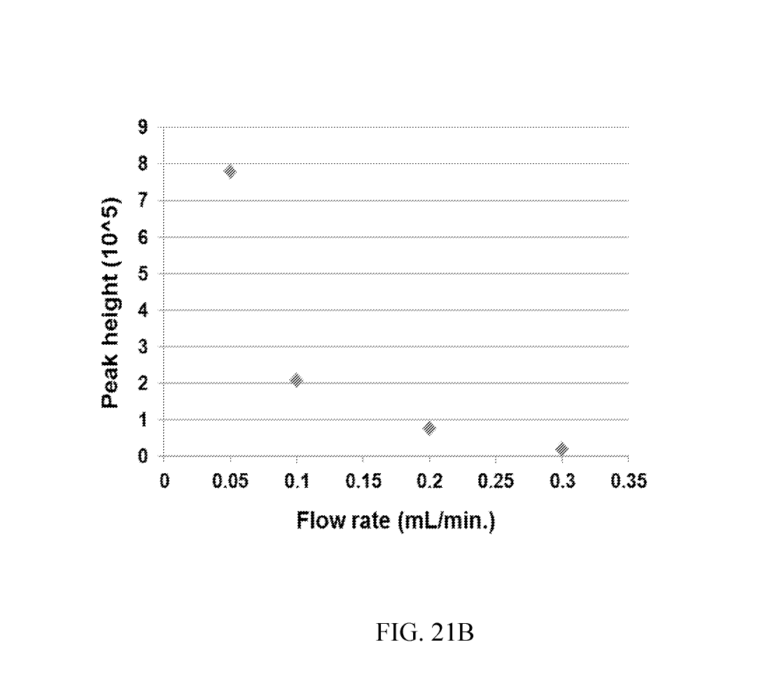

SDS molecule is present with sodium and dodecyl sulfate ions in aqueous solution, but sodium ion is not suitable to observe in typical ion trap mass spectrometry for protein and only dodecyl sulfate anion can be observed in negative ion mode. Therefore, an experiment for detecting the remaining SDS in the sample after the focusing step was performed in negative ion mode, wherein the setup and experimental conditions, including sample volume and carrier solution, were the same as the previous setup. Dodecyl sulfate ion showed very intense peak at m/z 265 at mass spectrum and its intensity was decreased as retention time goes on. An exemplary mass spectrum is described in FIG. 21A. The maximum intensity of dodecyl sulfate ion from each experiment having different flow rates as shown in Example 10 are compared in FIG. 21B and Table 2.

TABLE 2
Intensity of dodecyl sulfate ion and relative ratio of remaining SDS.

| Flow Rate | | |
|---|---|---|
| (mL/min) | Peak Height @ m/z 265 (10\hat{ }5) | Relative Intensity¹ |
| 0.05 | 7.78 | 100 |
| 0.1 | 2.08 | 26.7 |
| 0.2 | 0.76 | 9.8 |
| 0.3 | 0.18 | 2.3 |

¹The peak intensity from first data point (focusing at 0.05 mL/min) was set to 100%.

The other entire spectra from experiments having different flow rates showed similar profiles (though differing in intensity) having only dodecyl sulfate ion. The intensities of the SDS peak differed as a function of flow rate, thereby showing the same trend of SDS as presented in the ratio between intact protein and SDS adduct(s) in Example 10. The remaining SDS after focusing at 0.3 mL/min for 5 min was about 2.3% of the remaining SDS after focusing at 0.05 mL/min for 5 min. Therefore, higher flow rate helps remove SDS from sample solution, as revealed with direct detection of the SDS ion in negative ion mode.

### Example 12

Improved SDS Removal Efficiency by Ionic Strength Increment

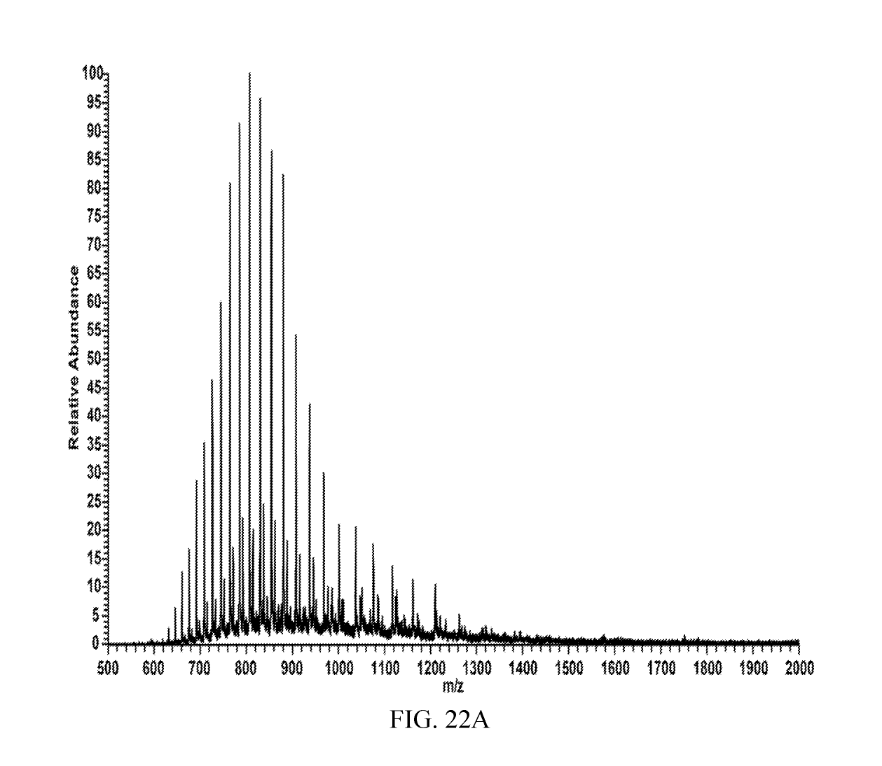

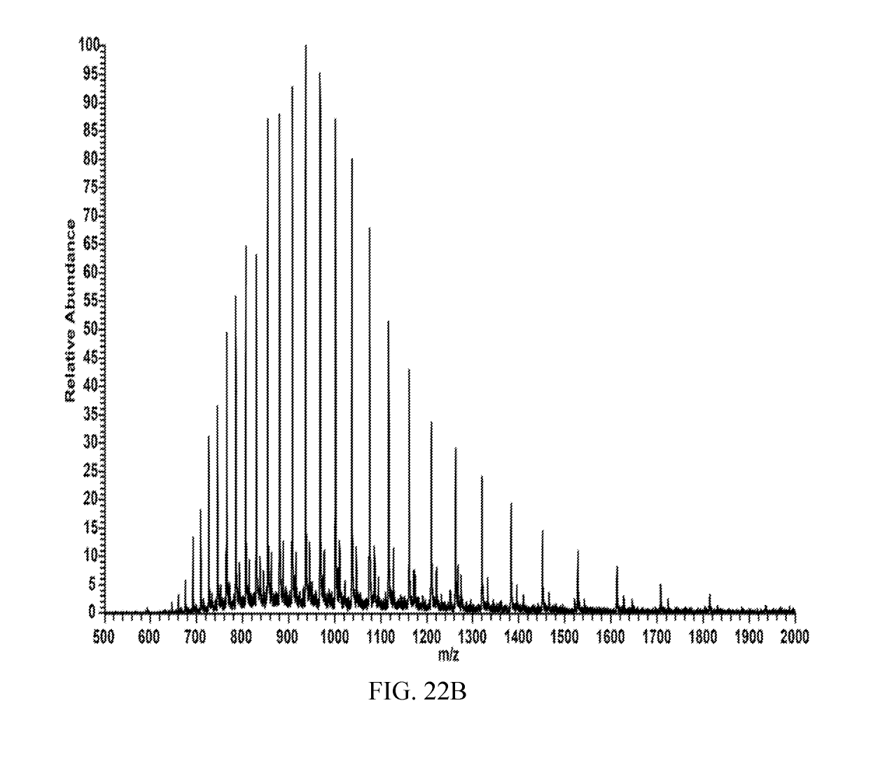

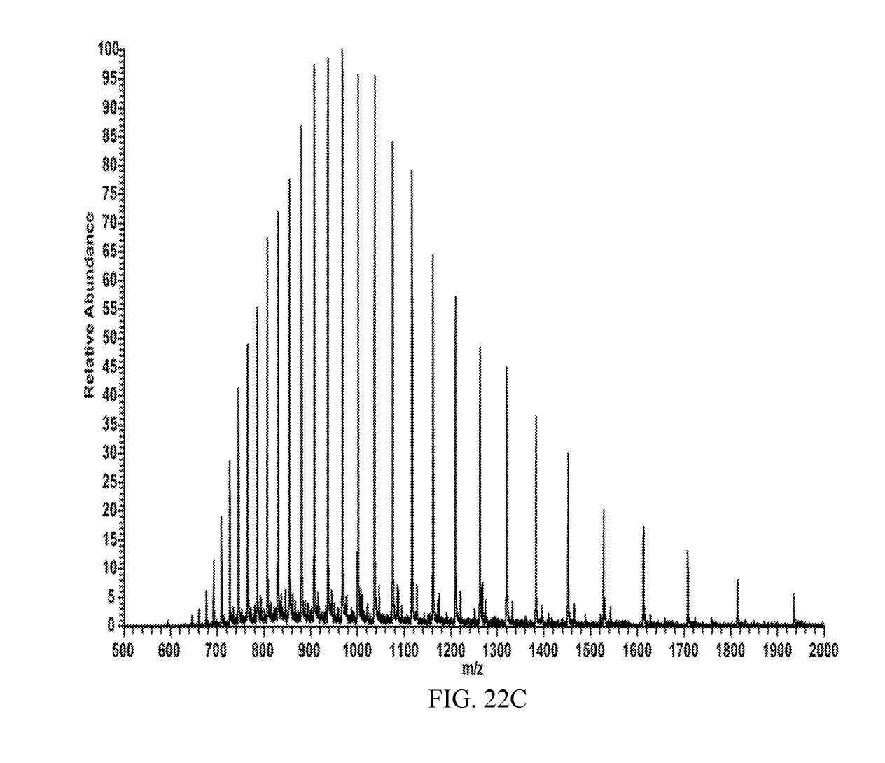

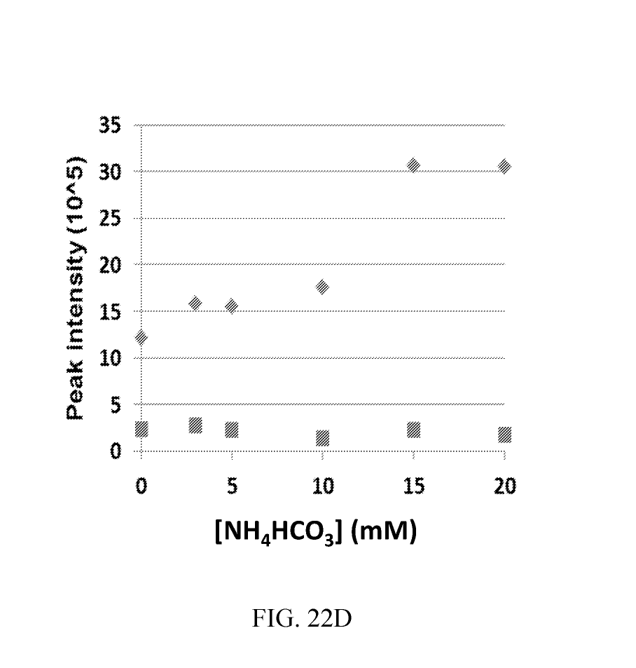

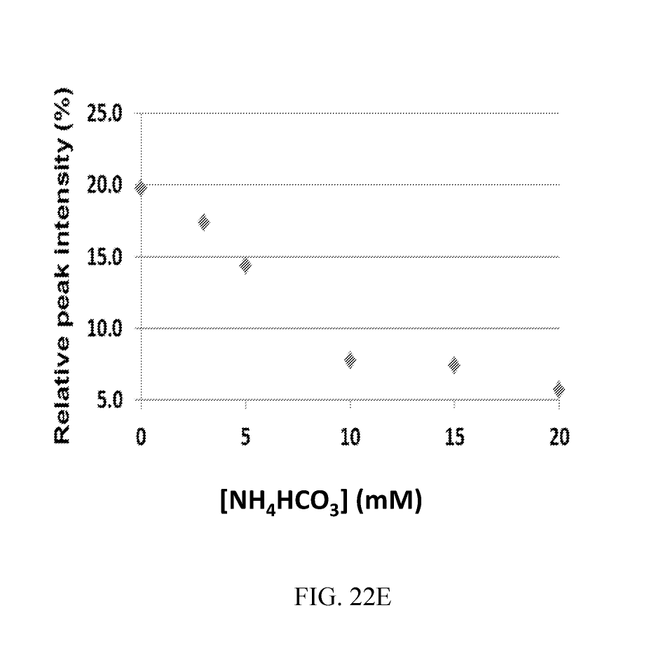

Many kinds of detergent can form a micellear structure above critical micelle concentration (CMC). CMC is dependent to various conditions such as temperature and ionic strength. SDS can also form a micelle, and higher ionic strength (salt concentration) lower its CMC. Therefore, the experiment was carried out to confirm the effect of ionic strength to SDS removal efficiency with removal device and mass spectrometry. Carbonic anhydrase diluted in tris bffer containing 0.1% SDS was used as the sample solution and 0.3 mL/min for 3 minutes of focusing step was applied. The ionic strength was adjusted by increasing the concentration of ammonium bicarbonate and 0, 3, 5, 10, 15, 20 mM of ammonium bicarbonate (final concentration) was mixed into the carrier solution. FIG. 22A-C shows representative mass spectra at the center of a Gaussian peak in each fractogram.

The SDS adduct peaks were decreased as ammonium bicarbonate concentration increased (compare FIGS. 22B and C to FIG. 22A). Without the invention being limited by any particular theory, SDS might competitively partition between micelle assembly and protein binding, wherein SDS would tend to make more micelle structures at lower CMC. Therefore, less SDS would bind to protein surface and the ratio of adduct may be decreased as ammonium bicarbonate concentration increased. Each mass spectrum shows different charge distribution of carbonic anhydrase owing to pH changes due to addition of ammonium bicarbonate; therefore, the base peak, which has different m/z in each spectrum, was compared to characterize removal efficiency. The peak intensities and SDS adduct ratio are presented FIG. 22D and Table 3, while FIG. 22E shows the trend of decreasing SDS adduct:protein ratio as a function of increasing ionic strength in the carrier medium.

TABLE 3
Ion intensity of top peak and its SDS adduct in each spectrum.

| NH₄HCO₃ | | | | |
|---|---|---|---|---|
| Concentration | Most intense peak¹ | Peak intensity (10\hat{ }5) | | Peak Ratio² |
| (mM) | (m/z) | no SDS | +1 SDS | (%) |
| 20 | 968.2 | 30.5 | 1.7 | 5.7 |
| 15 | 968.2 | 30.6 | 2.3 | 7.4 |
| 10 | 968.2 | 17.6 | 1.4 | 7.7 |
| 5 | 937.1 | 15.5 | 2.2 | 14.3 |
| 3 | 907.8 | 15.8 | 2.7 | 17.3 |
| 0 | 807.1 | 12.1 | 2.4 | 19.8 |

¹The charge distribution is changed as pH changes so base peaks having different m/z at each spectrum were compared for SDS removal efficiency.
²Peak ratio (%) defined as ratio of +1 SDS adduct peak intensity/Protein peak intensity, multiplied by 100.

### Example 13

Effect of Outflow Rate with Further Improvements in Spacer Design

The use of very slow outflow rates (less than 1 μL/min) for electrospray ionization is required for efficient and stable ionization of proteins and it can cause excessively long retention time and analysis time. Moreover, higher ion signal improves fragment ion generation in the mass spectrometer for identification or characterization, so the final results from a complex mixture can be highly dependent to flow rate. Therefore, to minimize retention time, further miniaturization of the AF4 channel by decreasing channel thickness can be used. In this approach, an inner tip-to-tip length was adjusted to 3.0 cm from original length of 7.0 cm to reduce the channel volume by ⅓ (FIG. 23A). Channel length is not directly related to retention time in the equation, but the cross flow rate can be reduced as decreasing of the area of membrane surface. Therefore, the ratio of inflow/outflow can be changed and retention time would be reduced.

An additional nanoport for sample injection was used to improve removal of matrix such as SDS. In current technique, removal step is carried in focusing/relaxation step and that is the flow of carrier solution migrated into specific position in the channel from both of two ports. Therefore, proteins make a narrow band at the position and then actual removal of SDS is performed at the band. However, the filtration efficiency depends on the area of membrane surface, the removal speed can be limited. The additional port located at the center position maximized the SDS removal efficiency. All of exterior design components, such as the stainless steel plate and frit were changed to accommodate the new spacer design. The material of new spacer was changed to polyether ether ketone (PEEK) to provide improved chemical resistance (see Example 14). When the sample solution is injected via the center port, sample solution may spread on the membrane radially so filtration can be occurred in the wide area. The protein is migrated into a band at the center position on the channel by the pump flow after the removal step. The high-resolution separation of proteins is carried out prior to this technique so the separation based on AF4 mechanism is not highly required. A comparison of mass spectra from samples processed at different outflow rates of 5.0 μL/min and 2.5 μL/min is illustrated in FIGS. 23B and C, respectively.

### Example 14

Effect of Space Material Composition on MS Product Analysis

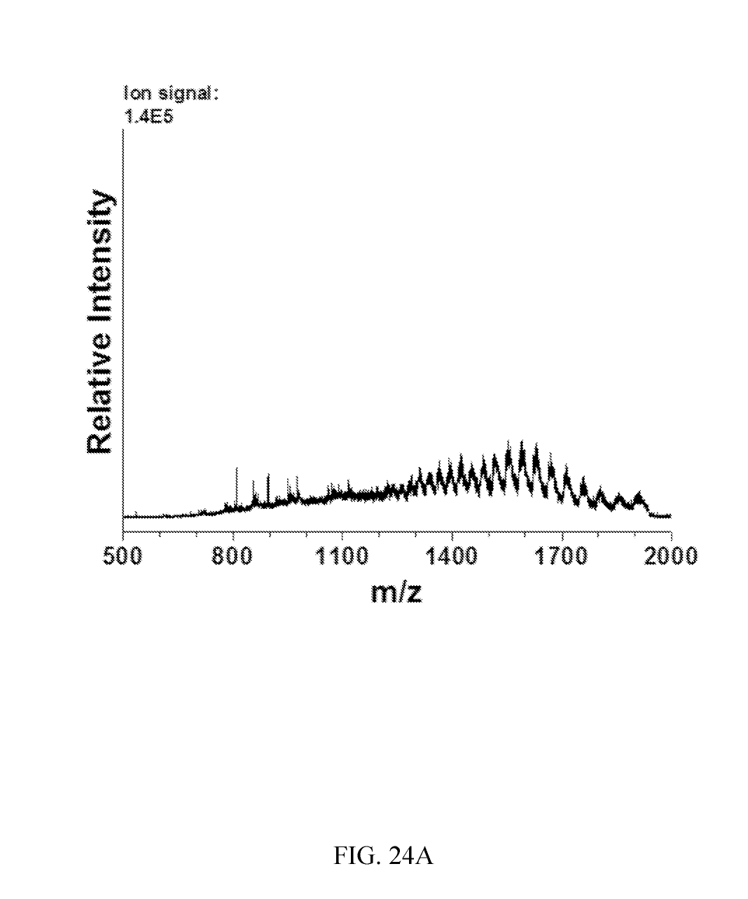

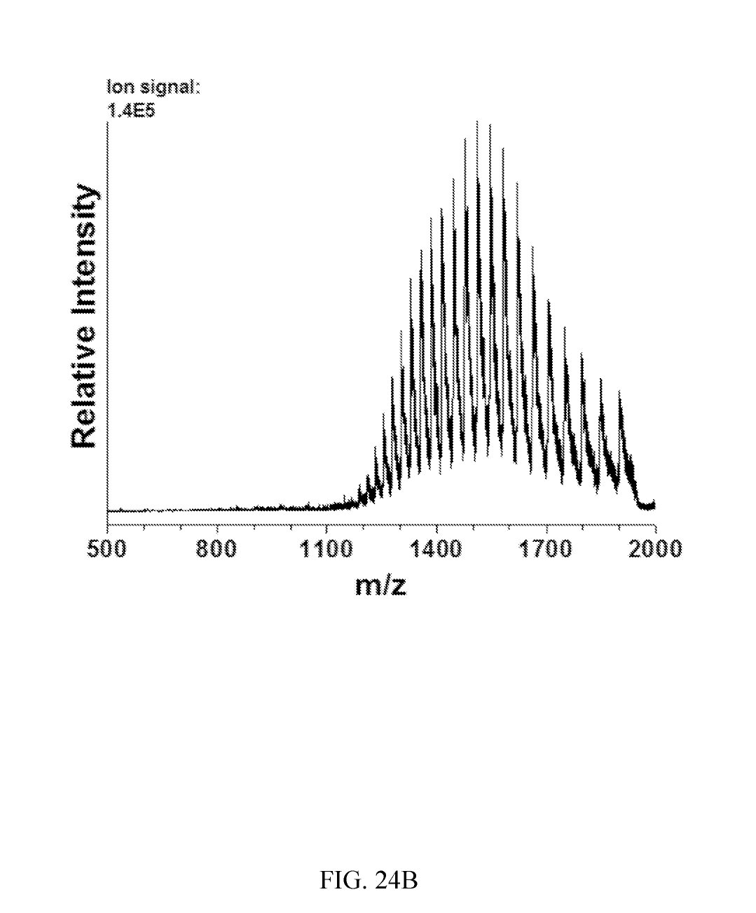

The presence of organic solvent such as acetonitrile is required for electrospray ionization. The ionization of protein is occurred in the droplet at the end of emitter tip and fast evaporation rate (desolvation) of solvent is critical to efficient and stable ionization so water/acetonitrile or methanol mixture is widely used at ESI. However, current AF4 technique uses the acryl, polyethylene terephthalate (PET) or polycarbonate plastics and its chemical resistance is not suitable to use organic solvent so organic solvent is mixed separately right before introducing to mass spectrometry using an external syringe pump. The new spacers described herein are made from polyether ether ketone (PEEK) film that has an excellent chemical resistance so pre-mixed solvent (aqueous buffer and acetonitrile) can be applied directly to the channel. Mass spectra from spacers made from the different materials are presented in FIGS. 24A,B.

### Example 15

Improved AF4 Designs Providing Leak-Free Sample Processing

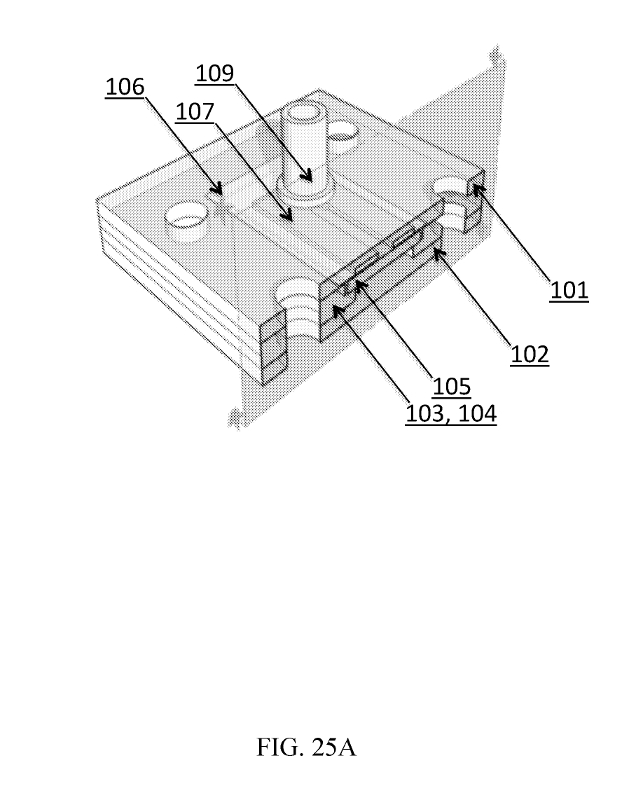

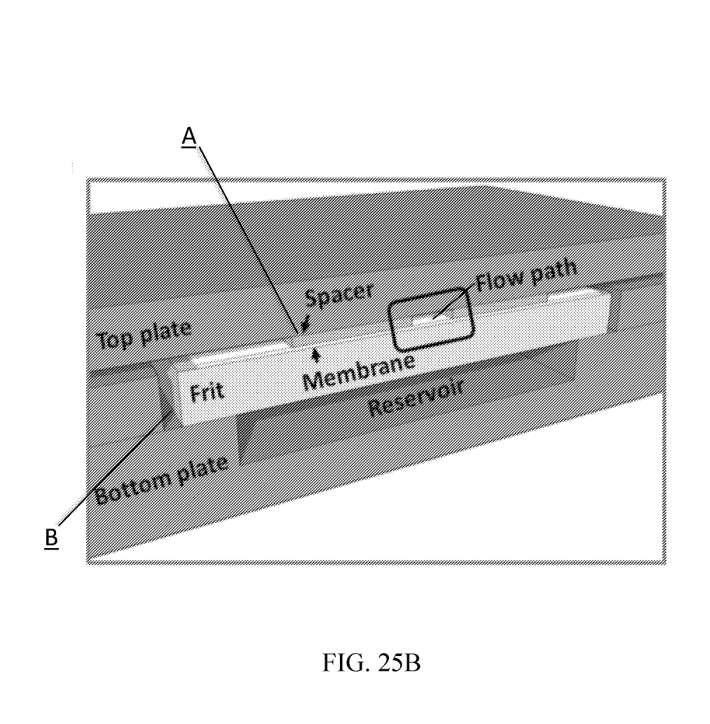

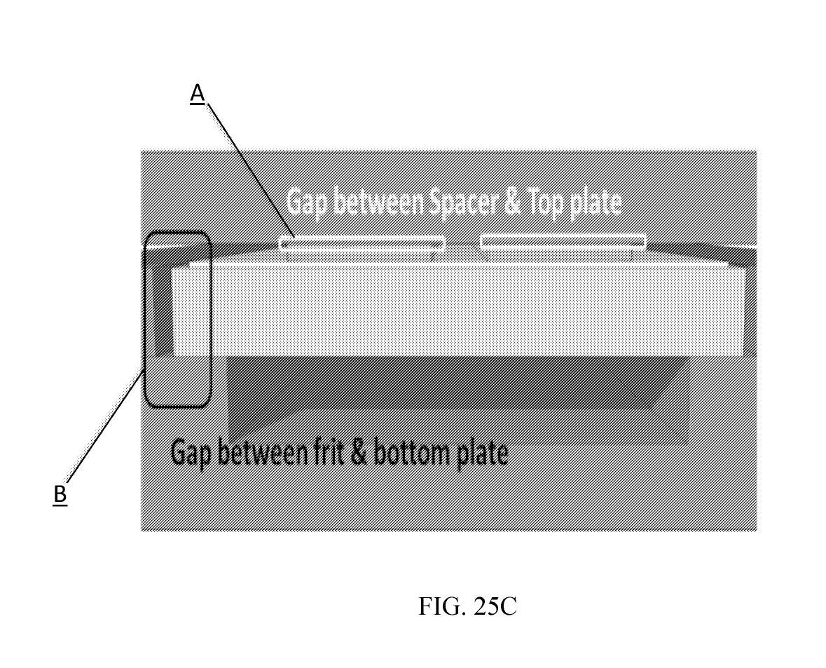

Current design of removal device shows some loss of proteins during a cleanup process and that would come from various reasons. A leakage of carrier solution is also one of major reasons, which could be serious obstacle for reproducible removal process. Referring to FIG. 25A, a portion of AF4 device 100 is illustrated, wherein top clamping plate 101, bottom clamping plate 102, reservoir plate 103, frit holder plate 104, frit 105, membrane 106, spacer 107 and flow inlet 109 are shown. Most leakage occurs in the gap A between spacer 107 and top clamping plate 101 and the gap B among bottom plates 102, 103 and 104 in FIGS. 25B,C.

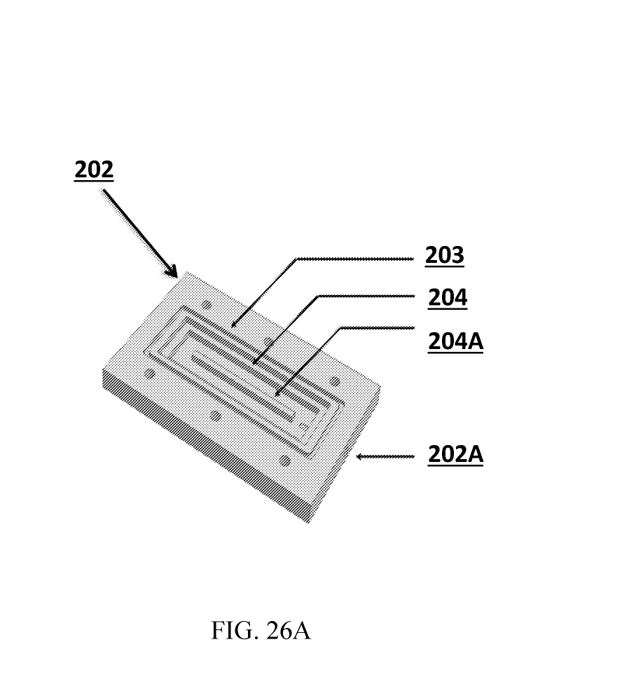

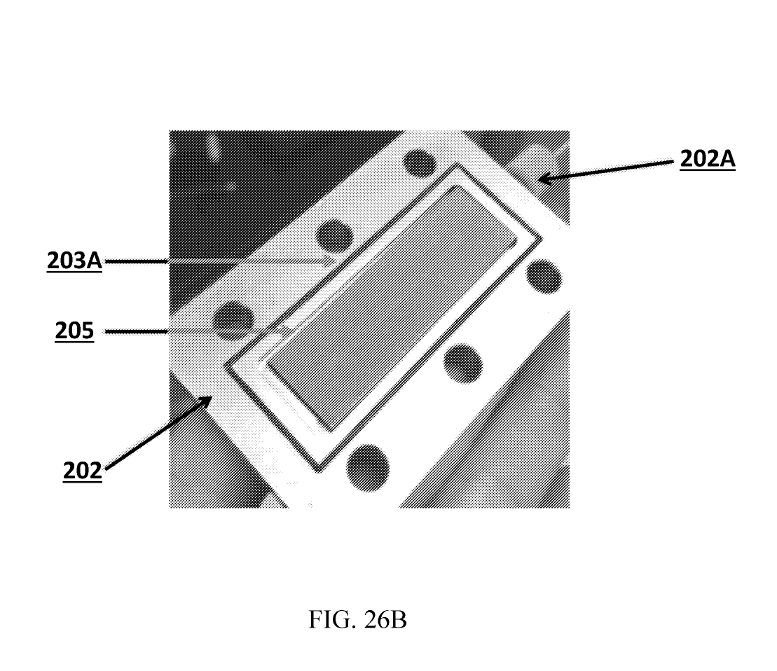

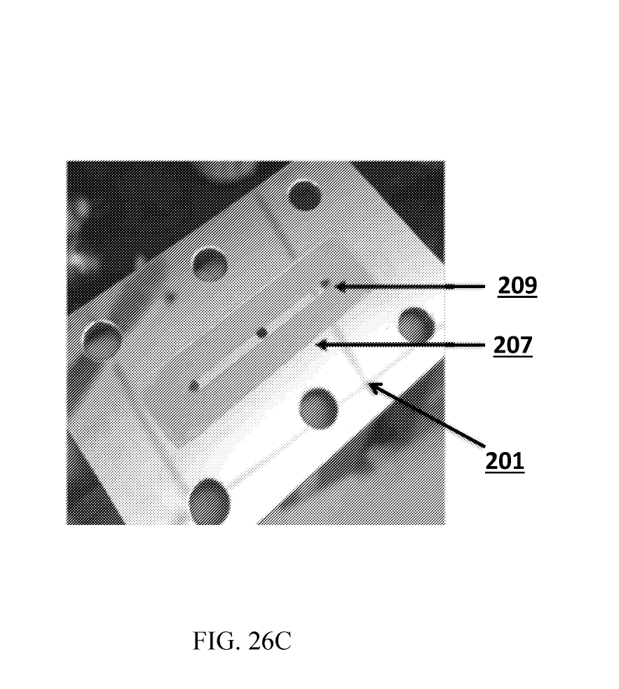

Referring to FIG. 26A, in another aspect of AF4 device 200, a design can have as few as two plates for the complete assembly (top plate 201 and bottom plate 202). Bottom plate 202 can made by engraving a plate to include a plurality of slots. An outer slot 203 provides a slot for an O-ring 203A or other similar gasket material to provide a leak-free seal when upper plate 101 and bottom plate 102 contact in the completed assembly. An inner slot 204 provides a housing for holding frit 205. An optional supporting bar 204A can be provided for adjusting the height of frit 205. Bottom plate 202 integrates the features of bottom plate 102, reservoir plate 103 and frit holder plate 104, as depicted in FIG. 12. Bottom plate 202 also includes an integrated crossflow outlet port 202A.

Referring to FIG. 26B, a prototype bottom plate 202 is illustrated, showing the locations of the crossflow outlet port 202A, O-ring 203A and frit 205. Referring to FIG. 26C, a prototype top plate 201 is illustrated, showing the locations of the spacer 207 and one of the plurality of flow inlets 209. The material of spacer 207 can be a PEEK tape with silicon adhesive for sealing the gap so all leakage can be prevented. Furthermore, PEEK provides superior chemical and thermal resistance than other types of spacer 207 compositions (see, e.g., Example 14). In addition, PEEK tape can provide spacer 207 having a thickness of about 0.012 mm (12 microns).

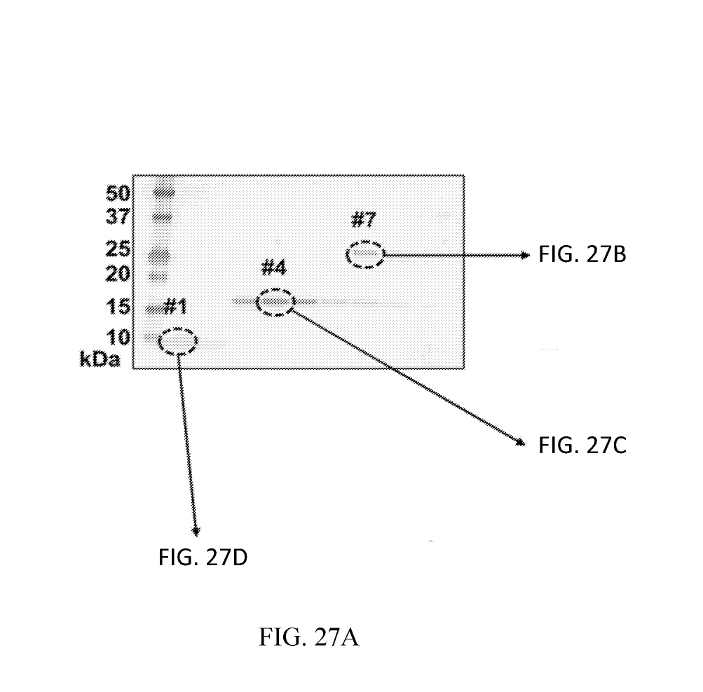

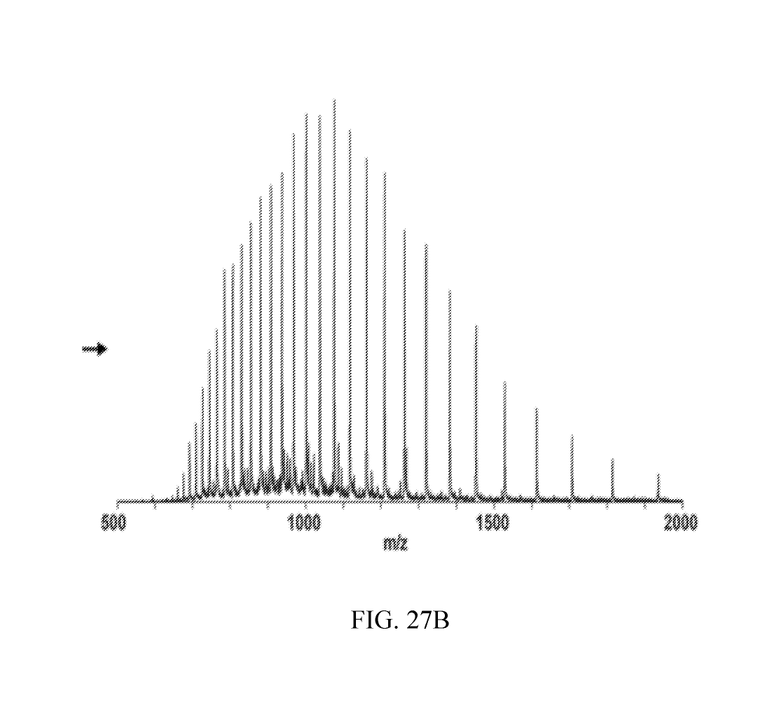

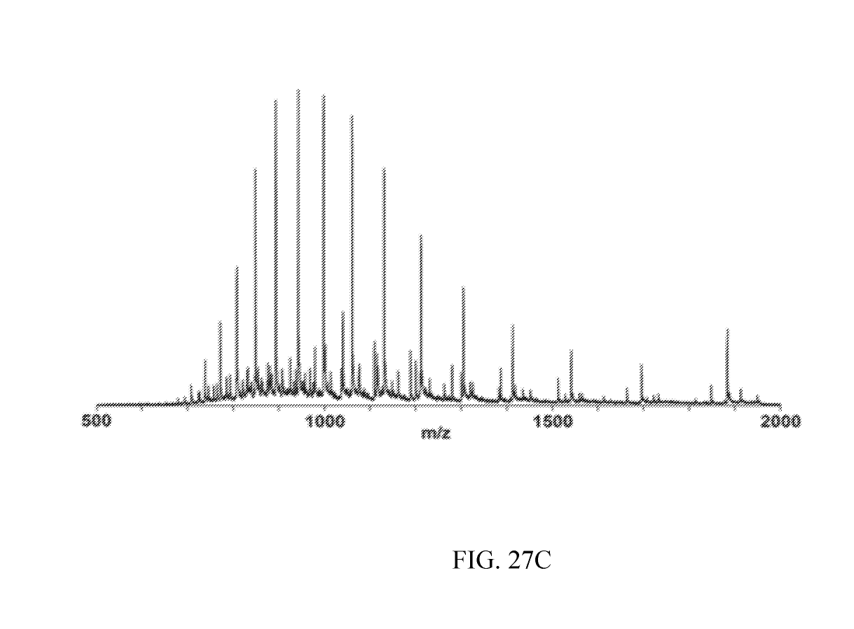

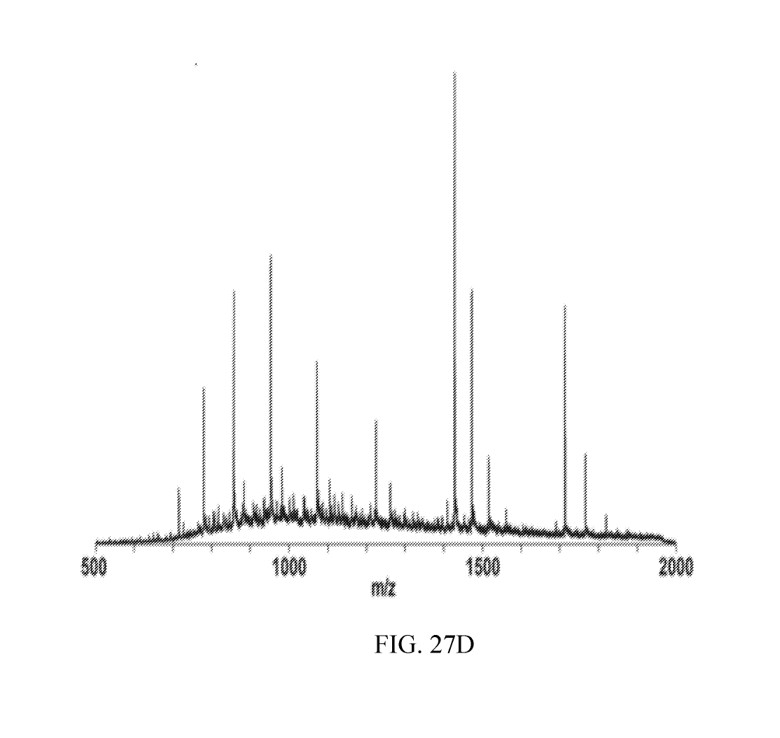

To evaluate the recovery of a prototype of removal device 200, a comparison of peak intensity was performed between classical precipitation method and the in-line removal method after gel-based separation. Ten micrograms of each protein was fractionated in a GELFrEE cartridge for gel-based separation and its separation result is shown in a slab gel image in FIG. 27A.

For the in-line removal method, 5 μL each protein-containing fraction was injected into the removal device and analyzed via mass spectrometry without any treatment. The three spectra of purified proteins carbonic anhydrase, myoglobin and ubiquitin by online cleanup with the removal device are shown in FIGS. 27B-D. While small SDS adduct peaks are visible for ubiquitin and myoglobin, the spectrum of carbonic anhydrase shows no remaining adducts.

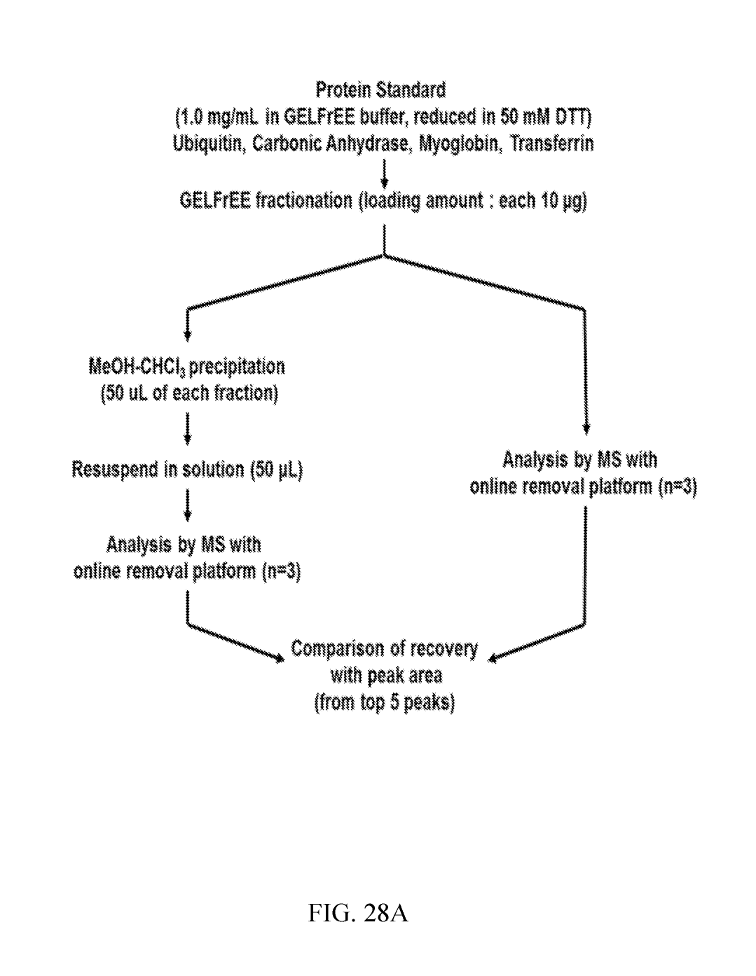

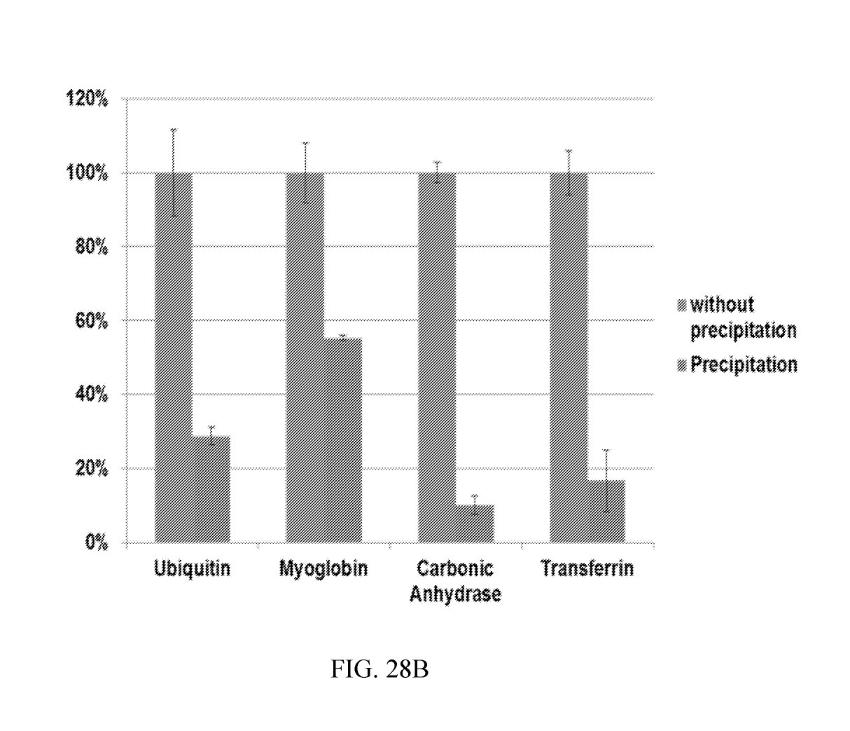

For purification by classical method, 50 μL of each fraction was precipitated using MeOH/CHCl₃and re-suspended pellet was also injected into the removal device and analyzed via mass spectrometry. The procedure is presented diagrammatically in FIG. 28A. Four standard proteins (ubiquitin, myoglobin, carbonic anhydrase and transferrin) in a mixture were separated via GELFrEE. The precipitation procedure was performed and the resultant pellet was resuspended. Both precipitated and unprecipitated samples were then subjected to in-line detergent removal. The analysis was performed in technical triplicate for each sample. And those peak areas were used to quantitate recovery.

The quantitative analysis for recovery was carried out by comparing extracted ion chromatograms, which were area of top 5 peaks by intensity for each species. The histogram of peak area comparison is shown in FIG. 28B. The peak area of GELFrEE fractions purified by the in-line method was set to 100% because the in-line removal method always gave higher recovery than the precipitation method and measured peak areas were very different for each protein. Carbonic anhydrase showed the largest difference, suffering an almost 10 fold reduction compared to the precipitation method. In the case of ubiquitin, myoglobin and transferrin, the on-line method showed 2 to 6 fold higher recovery. The difference in recovery is highly dependent on the protein species and there are no obvious trends from this limited sample set.

### Example 16

Applications with Biological Samples

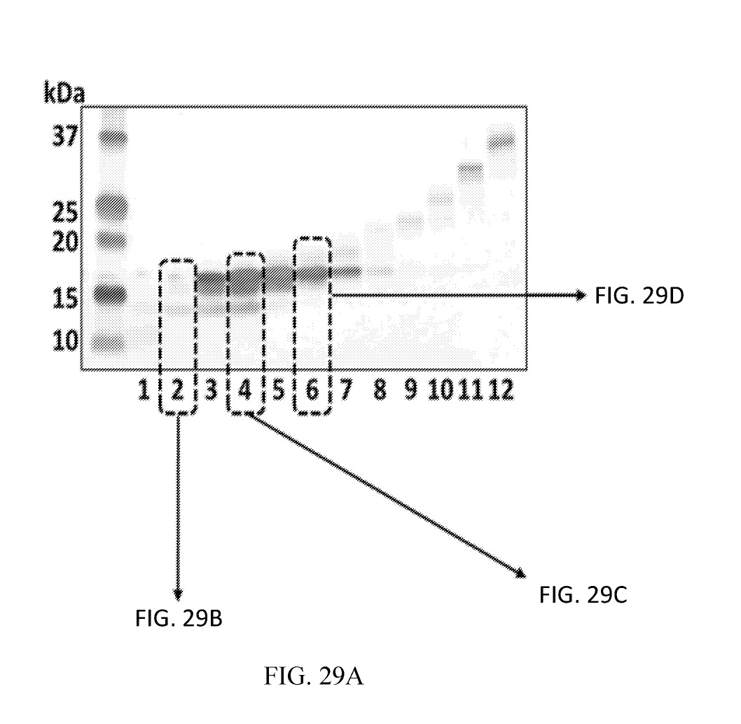

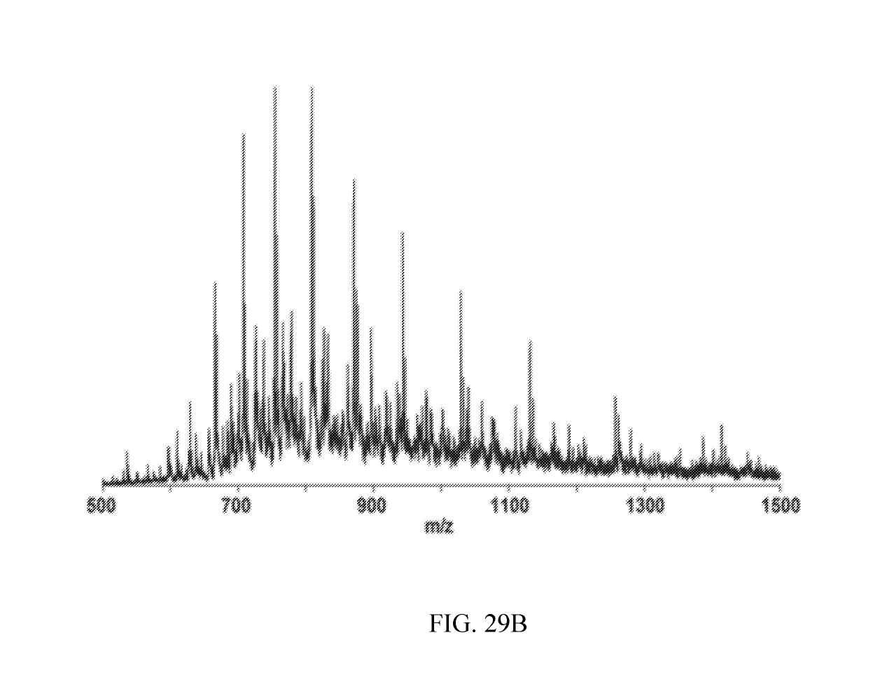

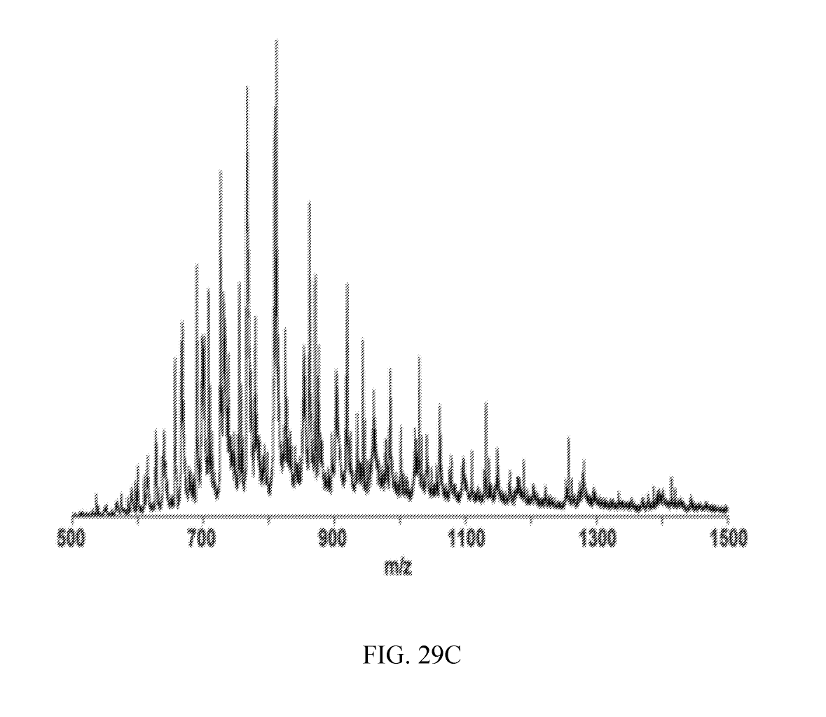

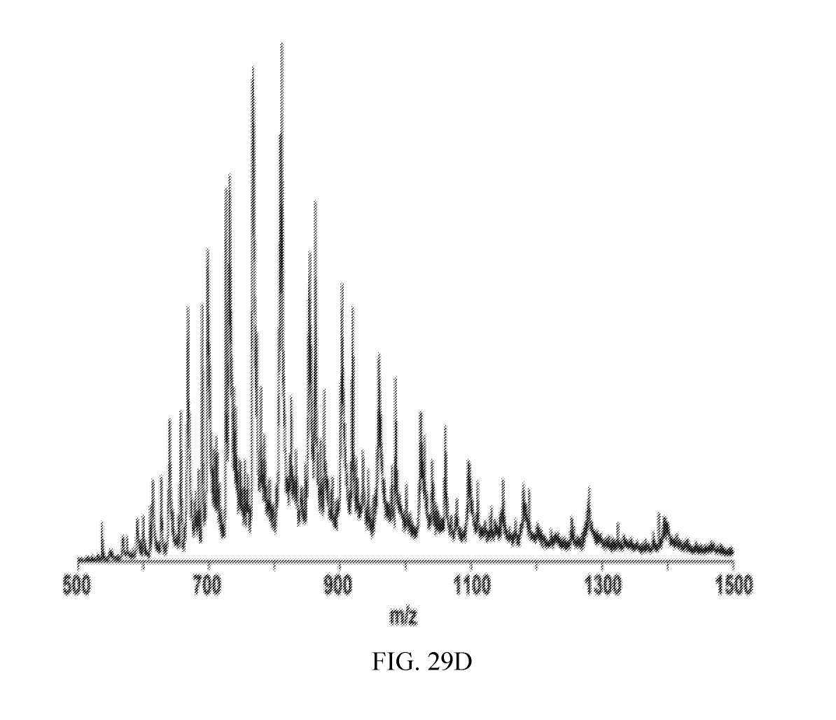

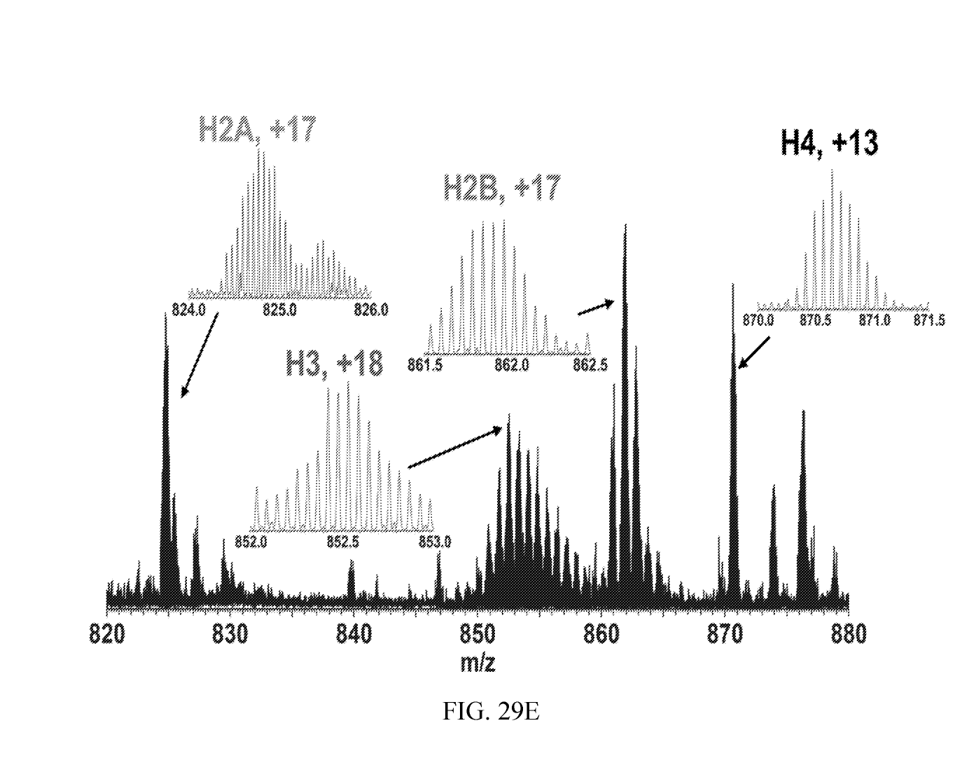

To demonstrate the platform utility with protein mixtures derived from biological samples, an acid extract of nuclei from HeLa S3 cells was fractionated with gel-based separation, generating a sample set with relatively low complexity. We were able to identify and characterize the modified proteins within the samples without further separation owing to the simplicity of the samples. The GELFrEE result was visualized with an SDS-PAGE slab gel followed by silver staining (FIG. 29A). Each 5 μL of 150 μL in fraction was injected into the removal device and it was purified and then analyzed by a mass spectrometry. The MS1 spectra from fractions #2, 4, and 6 are depicted in FIGS. 29B, 29C and 29D, respectively, permitting identification of the species based upon molecular weight. In the MS1 spectrum from each fraction, there is some overlap of two or more histone proteins, but the most abundant peaks from each spectrum show strong agreement with the slab gel image.

Fraction #7 was analyzed by a high-resolution mass spectrometer (Q Exactive HF Orbitrap) and the results are described in FIG. 29E. All four core histones were identified by accurate intact mass (<5 ppm). Despite the presence of various histone proteoforms, each isotope of histone species was resolved, which enabled direct determination of molecular weight without any further calculation. This experiment is the first attempt to analyze histone subunits without any chromatographic separation in top-down proteomics research. It indicates that the in-line removal device is useful to purify proteins from matrix including SDS for direct analysis by electrospray mass spectrometry.

### Example 17

On-Line Hyphenation to Gel-Based Separation.

A total on-line system for the separation and analysis of protein samples in the following manner. A matrix processing module was coupled (that is, in fluid communication) between a protein separation module and a mass spectrometer module. The protein separation module used in this aspect was a gel-based separation device. The customized protein separation module enabled collection and delivery of liquid-phase fractions to the matrix processing module using flow control from an in-line pump. All components of the protein separation module were assembled with general fittings typically used for chromatography (for example, HPLC fittings and connectors). The protein separation module included a gel hand-casted in a column composed of glass tubing, a buffer chamber and electrodes (FIG. 30A). The cathode was connected to the cathode buffer reservoir at the top of the gel column using HPLC fittings, and the anode was connected to a small anode buffer reservoir at the bottom of the gel column using similar HPLC fittings. A frit-in-a-ferrule (IDEX CORP. (Lake Forest, Ill. (USA))) was installed below the anodic buffer chamber to prevent such interference with separation due to gravity-induce flow from the anode reservoir. The tubing for connecting the gel column to the anode chamber served as a collection chamber for protein fractions. A syringe pump was used to control flow at a user-specified rate (i.e., 0.2 mL/min) to migrate proteins eluted from the gel column to retention chamber for further purification. A preferred retention chamber in this system was a sample loop having a volume from about 0.01 mL to about 0.50 mL. A preferred sample loop as a retention chamber used in this particular aspect had a volume of about 0.20 mL. An in-line 6-port valve was implemented in the system to control conveyance of flow containing the protein fraction(s) from the protein separation module to the matrix processing module via action of the HPLC pump. This system bypass permits concurrent operation of the different modules operating at different backpressures. The matrix processing module operates under ˜100 psi but this pressure, which exceeds the operating pressures of the protein separation module, which operates at atmospheric pressure conditions. The electrophoresis carrier medium of the protein separation module was based upon a Tris buffer system similar in composition typically used in SDS-PAGE processes. Likewise, input sample for the protein separation module was prepared and applied in the gel column of the protein separation module in a manner similar to that used for conventional SDS-PAGE (that is, samples were loaded directly onto the gel of the protein separation module using a pipette).

FIG. 30B depicts a prototype protein separation module in operation, whereby multiple bands of standard protein marker (from Bio-Rad) are separated. The first band is bromophenol blue in sample buffer and its retention time was approximately 30-35 min when 800 V of voltage was applied to 5 cm of 2-10% gradient gel column. The elution of the first band was observed at the end of gel column, where after fractions were collected at 5 minute-intervals.

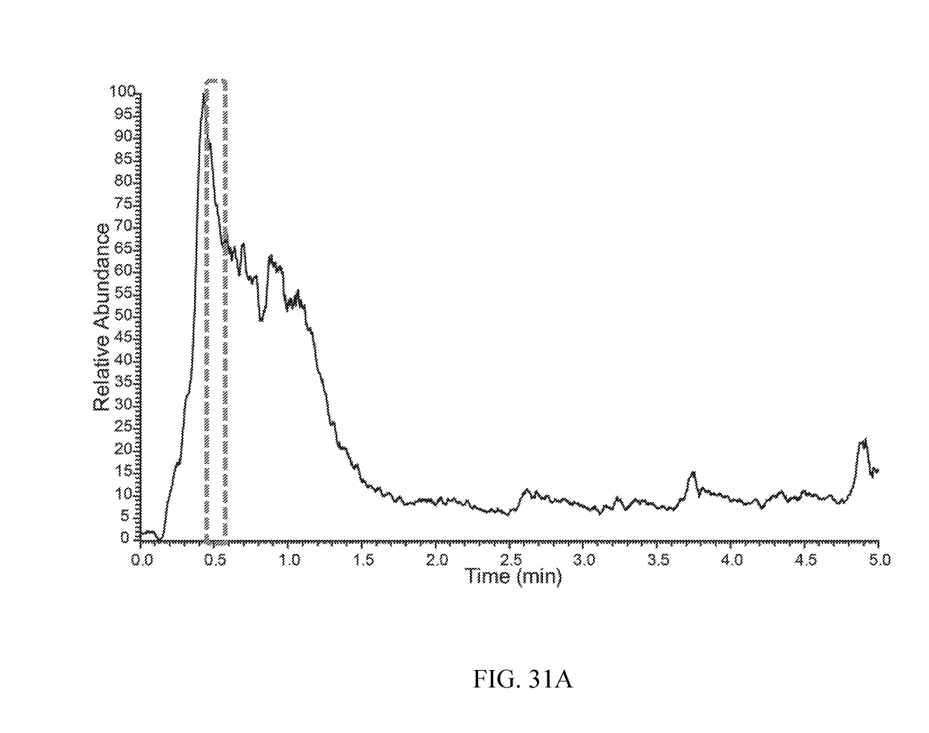

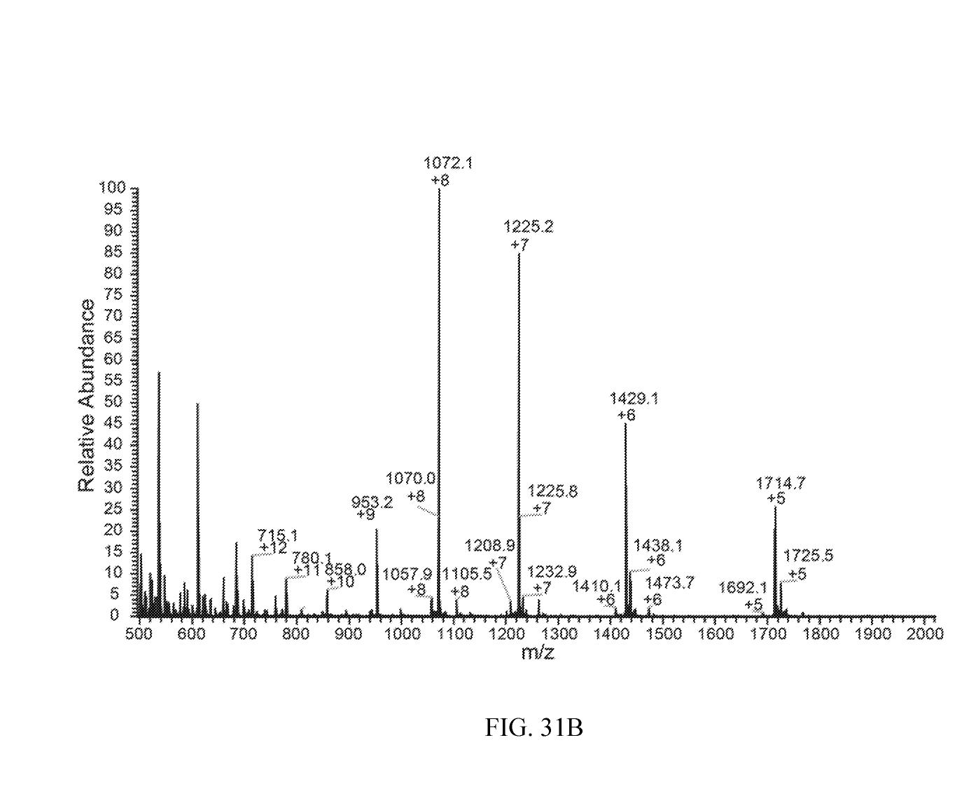

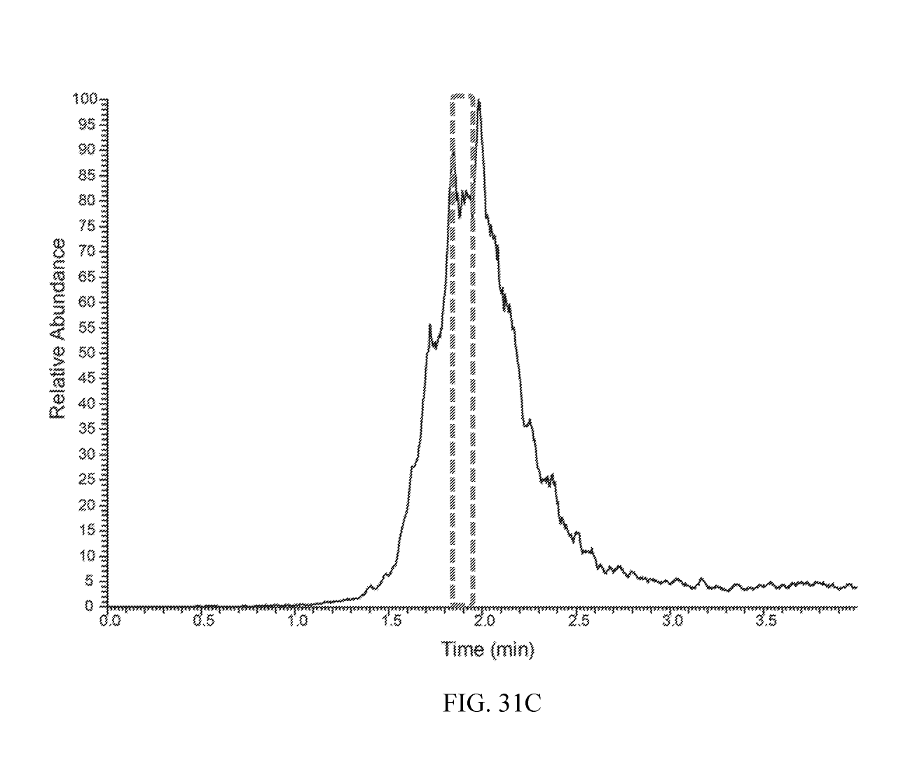

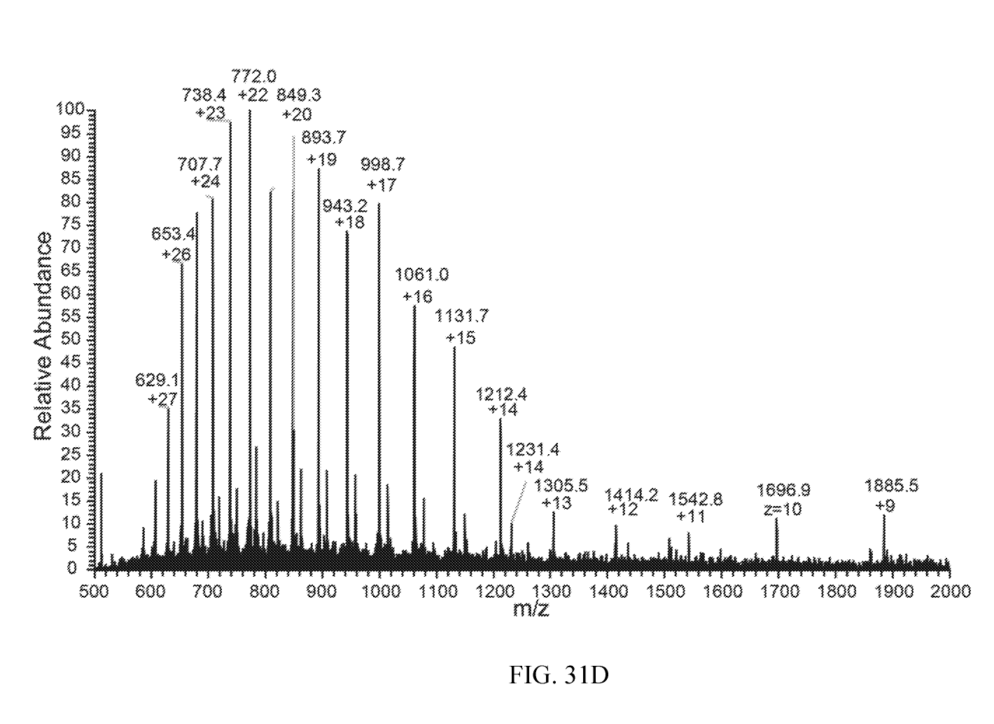

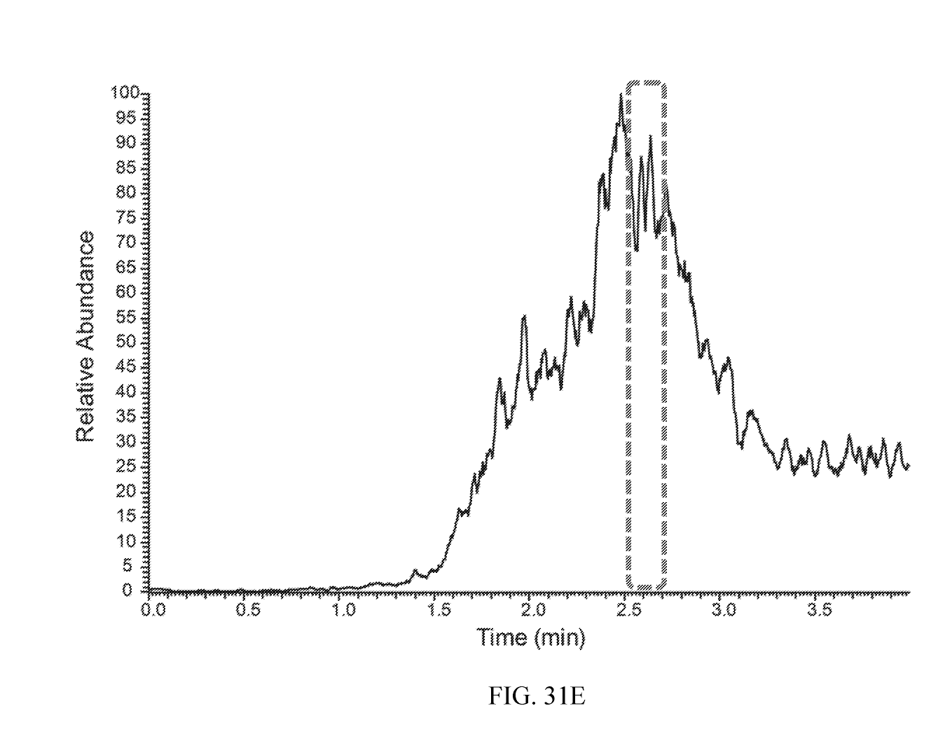

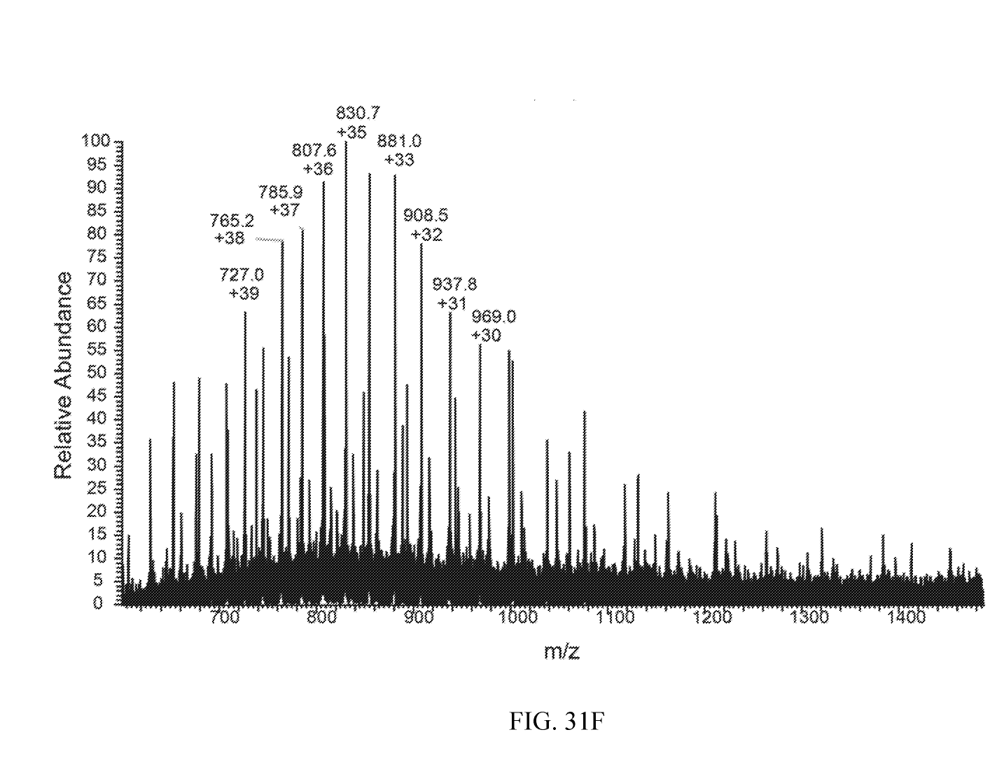

A mixture of three protein standards (1 ug each of ubiquitin, myoglobin and carbonic anhydrase) was loaded in polyacrylamide gel column of the protein separation module. Each protein was separated in the gel column of the protein separation module; fractions were collected and moved to the matrix processing module; finally, purified proteins from the matrix processing module were analyzed by high-resolution mass spectrometry in the mass spectrometer module. FIG. 31A shows the extracted chromatogram of the base peak of ubiquitin. Ubiquitin was collected from first fraction (35-40 min for gel-based separation time) and removal step was carried out for 5 min. There is no significant adduct peaks in the spectrum and direct identification of molecular mass was available due to isotopic distribution with high resolution as shown in FIG. 31B. Myoglobin and carbonic anhydrase is shown in FIG. 31C,D and FIG. 31E,F, respectively. Both proteins were eluted from same fraction in the protein separation module (40-45 min for gel based-separation), yet carbonic anhydrase eluted later than myoglobin following elution of the proteins from the matrix processing module. Thus, size-based separation of these proteins was carried out during passage of the fractions in the matrix processing module.

### REFERENCES

All patents, patent applications, patent application publications, and other publications that are cited herein are hereby incorporated by reference as if set forth in their entirety.

It should be understood that the methods, procedures, operations, composition, and systems illustrated in figures may be modified without departing from the spirit of the present disclosure. For example, these methods, procedures, operations, devices and systems may comprise more or fewer steps or components than appear herein, and these steps or components may be combined with one another, in part or in whole.

Furthermore, the present disclosure is not to be limited in terms of the particular embodiments described in this application, which are intended as illustrations of various embodiments. Many modifications and variations can be made without departing from its scope and spirit. Functionally equivalent methods and apparatuses within the scope of the disclosure, in addition to those enumerated herein, will be apparent to those skilled in the art from the foregoing descriptions.

### TERMINOLOGY AND DEFINITIONS

The terminology used herein is for the purpose of describing particular embodiments only, and is not intended to be limiting. With respect to the use of substantially, any plural and/or singular terms herein, those having skill in the art can translate from the plural as is appropriate to the context and/or application. The various singular/plural permutations may be expressly set forth herein for the sake of clarity.

Terms used herein are intended as “open” terms (e.g., the term “including” should be interpreted as “including but not limited to,” the term “having” should be interpreted as “having at least,” the term “includes” should be interpreted as “includes but is not limited to,” etc.).

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into subranges as discussed above.

A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

For clarity, as used herein, the symbols “um” and “μm” have the same meaning as “micron” for a unit of length. Likewise, as used herein, the symbols “ug” and “μg” have the same meaning as “microgram” for a unit of weight. Similarly, as used herein, the symbols “uL” and “μL” have the same meaning as “microliter” for a unit of volume.

The phrase “fluid communication” refers to the ability to transfer a sample in a fluid medium from system, subsystem or module to another system, subsystem or module without having to isolate the sample from the fluid medium. As used herein, “fluid communication” refers to discontinuous transfer of a sample from a fluid medium between one or more systems, subsystems or modules. Examples of discontinuous transfer include on-line hyphenation, isolated retention loops and volumetric holding cells.

The invention claimed is:

1. A system for analyzing a specimen containing the proteome by mass spectrometry, comprising:
a protein separation module;
a matrix processing module; and
a mass spectrometer module,
wherein the protein separation module, the matrix processing module and the mass spectrometer are in fluid communication with one another.

2. The system of claim 1, wherein the protein separation module comprises:
a continuous elution SDS-containing gel for separating proteins of the specimen;
a negatively charged gel trap; and
a collection chamber,
wherein the continuous elution SDS-containing gel and the negatively charged gel trap are in fluid communication with one another and are in electrical communication with an electrophoretic force field such that proteins within the continuous elution SDS-containing gel migrate from the continuous elution SDS-containing gel into the collection chamber.

3. The system of claim 2, wherein the SDS-containing gel comprises a SDS-PAGE gel column having a inner diameter of about 1 mm.

4. The system of claim 2, wherein the negatively charged gel trap comprises a negatively charged acrylamide.

5. The system of claim 2, further comprising a tee fitted with a plurality of valves in fluid communication with a pump and the matrix processing module.

6. The system of claim 1, wherein the matrix processing module is selected from an ion exchange resin and an AF4 device.

7. The system of claim 1, wherein the matrix processing module comprises an ion exchange resin comprising a weak anion exchange resin.

8. The system of claim 1, wherein the matrix processing module comprises an AF4 device having a miniaturized channel.

9. The system of claim 1, wherein the matrix processing module comprises an AF4 device having a channel with a length in the range from about 3.0 cm to about 10.0 cm, a width in the range from about 0.20 cm to about 1.00 cm and a height (thickness) in the range from about 10 microns to about 300 microns.

10. The system of claim 1, wherein the matrix processing module comprises an AF4 device having a channel with a volume in the range from about 3×10⁻⁶ cm³ to about 1.5×10⁻³ cm³, and a surface area in the range from about 0.30 cm² to about 5.00 cm².

11. The system of claim 1, wherein the mass spectrometer module is selected from SLD-MS, MALDI-MS, ESI-MS and MS/MS.

12. A subsystem for preparing a specimen containing the proteome for analysis by mass spectrometry, comprising:
a protein separation module; and
a matrix processing module,
wherein the protein separation module and the matrix processing module are in fluid communication with one another.

13. The subsystem of claim 12, wherein the protein separation module comprises:
a continuous elution SDS-containing gel for separating proteins of the specimen;
a negatively charged gel trap; and
a collection chamber,
wherein the continuous elution SDS-containing gel and the negatively charged gel trap are in fluid communication with one another and are in electrical communication with an electrophoretic force field such that proteins within the continuous elution SDS-containing gel migrate from the continuous elution SDS-containing gel into the collection chamber.

14. The subsystem of claim 13, wherein the SDS-containing gel comprises a SDS-PAGE gel column having a inner diameter of about 1 mm.

15. The subsystem of claim 13, wherein the negatively charged gel trap comprises a negatively charged acrylamide.

16. The subsystem of claim 13, further comprising a tee fitted with a plurality of valves in fluid communication with a pump and the matrix processing module.

17. The subsystem of claim 12, wherein the matrix processing module is selected from an ion exchange resin and an AF4 device.

18. The subsystem of claim 12, wherein the matrix processing module comprises an AF4 device having a channel with a length in the range from about 3.0 cm to about 10.0 cm, a width in the range from about 0.20 cm to about 1.00 cm and a height (thickness) in the range from about 10 microns to about 300 microns.

19. The subsystem of claim 12, wherein the matrix processing module comprises an AF4 device having a channel with a volume in the range from about 3×10⁻⁶ cm³ to about 1.5×10⁻³ cm³, and a surface area in the range from about 0.30 cm² to about 5.00 cm².

20. A method of analyzing a specimen containing the proteome by mass spectrometry, comprising:
injecting a protein extract obtained from the specimen into a protein separation module to separate the protein extract into protein fractions having discrete molecular masses, wherein the separation is effected using a continuous SDS-PAGE gel column and the protein fractions having discrete molecular masses are eluted in solution form the continuous SDS-PAGE gel column and collected into a collection chamber having a negatively charged acrylamide trap;
flowing the collected protein fractions having discrete molecular masses into a matrix processing module in fluid communication with the protein separation module to produce a matrix-free, protein-containing eluate, wherein the matrix processing module comprises an AF4 device having a miniaturized channel with one of the following properties:
a length in the range from about 3.0 cm to about 10.0 cm, a width in the range from about 0.20 cm to about 1.00 cm and a height (thickness) in the range from about 10 microns to about 300 microns; or
a volume in the range from about 3×10⁻⁶ cm³ to about 1.5×10⁻³ cm³ and a surface area in the range from about 0.30 cm² to about 5.00 cm²;
wherein the matrix processing module is configured to remove matrix components that interfere with mass spectrometry analysis of proteins; and
flowing the matrix-free protein-containing eluate into a mass spectrometer in fluid communication with the matrix processing module to analyze protein.
